(19) United States
(12) Patent Application Publication
(10) Pub. No.: US 2023/0002758 A1
(43) Pub. Date: Jan. 5, 2023

(54) TETHERED RIBOSOMES AND METHODS OF MAKING AND USING THEREOF

(71) Applicant: Northwestern University, Evanston, IL (US)

(72) Inventors: Do Soon Kim, Evanston, IL (US); Michael Christopher Jewett, Evanston, IL (US)

(21) Appl. No.: 17/841,618

(22) Filed: Jun. 15, 2022

Related U.S. Application Data
(60) Provisional application No. 63/202,555, filed on Jun. 16, 2021.

(51) Int. Cl. C12N 15/10; C12P 21/02; C12P 19/34; C12N 15/70

(52) U.S. Cl. CPC C12N 15/1041; C12P 21/02; C12P 19/34; C12N 15/70

(57) ABSTRACT

The present disclosure relates to methods to evolve macromolecular machines and improved macromolecular machines identified and made by the methods. In some embodiments, the improved macromolecular machines include improved tethered ribosomes. Also disclosed are systems and methods for making and using the improved tethered ribosomes.

### CROSS-REFERENCE TO RELATED APPLICATIONS

This application claims the benefit of U.S. Provisional Patent Application No. 63/202,555 filed Jun. 16, 2021, the entire content of which is incorporated herein by reference in its entirety.

### STATEMENT REGARDING FEDERALLY SPONSORED RESEARCH OR DEVELOPMENT

This invention was made with government support under W911NF-16-1-0372 awarded by the Army Research Office, Department of Defense, and 1716766 awarded by the National Science Foundation. The government has certain rights in the invention.

### SEQUENCE LISTING

A Sequence Listing accompanies this application and is submitted as an ASCII text file of the sequence listing named “702581_02153_ST25.txt” which is 116,528 bytes in size and was created on May 19, 2022. The sequence listing is electronically submitted via EFS-Web with the application and is incorporated herein by reference in its entirety.

### FIELD OF INVENTION

The present disclosure relates to methods to evolve macromolecular machines and improved macromolecular machines identified and made by the methods. In some embodiments, the improved macromolecular machines include improved tethered ribosomes. Also disclosed are systems and methods for making and using the same.

### BACKGROUND

The ribosome is a molecular machine responsible for the polymerization of α-amino acids into proteins. In all kingdoms of life, the ribosome is made up of two subunits. In bacteria, these correspond to the small (30S) subunit and the large (50S) subunit. The 30S subunit contains the 16S ribosomal RNA (rRNA) and 21 ribosomal proteins (r-proteins), and is involved in translation initiation and decoding the mRNA message. The 50S subunit contains the 5S and 23S rRNAs and 33r-proteins, and is responsible for accommodation of amino acid substrates, catalysis of peptide bond formation, and protein excretion.

The extraordinarily versatile catalytic capacity of the ribosome has driven extensive efforts to harness it for novel functions, such as reprogramming the genetic code. For example, the ability to modify the ribosome's active site to work with substrates beyond those found in nature such as mirror-image (D-α-) and backbone-extended (β- and γ-) amino acids, could enable the synthesis of new classes of sequence-defined polymers to meet many goals of biotechnology and medicine. Unfortunately, cell viability constraints limit the alterations that can be made to the ribosome.

To bypass this limitation, recent developments have focused on the engineering of specialized ribosome systems. The concept is to create an independent, or orthogonal, translation system within the cell dedicated to production of one or a few target proteins while wild-type ribosomes continue to synthesize genome-encoded proteins to ensure cell viability. Pioneering efforts by Hui and DeBoer, and subsequent improvements by Chin and colleagues, first created a specialized small ribosomal subunit. By modifying the Shine-Dalgarno (SD) sequence of an mRNA and the corresponding anti-Shine Dalgarno (ASD) sequence in 16S rRNA, they generated orthogonal 30S subunits capable of primarily translating a specific kind of engineered mRNA, while largely excluding them from translating endogenous cellular mRNAs. These advances enabled the selection of mutant 30S ribosomal subunits capable of re-programming cellular logic and enabling new decoding properties.

Unfortunately, such techniques have been restricted to the small subunit because the large subunits freely exchange between pools of native and orthogonal 30S. This limited the engineering potential of the large subunit, which contains the peptidyl transferase center (PTC) active site and the nascent peptide exit tunnel. This limitation has been addressed with a fully orthogonal ribosome (termed Ribo-T), whereby the small and large subunits are tethered together via helix h44 of the 16S rRNA and helix H101 of the 23S rRNA.

Since the initial discovery of Ribo-T and a subsequent stapled design¹⁵, new orthogonal Ribo-T/mRNA pairs as well as tether sequences have been optimized using directed evolution methods^{9,14}. Specifically, tether residues have been randomized in sequence but not in length⁹, or mutations to surrounding residues surrounding a fixed RNA linker (the J5/5a junction from the Tetrahymena group I intron) have been investigated¹⁴. Despite the improvement, the potential of tethered ribosome systems remains limited by their low activity.

The untapped potential and existing inefficiencies of tethered ribosome systems motivate the need for new directed evolution-based approaches to engineer these systems for improving their activity.

### SUMMARY

Disclosed herein are tethered ribosomes and methods of making and using the ribosomes. Also disclosed are novel methods for evolving macromolecular machines, termed “Evolink.”

Disclosed herein are engineered ribosomes. In some embodiments, the engineered ribosomes comprise a) a small subunit comprising a 16S rRNA polynucleotide sequence or variant thereof; b) a large subunit comprising a 23S rRNA polynucleotide sequence or variant thereof, c) a linking moiety comprising a T1 polynucleotide domain and a T2 polynucleotide domain, wherein the linking moiety links the 16s RNA and the 23S rRNA, thereby linking the large and small ribosomal subunits. In some embodiments, the linking moiety covalently bonds helix 101 of the 23S rRNA large subunit to helix 44 of the 16s rRNA of the small subunit. In some embodiments, the T1 polynucleotide domain comprises 5′-GUUAUA-3′ or 5′-AGUCAAUAA-3′ and the T2 polynucleotide domain comprises 5′-UCACAAG-3′; or 5′-GACCUUCG-3′. In some embodiments, the T1 polynucleotide domain comprises 5′-GUUAUA-3′ and the T2 polynucleotide domain comprises 5′-UCACAAG-3′. In some embodiments, the T1 polynucleotide domain comprises 5′-AGUCAAUAA-3′ and T2 polynucleotide comprises 5′-GACCUUCG-3′. In some embodiments, the engineered ribosome comprises SEQ ID NO: 1.

Also disclosed are polynucleotides encoding the rRNA of the engineered ribosomes, such as, for example, SEQ ID NO: 1, and cells comprising the polynucleotides. Also disclosed are methods for preparing a sequence-defined polymer using the engineered ribosomes disclosed herein.

Also disclosed are methods for evolving molecular machines comprising RNA and/or protein regions of interest that are far apart in primary sequence, but proximal in three-dimensional space. In some embodiments, the methods comprise a design step, a build step, a test step, and an analyze step, the test step involving Evolink, comprising a first PCR, a ligation, and a second PCR.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A-1C. (A) illustrates the secondary structure of a large subunit rRNA (101) and a small subunit rRNA (102) of a wild-type ribosome. (B) illustrates a tethered ribosome having a large subunit, a small subunit, and a linking moiety (103). (C) provides an illustrative transcript for a tethered ribosome rRNA.

FIG. 2A-2E provides illustrations of the Ribo-T system. (A) schematic of the Ribo-T showing tether and orthogonal ribosome binding site in the 30S subunit. (B) The tether is optimized in cells growing exclusively from the Ribo-T plasmid. (C) Previously published Ribo-T tether sequence, T1 and T2. (D) Orthogonal function evolved for Ribo-T. (E) Previously published orthogonal mRNA (o-mRNA) Shine-Dalgarno (SD) sequence and orthogonal 16S rRNA anti-SD (o-ASD) sequence shown.

FIG. 3A-3C. (A) Ribo-T v1 previously published tether sequences T1 and T2. (B) Ribo-T v2 previously published tether sequences T1 and T2. (C) T1 and T2 regions evaluated for optimization as described herein.

FIG. 4A-4C. Overview of Evolink and tethered ribosome design and evolution. (A) RNA- and protein based enzymes with regions that are distal in primary sequence but proximate in 3D space (Regions 1 & 2, blue and red, respectively), and likely functionally linked. Molecular biology steps of Evolink (PCR-1, LIG-1, PCR-2) to link regions together in a single amplicon that enable overlapping next-generation sequencing (NGS) readouts. DNA oligos (green), can be flexibly designed depending on the machine architecture encoded on a plasmid. (B) Rosetta-predicted structure of a previously reported tethered ribosome showing tethers, denoted T1 and T2, in 3D space as well as likely secondary structure representation. Representative encoding plasmid (right) is shown. (C) The Design, Build, Test, & Analyze evolution scheme. (Test) includes selection, Evolink, and the resulting NGS reads. (Analyze) involves Rosetta modeling to infer tether structure and predicted stability. Results from each round feed into (Design) and (Build).

FIG. 5A-5C. Results of the Broad Sampling Library. (A) Residues targeted in this library (red) depicted with surrounding residues (black) in native secondary structure. (B) Fold-enrichment (log 2) of tether sequence pairs during selection in liquid culture over four time points (1 time point per day for 4 days). (C) Analysis of the NGS results reveals convergence towards 9 and 12 nucleotides for T1 and T2 regions, respectively. Data representative of three independent experiments.

FIG. 6A-6C. Investigation of the Tether-H101 junction. (A) Rosetta modeling of the Ribo-T v2 tether and surrounding residues. The junction (cyan) consists of nucleotides that connect the tether (red) to the rest of H101 (blue) in the 23S rRNA. (B) Secondary structure depiction of the library for testing deletion effects in the junction. (C) Results from Evolink showing convergence towards specific Ribo-T v2 Tether-H101 junction sequence. Heatmap data representative of three independent experiments.

FIG. 7A-7H. Integration and validation of designed junction into library design. (A) The sequence of T1 and T2 tethers selected from the Broad Sampling Library. (B-C) Rosetta modeling of the Tether-23S junction (purple) show significant differences between enforcing or not enforcing (constrained vs. unconstrained, respectively) native base pairing. (D) Rosetta score vs. Root-Mean-Standard-Deviation (RMSD) for constrained and unconstrained models of the enriched sequence. (E) Library with the designed Tether-23S junction, reinforced by three synthetic base pairs (gold). (F) Representative fold-enrichment (log 2) of tether sequences from selection and Evolink on the designed Tether-23S junction library. Data representative of three independent experiments. (G) Heatmap of relative abundance of tether lengths showing convergence towards 6 and 8 nucleotides for T1 and T2, respectively. (H) Rosetta score vs. RMSD for constrained and unconstrained models of an enriched sequence from the designed library.

FIG. 8A-8G. Clonal isolation and test of Ribo-Tv3 function. (A) The final library which combines the designed Tether-23S junction and lengths informed by Evolink results from the Designed Tether-23S junction library. (B) Cartoon schematic for orthogonal sfGFP synthesis. (C) Orthogonal sfGFP synthesis by 16 candidates Ribo-Tv3 tether pairs based on the four most popular T1 and T2 genotypes. Data are from three biological replicates (n=3) and error bars indicate standard deviation, representative of two independent experiments. (D-E) Growth of SQ171 cells living on Ribo-Tv3 and Ribo-Tv2 on rich Luria broth media (D) and minimal M9 media (E). Data are from twenty biological replicates (n=20) and error shown represents a 95% confidence interval on each estimated parameter in the sigmoid curve fit. Data representative of three independent experiments. (F-G) Incorporation of 2-amino-3-(7-(diethylamino)-2-oxo-2H-chromene-3-carboxamido) propanoate (DECP) into a sequence-defined peptide by a purified sample of Ribo-Tv3 (F) and Ribo-T v2 (G) in an in vitro protein synthesis reaction using flexizymes. MALDI data representative of three independent experiments.

FIG. 9 Showing the preparation of DECP-CME (Appendix II).

FIG. 10A-10D Tertiary interactions (RNA:RNA) in the ribosome between regions far apart in primary sequence. (A) Helix 96 (red; nucleotides 2702-2704) of the 23S rRNA base pairs with Helix 57 (green; nucleotides 1455-1457). (B) Helix 88 (orange; nucleotides 2407-2411) of Domain V/Central Protuberance makes contacts with Helix 22 (cyan; nucleotides 412-416) in Domain I. (C) RNA:RNA contacts also exist between the large and small subunits, as evidenced by Helix 8 (blue; nucleotides 147-148 and 174-175) in the 16S rRNA and Helix 56 (green; nucleotides 1446-1447) in the 23S rRNA. (D) Helix 44 (green; nucleotides 1418 & 1483) of the 16S makes possible tertiary contacts with Helix 71 (gold; nucleotides 1947-1948 & 1958-1959).

FIG. 11A-11B. Proof of concept study for library preparation workflow of Evolink. (A) A clonal sample of the tethered ribosome (Ribo-T v2) is linearized using different oligos compatible with multiple ligation protocols. (B) From the different ligation products, generation of final amplicon for next-generation sequencing can happen with a wide range of ligation methods and starting template amounts in the PCR. Gel data representative of two independent experiments.

FIG. 12A-12C. Enrichment of individual genotypes throughout full Evolink experiment. Positively enriched genotypes (purple) and negative enriched genotypes (dark gray) can be tracked throughout multiple time points throughout selection. Genotypes that drop out during selection can also be identified (light gray). Generally, across the three libraries tested in this work, (A) the Broad Sampling Library, (B) the Designed Junction Library, and (C) the Designed Junction+Length Refined Library, log₂-fold enrichment values between −6 to 6 are observed. Enrichment data representative of three independent experiments

FIG. 13A-13C. Distribution of T2 sequences for most enriched T1 sequences. Distribution of T2 sequences for most popular T1 sequences displayed for the three libraries tested ((A)Broad Sampling Library, (B) Designed Junction Library, (C) Designed Junction+Length Sampling Library). Scatter plot represents unique T2 sequences for a given T1 sequence. Violin plot and scatter plot data representative of three independent experiments.

FIG. 14A-14C. Distribution of T1 sequences for most enriched T2 sequences. Distribution of T1 sequences for most popular T2 sequences displayed for the three libraries tested (Broad Sampling Library, Designed Junction Library, Designed Junction+Length Sampling Library). Scatter plot represents unique T1 sequences for a given T2 sequence. Violin plot and scatter plot data representative of three independent experiments

FIG. 15A-15D. Analysis of enriched genotypes from the Broad Sampling Library. Each panel shows an enriched sequence modeled using RNAcofold. For three of the genotypes, (A), (C), and (D), the same tether base pairs are formed in the constrained and unconstrained minimum free energy (MFE) structures. (B) For one of the genotypes, significant rearrangement is observed between the constrained vs. unconstrained MFE structures.

FIG. 16A-16B. Representative constrained and unconstrained 3D models of Designed Junction Library winner. The winning genotype from FIG. 4H was modeled using Rosetta, and representative outputs are shown. In both the (A) unconstrained and (B) constrained model, the Designed Junction residues are predicted to base pair, reinforcing structural stability to this region.

FIG. 17A-17H. Score vs. Root-Mean-Standard-Deviation analysis of FARFAR2 simulations of enriched tether sequences. (A-D) For the Broad Sampling Library, we observe significant differences between simulations that constrained (blue) or did not constrain (orange) 3D structures of the Tether-H101 junction. Of the four modeled genotypes, two sequence (C-D) exhibit particularly significant differences, hinting at structural instability in the Tether-H101 junction. (E-H) When similar simulations are performed with enriched tether sequences from the Designed Junction Library (designed sequences at the Tether-H101 junction), the results of FARFAR2 simulations reach similarly low scores in constrained vs. unconstrained modeling runs.

FIG. 18. Heatmap of lengths by enrichment for the Designed Junction+Length Refined Library. Lengths of 6 and 8 nt are enriched, as seen in the Designed Junction Library. No constructs of length 9 nt for the T2 region was observed in the final time point. Heatmap data (relative frequency of next-generation sequencing read) representative of three independent experiments.

FIG. 19A-19B. Growth curves and parameters for Ribo-T v3 compared to Ribo-T v2 in cells lacking genomic ribosomal operons. Sigmoidal functions were fit to kinetic data (left) to calculate parameters (right). (A) In rich Luria broth (LB), Ribo-T v2 and Ribo-T v3 have equivalent slopes ˜0.08 A₆₀₀/hour (doubling rates in exponential phase), but Ribo-T v3 has shorter lag time in growth. (B) In minimal M9 media, the difference in slopes and lag are more pronounced. Notably, Ribo-T v2 does not reach full stationary phase in 24 hours while Ribo-T v3 grows to stationary phase between 18-20 hours. Error bars represent one standard deviation calculated for six replicates (n=6). Growth data are representative of three independent experiments, each performed with six replicates.

FIGS. 20A-20B. (A)¹H and (B)¹³C NMR spectra of DECP-CME (5).

FIG. 21. Acylation of microhelix with DECP. The Fx-mediated acylation reaction was monitored using microhelix (a tRNA mimic) under the two different pH (7.5 and 8.8) over 16 h with three different flexizymes (eFx, dFx, and aFx) at 0° C. The highest acylation yield (86%) was found when aFx was used in pH 7.5, which was used to charge the substrate into tRNA^{fMet}(CAU) and incorporate it into the N-terminus of a peptide in vitro. Gel representative of three independent experiments.

FIG. 22A-22B. Characterization of N-terminus functionalized peptide hybridized with DECP. (A) Structure and molecular weight of byproduct peptides in the in vitro translation reaction that are produced. (B) MALDI mass spectrometry data (FIG. 5E) obtained from attempt to incorporate DECP with Ribo-T v3. The truncated peptide (P1) was produced likely because Ribo-T v3 skipped the incorporation of DECP at the initiating codon (AUG) on mRNA. P2 was produced presumably because of the contaminations of either amino acid or Met-charged tRNA (tRNA^{fMet}) when Ribo-T v3 obtained from E. coli cell was supplemented into the in vitro translation reaction. The percent yield (76%) of the target peptide (P3) was determined based on the relative peak area (PA) of P3 over a total amount of the byproducts (P1 and P2) and P3 (i.e., relative yield (%)=Σ of PA (P3)/Σ of PA (P1+P2+P3)×100). MALDI data representative of three independent experiments.

FIG. 23 is a Table showing tether pairs T1 and T2 and the percent improvement in activity relative to RiboT-v2. Tether pairs 1-14 perform better than RiboT-V2, while tether pairs 15 and 16 perform worse than RiboT-v2. The performance of wild-type ribosomes is shown in the last row of the table. Tether pairs were ranked based on metric “Norm_RFU”, which is the normalized GFP yield. The full sequence of modified ribosomal RNA including the 16 tether pairs is provided in the Examples.

FIG. 24A-24B. Cryo-EM structure of the Ribo-Tv3 ribosome at 4.18 angstroms resolution. A 4.18 angstrom density of the Ribo-Tv3 ribosome was generated through single-particle analysis. (A) The 4YBB ribosome structure fit into the Ribo-Tv3 density. (B) Zoom-in on the density of the Ribo-Tv3 tether region with top 10 DRRAFTER models of the tether built into the density.

### DETAILED DESCRIPTION

Terminology

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

A range includes each individual member. Thus, for example, a group having 1-3 members refers to groups having 1, 2, or 3 members.

It should also be understood that, unless clearly indicated to the contrary, in any methods claimed herein that include more than one step or act, the order of the steps or acts of the method is not necessarily limited to the order in which the steps or acts of the method are recited.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

The terms “nucleic acid” and “oligonucleotide,” as used herein, refer to polydeoxyribonucleotides (containing 2-deoxy-DRibose), polyribonucleotides (containing DRibose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)n sequence, where n is in the range from about 20 to about 200). The region of the primer that is sufficiently complementary to the template to hybridize is referred to herein as the hybridizing region.

The term “promoter” refers to a cis-acting DNA sequence that directs RNA polymerase and other trans-acting transcription factors to initiate RNA transcription from the DNA template that includes the cis-acting DNA sequence.

The terms “target, “target sequence”, “target region”, and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, the term “sequence defined polymer” refers to a polymer having a specific primary sequence. A sequence defined polymer can be equivalent to a genetically-encoded defined polymer in cases where a gene encodes the polymer having a specific primary sequence.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, “expression template” refers to a nucleic acid that serves as substrate for transcribing at least one RNA that can be translated into a polypeptide or protein. Expression templates include nucleic acids composed of DNA or RNA. Suitable sources of DNA for use a nucleic acid for an expression template include genomic DNA, plasmid DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “tethered,” “conjoined,” “linked,” “connected,” “coupled” and “covalently-bonded” have the same meaning as modifiers.

As used herein, “tethered ribosome,” “engineered ribosome,” and “Ribo-T” will be used interchangeably.

As used here, “CP” refers to a circularly permuted subunit. As used herein, when CP is followed by “23S” that refers to a circularly permuted 23S rRNA. As used herein, when CP followed by a number may refer to the location of the new 5′ end in a secondary structure, e.g. CP101 means the new 5′ end is in helix 101 of the 23S rRNA, or to the location of the new 5′ nucleotide, e.g. CP2861 means the new 5′ nucleotide is the nucleotide 2861 of the 23 rRNA, depending on context.

As used herein, “translation template” refers to an RNA product of transcription from an expression template that can be used by ribosomes to synthesize polypeptide or protein.

Methods for Improved Molecular Evolution of Biological Machines and Compositions Derived Therefrom

Disclosed herein is a new technique for evolving macromolecular machines, which combines molecular biology techniques with next-generation sequencing to allow co-evolution of functionally-linked residues previous out of reach for next generation sequencing reads with length limitations (˜300 nts). Termed Evolink, this technique is broadly applicable to large RNA or protein machines, and can be implemented with very basic techniques available in many molecular biology laboratories.

Also disclosed herein is a new sequence for an RNA machine, the ribosome, which improves upon the previous tethered ribosome (see e.g., Ref 9). The new ribosome system, termed Ribo-T v3, is capable of orthogonal protein synthesis and improved cellular fitness when supporting life.

Ribo-T v3 features new ribosomal RNA sequences that link together the 16S and 23S rRNA of the small (30S) and large (50S) subunits of the E. coli ribosome. This new RNA sequence was achieved by applying a newly invented technique called Evolink, in which distal regions of a machine (e.g., functional protein or nucleic acid sequence) encoded on a plasmid can be linked together in an amplicon for next-generation reads to enable co-evolution of previously separated parts. Evolink can be applied to any machine encoded on a plasmid, and can link together multiple regions. Such regions are abundant in many macromolecular machines (both protein and RNA), and have been precluded from high throughput evolution due to limitations in assay techniques.

Ribosome engineering is emerging as a powerful approach for expanding the catalytic potential of the protein synthesis apparatus and for elucidating its origin, evolution and function. Because the properties of the engineered ribosome might be detrimental for the general protein synthesis, the designer ribosome needs to be functionally isolated from the translation machinery synthesizing cellular proteins. The initial solution to this problem has been offered by Ribo-T, an engineered ribosome with the tethered subunits which, while translating a desired protein, could be excluded from translation of the cellular proteome. In the present disclosure, the inventors present a new paradigm for designing and evolving macromolecular machines. The inventors herein demonstrate the combination of computational modeling with a molecular biology workflow that enables high-throughput evolution of distant regions in a large molecular machine. To showcase the utility of the approach, the inventors evolved a tethered ribosome which improves upon the previous state-of-the-art by over 50% in orthogonal protein translation.

Applications and Advantages of Evolink

The improved molecular evolution methods for biological machines, and compositions derived therefrom, e.g., improved tethered ribosomes, have many applications and advantages. The following are examples only, and are not intended to be limiting.

Ribosome evolution/engineering (for example towards more efficient non-canonical amino acid incorporation); expanded genetic codes for non-canonical amino acid incorporation; enabling detailed in vivo studies of antibiotic resistance mechanisms, enabling antibiotic development process; biopharmaceutical production; orthogonal circuits in cells; synthetic biology; production of engineered peptides by incorporating new functionality inaccessible to peptides synthesized by native (or wild type) ribosome or their post-translationally modified derivatives; production of novel protease-resistant peptides that could transform medicinal chemistry.

For evolution of the ribosomes, the inventors present a new paradigm for directed evolution that integrates computational structural modeling of RNA machines as well as a new molecular biology technique that enables evolution of distant regions on molecular machines compatible with next-generation sequencing.

This improved upon a previous state-of-the-art design for a tethered ribosome (Ribo-T v2.0, see Ref. 9). It outperforms Ribo-T v2.0 in both supporting cellular life (faster and more robust growth) as well as orthogonal protein production (improved orthogonal GFP synthesis).

Improvements to orthogonal ribosomes could play a vital role in successful directed evolution towards new functions, such as new polymerization chemistries and orthogonal genetic circuits.

The inventors further show compatibility of orthogonal, tethered ribosomes with other synthetic translation machinery, specifically the flexizyme system for non-standard amino acid incorporation to produce a peptide containing a coumarin derivative non-canonical monomer in an in vitro translation reaction. This combination of engineered translation machinery has not previously been shown.

The novel evolutionary molecular method disclosed herein greatly increases throughput of directed evolution efforts on large protein or RNA enzymes. The unmet need is the current limitation in the number of genotypes that can be linked to advantageous phenotypes. Notably, it is impossible to evolve sequence-distal parts of molecular machines and interactions between those sequences although based on structure they are likely linked in function. The invention described herein allows a research group to rapidly assess which parts of macromolecular machines are functionally linked, and then to perform directed evolution on them with readouts that allow them to link sequence-distal parts in Next Generation Sequencing (NGS) readouts without having to rely on clonal screening or using statistics to infer functional linkage. This invention could increase throughput by orders of magnitude and with greater fidelity than previously available methods.

Engineered Ribosomes

Engineered ribosomes and methods of making and using the ribosomes, are described in U.S. Pat. No. 10,590,456, Ref. 9, and Ref. 18, each of which is incorporated herein by reference in its entirety.

The engineered ribosome comprises a small subunit, a large subunit, and a linking moiety, wherein the linking moiety tethers the small subunit with the large subunit. In some embodiments, the engineered ribosome is capable of supporting translation of a sequence-defined polymer. In some embodiments, the engineered ribosome comprises a linking moiety that links the 16S and 23S rRNA of the small (30S) and large (50S) subunits of the E. coli ribosome.

In the following discussion, the rRNA component of ribosomes is the focus. As is well known in the art, ribosomes, including the engineered ribosomes disclosed herein, comprise ribosomal proteins as well as RNA. For example, bacterial ribosomes, such as E. coli ribosomes, include 31 ribosomal proteins in the 50S (large) subunit, and 21 ribosomal proteins in the 30S (small) subunit. Ribosomal proteins and methods of making ribosomes are well known in the art (see e.g., references above). While the RNA is the focus of the discussion, it is to be understood that ribosomes and their subunits also include ribosomal proteins.

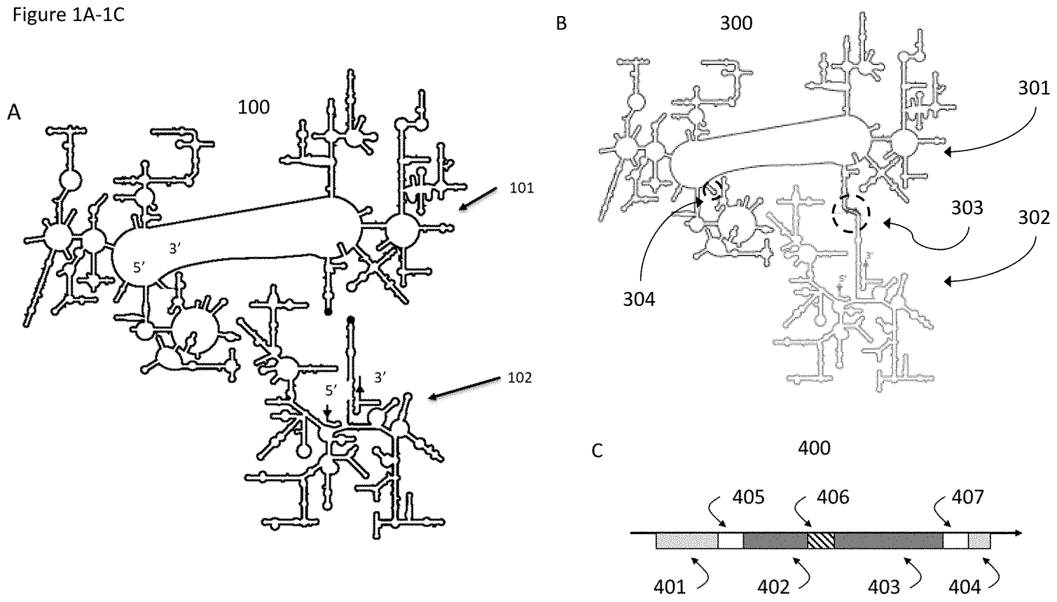

In contrast to a naturally occurring ribosome, the engineered ribosome has a large and a small subunit that are not separable. FIG. 1A depicts a portion of a wild-type ribosome 100 having a small subunit and a large subunit that are separable. FIG. 1A illustrates the secondary structure of a large subunit rRNA 101 and a small subunit rRNA 102 that together form a portion of a functional ribosome.

An embodiment of a portion of an engineered tethered ribosome is illustrated in FIG. 1B, which illustrates the secondary structure of an exemplary large subunit rRNA 301 and an exemplary small subunit rRNA 302 that together form a portion of a functional engineered ribosome. The engineered ribosome comprises a large subunit rRNA 301, a small subunit rRNA 302, and a linking moiety 303 that tethers the small subunit with the large subunit. The engineered ribosome may also comprise a connector 304, that closes the ends of a native large subunit rRNA.

Large Subunit

The large ribosome subunit 301 comprises a subunit capable of joining amino acids to form a polypeptide chain. The large subunit 301 may comprise a ribosomal RNA comprising a first large subunit domain (“L1 polynucleotide domain” or “L1 domain”), a second large subunit domain (“L2 polynucleotide domain” or “L2 domain”), and a connector domain (“C polynucleotide domain” or “C domain”) 304, wherein the L1 domain is followed, in order, by the C domain and the L2 domain, from 5′ to 3′.

FIG. 1C illustrates an example of an rRNA gene 400 that encodes the engineered ribosome rRNA 300, and provides an alternative representation for understanding the engineered ribosome. The encoding polynucleotide 400 may comprise different sequences that encode the various domains of the engineered ribosome rRNA 300. As illustrated in FIG. 1C, the polynucleotide encoding the large subunit rRNA 301 comprises the polynucleotide encoding the L1 domain 402, the polynucleotide encoding the C domain 406, and the polynucleotide encoding the L2 domain 403.

In some embodiment, the large subunit rRNA 301 may be a permuted variant of a separable large subunit rRNA. In some embodiments, the permuted variant is a circularly permuted variant of a separable large subunit rRNA. The separable large subunit may be any functional large subunit. In some embodiments, the separable large subunit may be a 23S rRNA. In some embodiments, the separable large subunit comprises a wild-type large subunit rRNA. In some embodiments, the separable large subunit is a wild-type 23S rRNA. In some embodiments, the separable large subunit is at least 90% identical, at least 91% identical, at least 92% identical, at least 93% identical, at least 94% identical, at least 95% identical, at least 96% identical, at least 97% identical, at least 98% identical, or at least 99% identical to a wild-type 23S rRNA.

In some embodiment, if the large subunit 301 is a permuted variant of a large subunit rRNA, then the polynucleotide sequences consisting essentially of the L2 domain, followed by the L1 domain, from 5′ to 3′, may be substantially identical to a large subunit rRNA. In some embodiments, the polynucleotide sequence consisting essentially of the L2 domain followed by sequence consisting essentially of the L1 domain, from 5′ to 3′, is at least 90% identical, at least 91% identical, at least 92% identical, at least 93% identical, at least 94% identical, at least 95% identical, at least 96% identical, at least 97% identical, at least 98% identical, or at least 99% identical to the large subunit rRNA.

In some embodiments where the large subunit 301 is a permuted variant of a separable large subunit rRNA, the large subunit 301 may further comprise a C domain 304 that connects the native 5′ and 3′ ends of the separable large subunit rRNA. The C domain may comprise a polynucleotide having a length ranging from 1-200 nucleotides. In some embodiments, the C domain 304 comprises a polynucleotide having a length ranging from 1-150 nucleotides 1-100 nucleotides, 1-90 nucleotides, from 1-80 nucleotides, 1-70 nucleotides, 1-60 nucleotides, 1-50 nucleotides, 1-40 nucleotides, 1-30 nucleotides, 1-20 nucleotides, 1-10 nucleotides, 1-9 nucleotides, 1-8 nucleotides, 1-7 nucleotides, 1-6 nucleotides, 1-5 nucleotides, 1-4 nucleotides, 1-3 nucleotides, or 1-2 nucleotides. In certain embodiments, the C domain comprises a GAGA polynucleotide.

Small Subunit

The small subunit 302 is capable of binding mRNA. The small subunit 302 comprises a first small subunit rRNA domain (“S1 polynucleotide domain” or “S1 domain”) and a second small subunit domain (“S2 polynucleotide domain” or “S2 domain”), wherein the S1 domain is followed, in order, by S2 domain, from 5′ to 3′. Referring again to FIG. 1C, the polynucleotide encoding the small subunit rRNA 302 comprises the polynucleotide encoding the S1 domain 401 and the polynucleotide encoding the S2 domain 404.

The small subunit rRNA 302 may be a permuted variant of a separable small subunit rRNA. In certain embodiments, the permuted variant is a circularly permuted variant of a separable small subunit rRNA. The separable small subunit may be any functional small subunit. In certain embodiments, the separable small subunit may be a 16S rRNA. In certain embodiments, the separable small subunit is a wild-type small subunit rRNA. In specific embodiments, the separable small subunit is a wild-type 23S rRNA. In some embodiments, the separable small subunit is at least 90% identical, at least 91% identical, at least 92% identical, at least 93% identical, at least 94% identical, at least 95% identical, at least 96% identical, at least 97% identical, at least 98% identical, or at least 99% identical to the small subunit rRNA.

In some embodiments, if the small subunit 302 is a permuted variant of a small subunit rRNA, then the polynucleotide sequence consisting essentially of the S1 domain followed by the polynucleotide sequence consisting essentially of the S2 domain, from 5′ to 3′, may be substantially identical to a small subunit rRNA. In certain embodiments, the polynucleotide sequence consisting essentially of the S1 domain followed by the S2 domain, from 5′ to 3′, is at least 90% identical, at least 91% identical, at least 92% identical, at least 93% identical, at least 94% identical, at least 95% identical, at least 96% identical, at least 97% identical, at least 98% identical, or at least 99% identical to the small subunit rRNA.

The small subunit may further comprise a modified-anti-Shine-Dalgarno sequence. In some embodiments, the modified anti-Shine-Dalgarno sequence facilitates the translation of templates having a complementary Shine-Dalgarno sequence different from an endogenous cellular mRNA.

Linking Moiety

Referring again to FIG. 1B, the linking moiety 303 tethers the small subunit 302 with the large subunit 301. In certain embodiments the linking moiety covalently bonds a helix of the large subunit 301 to a helix of the small subunit 302.

In some embodiments, the linking moiety comprises a first tether domain (“T1 polynucleotide domain” or “T1 domain”) and a second tether domain (“T2 polynucleotide domain” or “T2 domain”). Referring again to FIGS. 1B and 1C the polynucleotide encoding the linking moiety 303 comprises the polynucleotide encoding the T1 domain 405 and the polynucleotide encoding the T2 domain 407.

In some embodiments, the T1 domain links the S1 domain and the L1 domain, wherein the S1 domain is followed, in order, by the T1 domain and the L1 domain, from 5′ to 3′. In some embodiments, the T1 domain comprises a polynucleotide having a length ranging from 5-200 nucleotide, 5-150 nucleotides, 5-100 nucleotides, 5-90 nucleotide, 5-80 nucleotides, 5-70 nucleotides, 5-60 nucleotides, 5-50 nucleotides, 5-40 nucleotides, 5-30 nucleotides, or 5-20 nucleotides, including polynucleotides having 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In some embodiments, T1 comprises polyadenine. In some embodiments, T1 comprises polyuridine. In some embodiments, T1 comprises an unstructured polynucleotide. In some embodiments, T1 comprises nucleotides that base-pairs with the T2 domain.

In some embodiments, the T2 domain links that L2 domain and the S2 domain, wherein the L2 domain is followed, in order, by the T2 domain and the S2 domain, from 5′ to 3′. In some embodiments, the T2 domain comprises a polynucleotide having a length ranging from 5-200 nucleotides, 5-150 nucleotides, 5-100 nucleotides, 5-90 nucleotide, 5-80 nucleotides, 5-70 nucleotides, 5-60 nucleotides, 5-50 nucleotides, 5-40 nucleotides, 5-30 nucleotides, or 5-20 nucleotides, including polynucleotides having 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In certain embodiments, T1 comprises polyadenine. In certain embodiments, T2 comprises polyuridine. In certain embodiments, T12comprises an unstructured polynucleotide. In certain embodiments, T2 comprises nucleotides that base-pairs with the T1 domain.

In embodiments having a T1 domain and a T2 domain, the T1 domain and the T2 domain may have the same number of polynucleotides. In other embodiments, the T1 domain and the T2 domain may have a different number of polynucleotides.

In some embodiments, the engineered ribosome may comprise a S1 domain followed, in order, by a T1 domain, a L1 domain, a C domain, a L2 domain, a T2 domain, and a S2 domain, from 5′ to 3′. In specific embodiments, the engineered ribosome may consist essentially of a S1 domain followed, in order, by a T1 domain, a L1 domain, a C domain, a L2.

In some embodiments, the ribosomal RNA and the linking moiety of an engineered ribosome comprises the general structure shown below, from 5′ to 3′, wherein 16S (5′) represents S1, 23S includes L1 and L2, and optionally, a connector (not shown), and 16S(3′) represents S2:

In some embodiments, the T1 domain comprises 5′-GUUAUA-3′ and the T2 domain comprises 5′-UCACAAG-3′. In some embodiments, the T1 domain comprises 5′-AGUCAAUAA-3′ and T2 comprises 5′-GACCUUCG-3′.

An engineered ribosome, which includes T1 5′-AGUCAAUAA-3′ and T2 5′-GACCUUCG-3′ and which comprises a variant of a 16S and a 23S rRNA sequence, adapted to accommodate the T1 and T2 sequences as disclosed herein, is termed Ribo-T v3 and is shown below as SEQ ID NO: 1.

5′
aauugaagaguuugaucauggcucagauugaacgcuggcggcaggccuaacacaugcaagucgaacggua
acaggaagaagcuugcuucuuugcugacgaguggcggacgggugaguaaugucugggaaacugccugaug
gagggggauaacuacuggaaacgguagcuaauaccgcauaacgucgcaagaccaaagagggggaccuucg
ggccucuugccaucggaugugcccagaugggauuagcuaguaggugggguaacggcucaccuaggcgacg
aucccuagcuggucugagaggaugaccagccacacuggaacugagacacgguccagacuccuacgggagg
cagcaguggggaauauugcacaaugggcgcaagccugaugcagccaugccgcguguaugaagaaggccuu
cggguuguaaaguacuuucagcggggaggaagggaguaaaguuaauaccuuugcucauugacguuacccg
cagaagaagcaccggcuaacuccgugccagcagccgcgguaauacggagggugcaagcguuaaucggaau
uacugggcguaaagcgcacgcaggcgguuuguuaagucagaugugaaauccccgggcucaaccugggaac
ugcaucugauacuggcaagcuugagucucguagagggggguagaauuccagguguagcggugaaaugcgu
agagaucuggaggaauaccgguggcgaaggcggcccccuggacgaagacugacgcucaggugcgaaagcg
uggggagcaaacaggauuagauacccugguaguccacgccguaaacgaugucgacuuggagguugugccc
uugaggcguggcuuccggagcuaacgcguuaagucgaccgccuggggaguacggccgcaagguuaaaacu
caaaugaauugacgggggcccgcacaagcgguggagcaugugguuuaauucgaugcaacgcgaagaaccu
uaccuggucuugacauccacggaaguuuucagagaugagaaugugccuucgggaaccgugagacaggugc
ugcauggcugucgucagcucguguugugaaauguuggguuaagucccgcaacgagcgcaacccuuauccu
uuguugccagcgguccggccgggaacucaaaggagacugccagugauaaacuggaggaagguggggauga
cgucaagucaucauggcccuuacgaccagggcuacacacgugcuacaauggcgcauacaaagagaagcga
ccucgcgagagcaagcggaccucauaaagugcgucguaguccggauuggagucugcaacucgacuccaug
aagucggaaucgcuaguaaucguggaucagaaugccacggugaauacguucccgggccuuguacacaccg
cccgucacaccaugggaguggguugcaaaagaaguagguagcuuaaccagucaauaagucuugagcuaac
cgguacuaaugaaccgugaggcuuaaccgagagguuaagcgacuaagcguacacgguggaugcccuggca
gucagaggcgaugaaggacgugcuaaucugcgauaagcgucgguaaggugauaugaaccguuauaaccgg
cgauuuccgaauggggaaacccaguguguuucgacacacuaucauuaacugaauccauagguuaaugagg
cgaaccgggggaacugaaacaucuaaguaccccgaggaaaagaaaucaaccgagauucccccaguagcgg
cgagcgaacggggagcagcccagagccugaaucaguguguguguuaguggaagcgucuggaaaggcgcgc
gauacagggugacagccccguacacaaaaaugcacaugcugugagcucgaugaguagggcgggacacgug
guauccugucugaauauggggggaccauccuccaaggcuaaauacuccugacugaccgauagugaaccag
uaccgugagggaaaggcgaaaagaaccccggcgaggggagugaaaaagaaccugaaaccguguacguaca
agcagugggagcacgcuuaggcgugugacugcguaccuuuuguauaaugggucagcgacuuauauucugu
agcaagguuaaccgaauaggggagccgaagggaaaccgagucuuaacugggcguuaaguugcaggguaua
gacccgaaacccggugaucuagccaugggcagguugaagguuggguaacacuaacuggaggaccgaaccg
acuaauguugaaaaauuagcggaugacuuguggcugggggugaaaggccaaucaaaccgggagauagcug
guucuccccgaaagcuauuuagguagcgccucgugaauucaucuccggggguagagcacuguuucggcaa
gggggucaucccgacuuaccaacccgaugcaaacugcgaauaccggagaauguuaucacgggagacacac
ggcgggugcuaacguccgucgugaagagggaaacaacccagaccgccagcuaaggucccaaagucauggu
uaagugggaaacgaugugggaaggcccagacagccaggauguuggcuuagaagcagccaucauuuaaaga
aagcguaauagcucacuggucgagucggccugcgcggaagauguaacggggcuaaaccaugcaccgaagc
ugcggcagcgacgcuuaugcguuguuggguaggggagcguucuguaagccugcgaaggugugcugugagg
caugcuggagguaucagaagugcgaaugcugacauaaguaacgauaaagcgggugaaaagcccgcucgcc
ggaagaccaaggguuccuguccaacguuaaucggggcagggugagucgaccccuaaggcgaggccgaaag
gcguagucgaugggaaacagguuaauauuccuguacuugguguuacugcgaaggggggacggagaaggcu
auguuggccgggcgacgguugucccgguuuaagcguguaggcugguuuuccaggcaaauccggaaaauca
aggcugaggcgugaugacgaggcacuacggugcugaagcaacaaaugcccugcuuccaggaaaagccucu
aagcaucagguaacaucaaaucguaccccaaaccgacacagguggucagguagagaauaccaaggcgcuu
gagagaacucgggugaaggaacuaggcaaaauggugccguaacuucgggagaaggcacgcugauauguag
gugaggucccucgcggauggagcugaaaucagucgaagauaccagcuggcugcaacuguuuauuaaaaac
acagcacugugcaaacacgaaaguggacguauacggugugacgccugcccggugccggaagguuaauuga
ugggguuagcgcaagcgaagcucuugaucgaagccccgguaaacggcggccguaacuauaacgguccuaa
gguagcgaaauuccuugucggguaaguuccgaccugcacgaauggcguaaugauggccaggcugucucca
cccgagacucagugaaauugaacucgcugugaagaugcaguguacccgcggcaagacggaaagaccccgu
gaaccuuuacuauagcuugacacugaacauugagccuugauguguaggauaggugggaggcuuugaagug
uggacgccagucugcauggagccgaccuugaaauaccacccuuuaauguuugauguucuaacguugaccc
guaauccggguugcggacagugucugguggguaguuugacuggggcggucuccuccuaaagaguaacgga
ggagcacgaagguuggcuaauccuggucggacaucaggagguuagugcaauggcauaagccagcuugacu
gcgagcgugacggcgcgagcaggugcgaaagcaggucauagugauccggugguucugaauggaagggcca
ucgcucaacggauaaaagguacuccggggauaacaggcugauaccgcccaagaguucauaucgacggcgg
uguuuggcaccucgaugucggcucaucacauccuggggcugaaguaggucccaaggguauggcuguucgc
cauuuaaagugguacgcgagcuggguuuagaacgucgugagacaguucggucccuaucugccgugggcgc
uggagaacugaggggggcugcuccuaguacgagaggaccggaguggacgcaucacugguguucggguugu
caugccaauggcacugcccgguagcuaaaugcggaagagauaagugcugaaagcaucuaagcacgaaacu
ugccccgagaugaguucucccugacccuuuaaggguccugaaggaacguugaagacgacgacguugauag
gccggguguguaaggacgaccuucgggagggcgcuuaccacuuugugauucaugacuggggugaagucgu
aacaagguaaccguaggggaaccugcgguuggaucaccuccuua 3′.

Mutations

In certain embodiments, the engineered ribosomes disclosed herein, such as Ribo-T v3, may comprise one or more mutations (in addition to those of the rRNA of Ribo-T V3, for example). In some embodiments the mutation is a change-of-function mutation. A change-of-function mutation may be a gain-of-function mutation or a loss-of-function mutation. A gain-of-function mutation may be any mutation that confers a new function. A loss-of-function mutation may be any mutation that results in the loss of a function possessed by the parent.

In some embodiments, the change-of-function mutation may be in the peptidyl transferase center of the ribosome. In specific embodiments, the change-of-function mutation may be in an A-site of the peptidyl transferase center. In other embodiments, the change-of-function mutation may be in the exit tunnel of the engineered ribosome.

In some embodiments the change-of-function mutation may be an antibiotic resistance mutation. The antibiotic resistance mutation may be either in the large subunit or the small subunit. In some embodiments antibiotic resistance mutation may render the engineered ribosome resistant to an aminoglycoside, a tetracycline, a pactamycin, a streptomycin, an edein, or any other antibiotic that targets the small ribosomal subunit. In some embodiments antibiotic resistance mutation may render the engineered ribosome resistant to a macrolide, a chloramphenicol, a lincosamide, an oxazolidinone, a pleuromutilin, a streptogramin, or any other antibiotic that targets the large ribosomal subunit.

Methods

In some embodiments, methods for preparing a sequence defined polymer are provided. In some embodiments, an engineered ribosome as disclosed herein (e.g., RiboT-v3, or functional variants thereof), is contacted with a nucleic acid encoding the sequence defined polymer under conditions for transcription (if the nucleic acid encoding the sequence defined polymer comprises DNA) by transcriptional components, and/or translation (if the nucleic acid encoding the sequence defined polymer comprises mRNA) by the tethered ribosomes. In some embodiments, translation by the tethered ribosomes may include the use non-canonical or unnatural codons and corresponding tRNAs (e.g., using the flexizyme system). Such codons, in combination with a system such as flexizyme, may allow for the production of polymers comprising, for example, non-canonical amino acids, or non-amino acid monomers.

In some embodiments, conditions for translation by the tethered ribosomes may include the use of tethered ribosomes comprising modified anti Shine-Dalgarno sequences, and mRNA comprising complementary modified Shine-Dalgarno sequences.

In some embodiments, the sequence defined polymer is prepared in vitro, for example, in a ribosome-depleted cellular extract or purified translation system.

In some embodiments, the sequence defined polymer is prepared in vivo, for example, in a host cell, such as a bacterial host cell, e.g., an Escherichia coli cell.

Polynucleotides

Disclosed herein are polynucleotides encoding the rRNA of the engineered ribosomes of the present technology (e.g., RiboT-v3, or functional variants thereof). In some embodiments, the polynucleotide comprise a vector. In some embodiments, a vector encoding the rRNA of an engineered ribosome of the present technology also encodes a gene, gene fragment, or other nucleic acid sequence that after transcription, can be translated by the engineered ribosomes. By way of example, in some embodiments, the gene, gene fragment, or other nucleic acid sequence is first transcribed, either in vitro or in vivo (e.g., by bacterial host cell transcription machinery) and is then translated by the engineered ribosomes. In some embodiments, a gene, gene fragment, or other nucleic acid sequence is provided as a separate vector or as a separate nucleic acid (either as DNA or mRNA).

Cells

Disclosed herein are cells comprising one or more polynucleotides encoding rRNA of the engineered ribosomes of the present technology (e.g., RiboT-v3, or functional variants thereof). In some embodiments, one or more of the polynucleotides comprises a vector. In some embodiments, the cells express the encoded rRNA and comprise a functional tethered ribosome as described herein (e.g., RiboT-v3, or functional variants thereof). In some embodiments, the cell comprises a mammalian cell, a yeast cell, an insect cell, an algal cell, a plant cell, a protozoan cell, or a bacterial cell. In some embodiments, the cells is an Escherichia coli cell.

In some embodiments, the cell comprises a first protein translation mechanism and a second protein translation mechanism. In some embodiments, the first protein translation mechanism comprises a ribosome, wherein the ribosome lacks a linking moiety between the large subunit and the small subunit. In some embodiments, the first translation mechanism comprises canonical ribosomes. In some embodiments, the first translation mechanism comprises non-canonical ribosomes. In some embodiments, the second protein translation mechanism comprises an engineered, tethered ribosome as disclosed herein (e.g., RiboT-v3, or functional variants thereof).

Methods of Directed Evolution

Disclosed herein are methods for directed evolution of a target nucleic acid sequence. In some embodiments, the target nucleic acid sequence comprises at least two regions of interest, wherein the regions of interest are separated by an intervening sequence of at least 300 nucleotides in length. In some embodiments, the methods include generating a library of test nucleic acid sequences, wherein each test nucleic acid sequence has a different nucleotide sequence for at least one of the regions of interest; screening the library for functional test nucleic acid sequences; sequencing the functional test nucleic acid sequences. In some embodiments, the sequencing comprises: performing a first polymerase chain reaction (PCR), wherein the first PCR provides a first PCR product comprising the least two regions of interest but does not include at least a portion of the intervening sequence; performing a ligation reaction, wherein the ligation reaction provides a first ligation product comprising the two regions of interest, wherein the two regions of interest are positioned less than 300 nucleotides apart; performing a second PCR, wherein the second PCR provides a second PCR product comprising the two regions of interest; sequencing the second PCR product and the two regions of interest. In some embodiments, the sequencing comprises next generation sequencing (NGS).

In some embodiments, the two regions of interest are positioned more than about 5, 10, 50, 100, 200, 300, 500, 1000, 1500, 2000, 2500, or 5000 nucleotides apart. In some embodiments, the two regions of interest are positioned more than about 300 nucleotides apart.

NGS sequencing methods are well known in the art, with a variety of platforms and chemistries. One non-limiting example includes the Illumina NGS sequencing methods.

Exemplary Advantages of Ribo-T v3

Ribo-T v3 features new tether RNA sequences (T1: 5′-AGUCAAUAA-3′ and T2: 5′-GACCUUCG-3′) as well as designed base pairs at the Tether-H101 junction. Ribo-T v3 exhibits up to a 58% improvement in orthogonal sfGFP translation and a 97% improvement in growth rate as well as a 77% improvement in lag time in SQ171 cells growing in M9 minimal media. Interestingly, cells supported by Ribo-T v3 in rich LB-Miller media exhibit comparable growth rates to those living on Ribo-T v2, but a 59% improvement in lag time. This is consistent with our evolution experiments which favor cells that emerge from stationary phase most quickly.

Additionally, we showcase Ribo-T v3's potential for expanding the chemical toolbox of orthogonal translation systems through the incorporation of a new non-canonical amino acid featuring a bulky side chain (DECP) into a peptide using an in vitro transcription and translation reaction supplemented with synthetic tRNAs charged with flexizyme.

Miscellaneous

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1. 3D-Structure-Guided Evolution of a Ribosome with Tethered Subunits

Abstract

RNA-based macromolecular machines, such as the ribosome, have functional parts reliant on structural interactions spanning sequence-distant regions. These features hamper the engineering potential of such machines because they limit evolutionary exploration of mutant libraries and confound 3D structure-guided design. To address these challenges, the inventors describe Evolink (evolution and linkage), a method that enables high-throughput evolution of sequence-distant regions in large molecular machines, and library design guided by computational RNA modeling to enable thorough exploration of structurally stable designs. To showcase the utility of this approach, the inventors evolved a tethered ribosome, which improves upon previous iterations by 58% in orthogonal protein translation and a nearly two-fold improvement in growth in minimal media. The Evolink approach enhances the engineering of macromolecular machines for new and improved functions with implications for synthetic biology.

Introduction

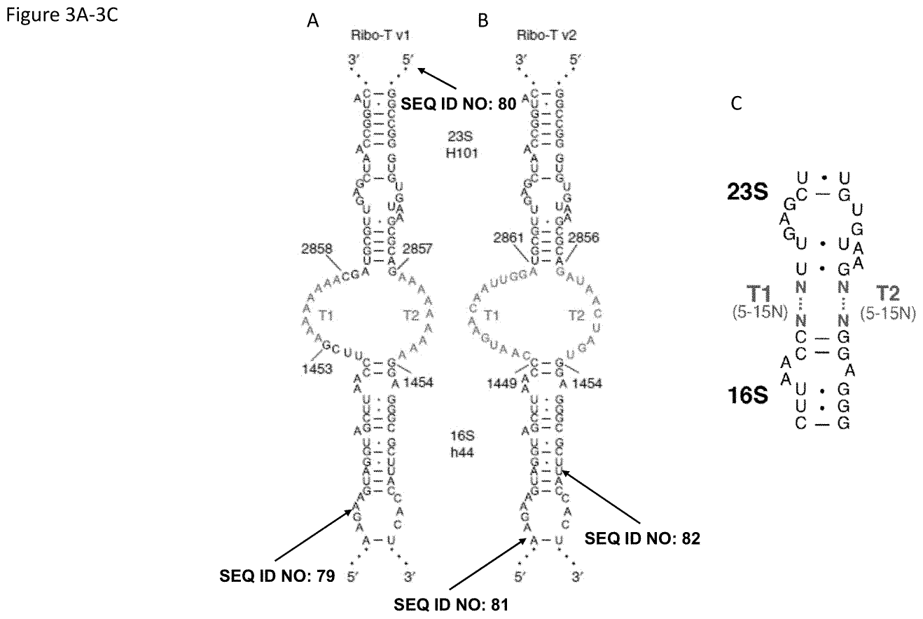

Directed evolution of RNA- and protein-based enzymes can elucidate principles of biological design and generate new catalytic activities for synthetic biology¹⁻⁸. Unfortunately, methods for directed evolution can be hindered by practical considerations. For example, the combinatorial space for evolution is immense (i.e., for an average protein of length 300 amino acids, there are a seemingly infinite number of theoretically possible amino acid sequences (˜20³⁰⁰)), and random mutagenesis alone cannot screen all possible variants⁹⁻¹². In addition, macromolecular machines often have complex tertiary structures that contribute to their function¹³, which bring residues that are distant in primary sequence close in three-dimensional space FIG. 3A. This limits the ability to recover in high throughput winning designs even when effective selections can be employed. Such practical limitations are exacerbated in large macromolecular machines, such as the bacterial ribosome, which has 3 ribosomal RNAs (rRNAs) comprising ˜4500 nucleotides (i.e., the 16S rRNA, 23S rRNA, and 5S rRNA) and 54 proteins^{1-4,8,9,14}.

Despite these challenges, directed evolution of the ribosome has emerged as a promising opportunity in chemical and synthetic biology^{1-5,7-9,14-21}. A major goal of ribosome evolution efforts is to repurpose the ribosome for diverse genetically encoded chemistries to create new classes of enzymes, therapeutics, and materials by selectively incorporating non-canonical monomers into peptides and proteins. While the natural ribosome works well for many noncanonical α-amino acids, there is poor compatibility with the natural translation apparatus for numerous classes of non-α-amino acids (e.g., backbone-extended amino acids (γ-, δ-, ε-, etc.)) leading to inefficiencies in incorporation^{1-4,22,23}.

Methods for engineering ribosomes have been developed to address these inefficiencies^{7,16,17,24,25}. In vivo, ribosome engineering methods have focused on the development of specialized ribosome systems. Recently, the advent of tethered ribosomes has made possible the first fully orthogonal ribosome-mRNA system in cells, where a sub-population of ribosomes are available for engineering and are independent from wild-type ribosomes supporting cell life¹⁸. Tethered ribosome systems have two key features. First, the anti-Shine-Dalgarno sequence of the 16S ribosomal RNA (rRNA) of the small 30S subunit can be mutated to function as orthogonal ribosomes that selectively initiate translation of orthogonal messenger RNAs (mRNAs) with mutated Shine-Dalgarno sequences^{19,26,27}. Second, the small and large subunits are covalently linked together FIG. 1B. In the first tethered ribosome system, termed Ribo-T, the core 16S and 23 S rRNAs were joined together to form a single chimeric molecule via helix h44 of the 16S rRNA and helix H101 of the 23S rRNA18. Importantly, by selecting otherwise dominantly lethal rRNA mutations in the large ribosomal subunit, Ribo-T was evolved to synthesize protein sequences that are inaccessible to the natural ribosome¹⁸. Since the initial discovery of Ribo-T and a subsequent stapled design¹⁵, new orthogonal Ribo-T/mRNA pairs as well as tether sequences have been optimized using directed evolution methods^{9,14}. Specifically, tether residues have been randomized in sequence but not in length⁹, or mutations to surrounding residues surrounding a fixed RNA linker (the J5/5a junction from the Tetrahymena group I intron) have been investigated¹⁴. Despite the improvement, the potential of tethered ribosome systems remains limited by their low activity.

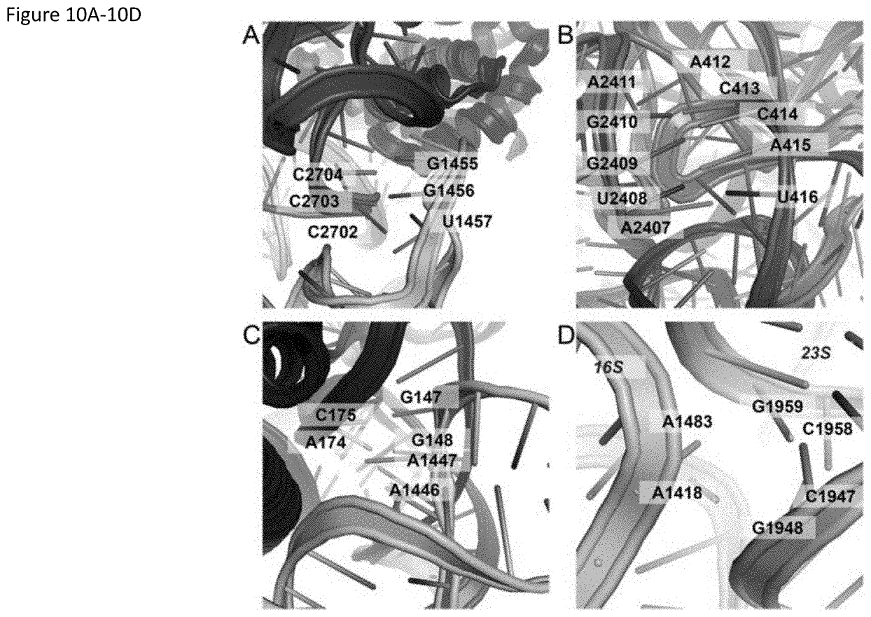

The untapped potential and existing inefficiencies of tethered ribosome systems motivate the need for new directed evolution-based approaches to engineer these systems for improving their activity. Previous works were limited in throughput in evaluating designs (e.g., 48 and 108 members were evaluated in two different efforts^{9,14}) due to their reliance on clonal isolation and functional testing. A bottleneck in these efforts has been that the regions of interest in the tethered ribosomes are separated by around 2,900 nucleotides (the length of the circularly permuted 23S rRNA18), and current readily available methods for next-generation sequencing are typically limited to overlapping read lengths of ˜300 nucleotides. While methods have been developed to address these shortcomings^{28,29}, they face limitations that hinder broad applications to macromolecular machines as large as the ribosome, which feature many examples of distantly sequence encoded, but physically interacting regions FIG. 10A-10D. Briefly, they rely on custom bioinformatic pipelines, barcoding strategies inherent to protein-based machines, or are limited in the distance between regions of interest²⁸⁺.

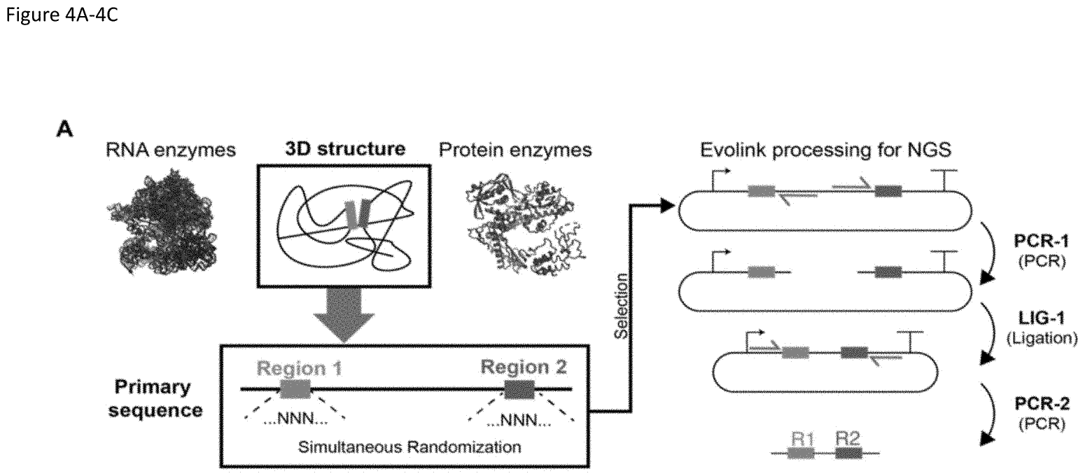

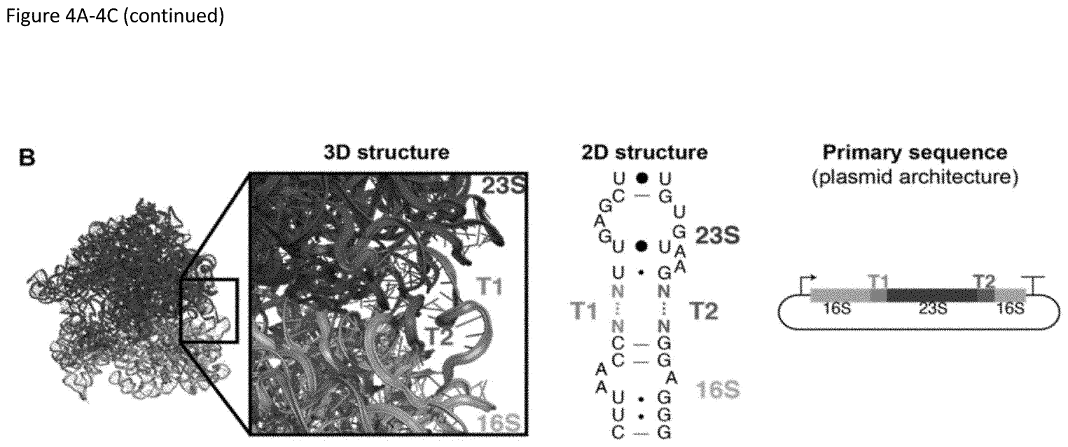

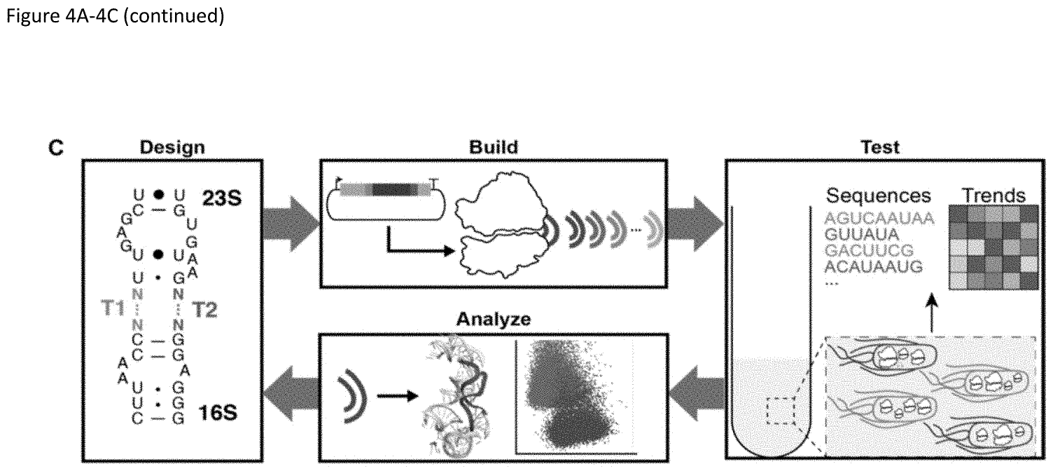

Here, to address existing limitations and facilitate evolution of ribosomes, we present a molecular biology technique called Evolink (evolution and linkage) FIG. 4A. Evolink connects two or more regions of nucleic acid sequence that are distant in primary space but close in 3D structure (in RNA or protein form) to enable next generation sequencing readouts of winning phenotypes. We apply Evolink to tethered ribosomes FIG. 4B and augment the method by integrating computational modeling with the design-build-test cycles of directed evolution to inform library design FIG. 4C. We use this integrated method to evolve tethered ribosomes for improved function by targeting the rRNA residues involved in connecting the 16S and 23 S rRNAs. We identify a newly evolved tethered ribosome (termed Ribo-T v3) that improves ribosome function nearly two-fold when supporting cellular growth in minimal media. Further, we demonstrate the compatibility Ribo-T v3 with non-canonical monomer incorporation in an in vitro protein synthesis reaction. The combination of Evolink with computational modeling allows for efficient evolution of macromolecular machines with complex structures, such as the ribosome, featuring regions distant in primary sequence but functionally linked in spatial proximity. We anticipate the Evolink approach will be valuable for future engineering of ribosomes and other macromolecular machines.

Results

Linking of Sequence-Distant Regions on a Single Next-Generation Sequencing Read

We aimed to develop a generalizable method, guided by computational design, for directed evolution of sequence-distant sites of macromolecular machines. As a model, we focused on evolving the tether sequences of covalently tethered ribosomes. To achieve our goal, we first developed the molecular biology methods needed, termed Evolink. Evolink is a three-step process that uses polymerase chain reaction (PCR), ligation, and a second PCR reaction to bring together sequence-separated regions of a plasmid into a single next-generation sequencing (NGS) read. This process is analogous to amplifying and closing the “backbone” of a plasmid, where the “insert” omitted from amplification is the RNA sequence separating the two regions of interest. Because Evolink relies on simple, general-purpose molecular biology (e.g., PCR and ligation), it can be adapted to any plasmid-encoded molecular machine FIG. 4A.

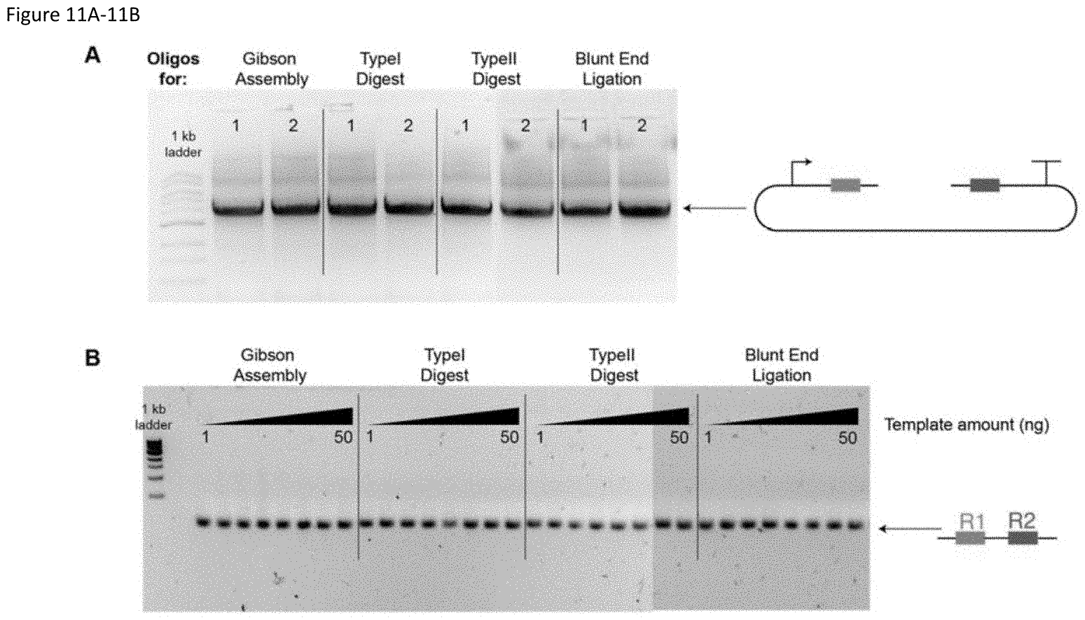

To start, we demonstrated the three key molecular biology steps of Evolink (termed PCR-1, LIG-1, PCR-2) FIG. 4A, right. Using a clonal plasmid sample encoding Ribo-T v29 FIG. 11A-11B, we initially carried out around-the-world PCR (PCR-1) with a high-fidelity polymerase (Q5 DNA Polymerase) using oligonucleotide primers specific to the plasmid. In our architecture, in which T1 is upstream (5′) of T2, the forward primer binds upstream of T2, and the reverse primer binds immediately downstream (3′) of T1, so the first set of primers for PCR-1 are “inside” the two regions of interest. The PCR-1 primers play two key roles. First, the sequence between each respective primer and region of interest (reverse primer-T1 and forward primer-T2 in this case) determines the length of the final amplicon for use in NGS. Second, the primers can encode compatible DNA sequences with either an overhang (for restriction enzyme-based or isothermal assembly31) or blunt ends to be used in the subsequent ligation step (LIG-1). We assessed the compatibility of PCR-1 with multiple primer sets that feature designed overhangs for Type I/II restriction enzyme digestion, 5′ phosphorylation for blunt-end ligation, or overlapping complimentary sequences for isothermal assembly. We found the first PCR step (PCR-1) to be successful with all four primer sets that featured different 5′ modifications (either phosphorylation or custom sequences) FIG. 11A-11B.

Following the first PCR, LIG-1 was carried out to cyclize the product of PCR-1 in a unimolecular ligation, proximally linking the previously distant regions. Prior to ligation, PCR-1 products that used primers compatible with restriction enzyme digests were processed with enzymatic digest and purification. Those that used 5′ phosphorylated primers or enzymatic digestion were purified and used in ligation with T4 ligase, and those which featured overlapping complementary sequences were ligated together using isothermal assembly³¹.

Finally, we carried out PCR-2 with a different set of primers to amplify the now-linked regions of interest. In this step, the primers are designed with the forward primer upstream of T1 and the new reverse primer downstream of T2, such that now the primers are “outside” of the regions of interest. The sequences between each respective primer and region of interest (forward primer-T1 and reverse primer-T2 in this case) contribute to the final amplicon length for sequencing. We designed primers such that the final amplicon product is ˜200 nucleotides in length and can be directly used in NGS library preparation. To demonstrate robustness, we tested the PCR-2 with four different ligation methods (Type I/II restriction enzyme digestion and ligation, blunt end ligation, and isothermal assembly), each with eight different input template amounts into the ligation (1, 2, 5, 10, 20, 30, 40, 50 ng). We observed successful generation of the desired amplicon for NGS for all 32 reactions tested FIG. 11A-11B. To reduce any possible biases, we moved forward with blunt-end ligation because it did not rely on any particular DNA sequence and proved successful at the minimum amount of template tested (1 ng).

Applying Evolink to Tethered Ribosomes

With the Evolink method in hand, we sought to apply it to develop mutant tethered ribosomes for improved activity, with a focus on tether design and evolution FIG. 4B. Specifically, we looked to improve upon the function of Ribo-T v232 by optimizing the tether residues for length and sequence composition. The guiding principle was to leverage the throughput of Evolink and post-facto structural modeling to evolve the RNA residues that make up the tethers. Central to our efforts was the iterative application of a design-build-test-analyze (DBTA) cycle FIG. 4C, in which multiple libraries can be tested, each library subsequently building upon results and analysis of the ones prior, to improve molecular function. This departs from previous efforts that carried out a single pass of library design, building, and selection/screening, which limits the breadth of the libraries to be tested. Our study was carried out with the notion that we would first start broadly, then through our DBTA cycles, test our hypotheses on tether design and narrow our search space with each cycle to arrive at an improved molecular machine. Because Evolink makes use of next-generation sequencing, our approach also allows for substantially larger sampling and screening of the solution space compared to past efforts.

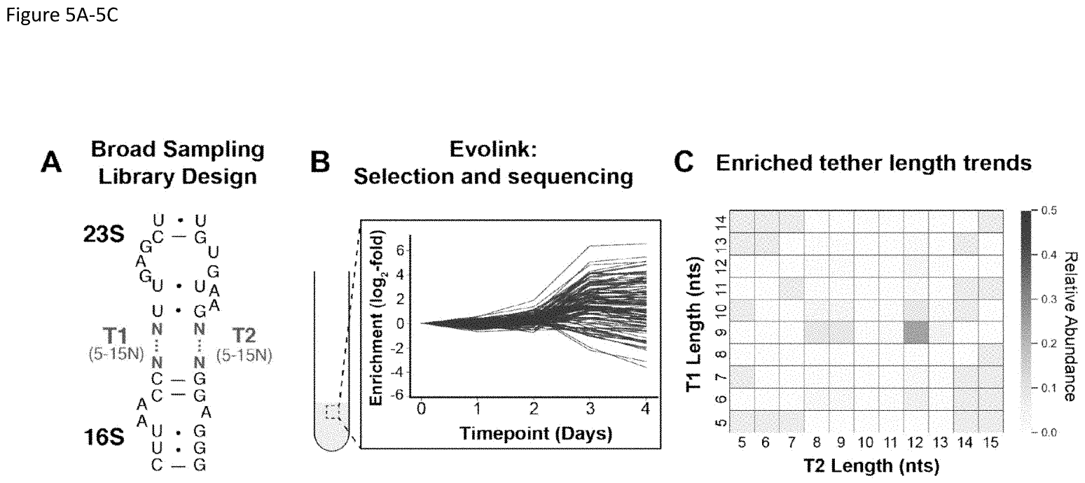

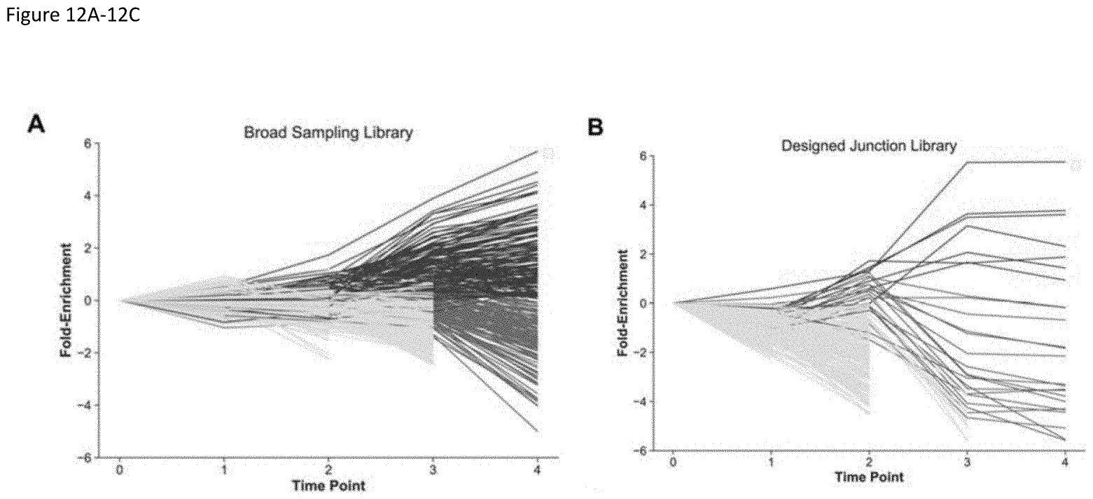

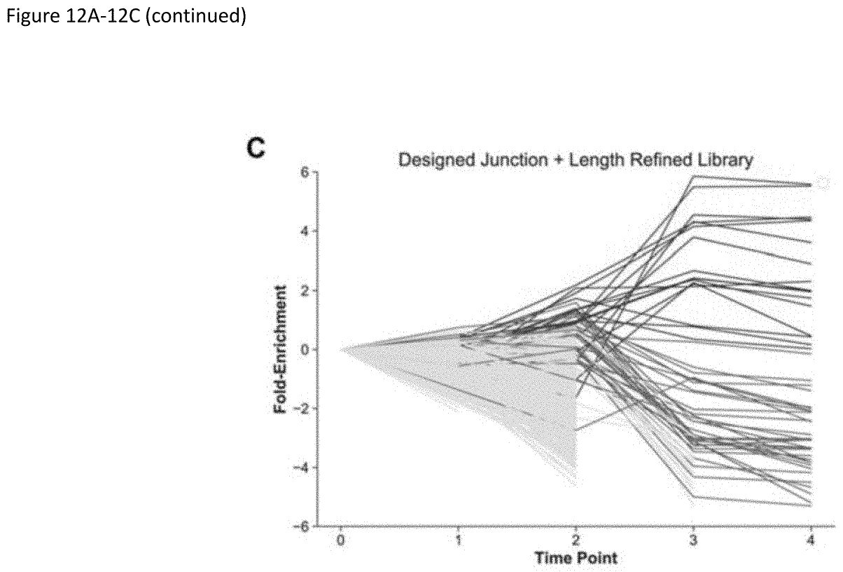

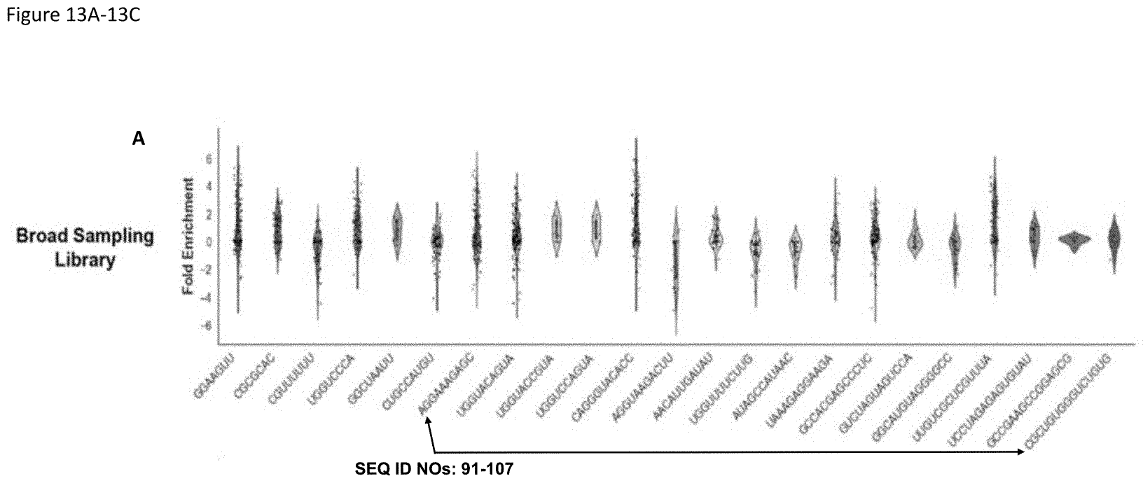

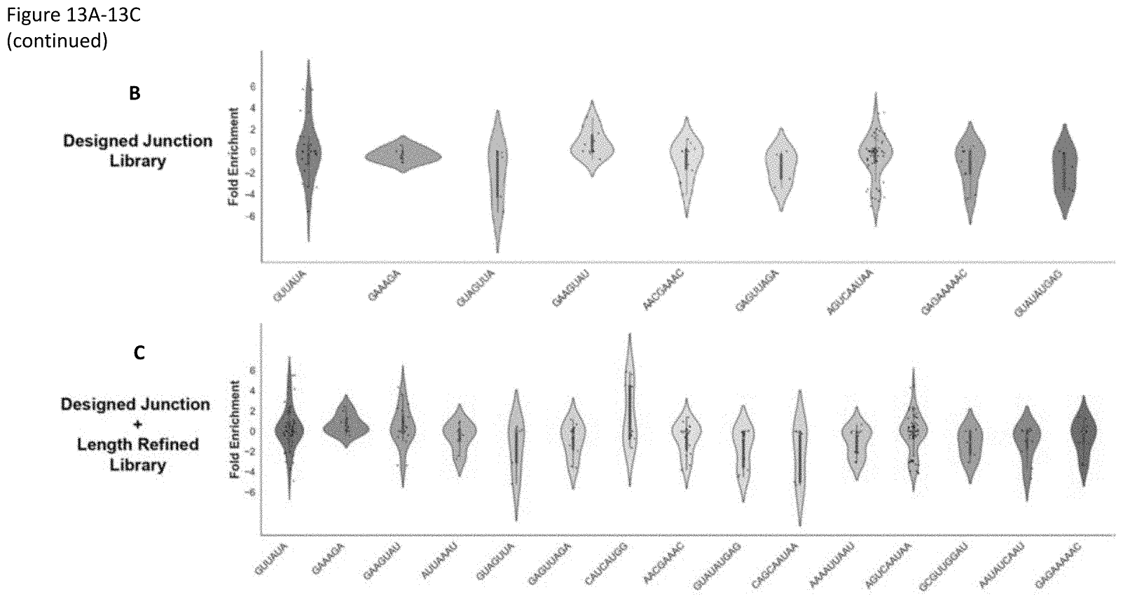

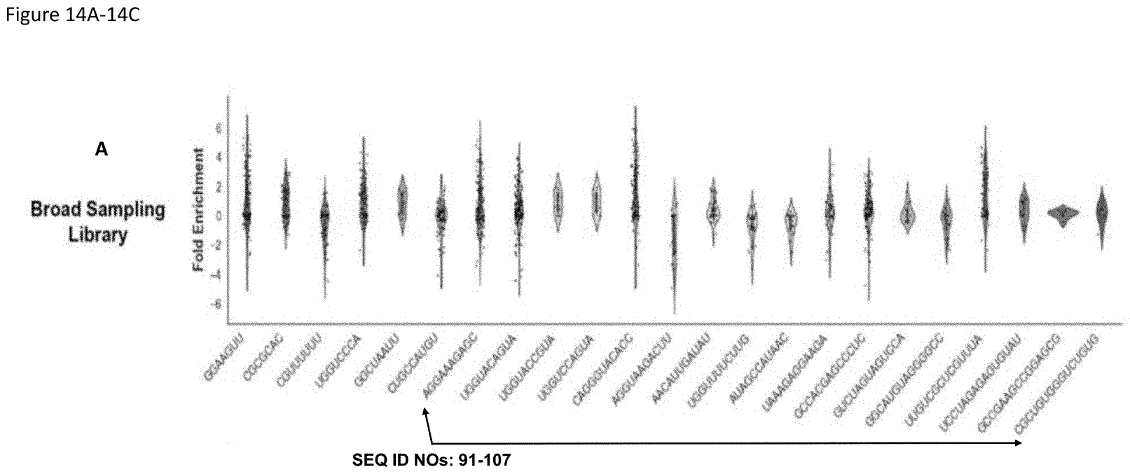

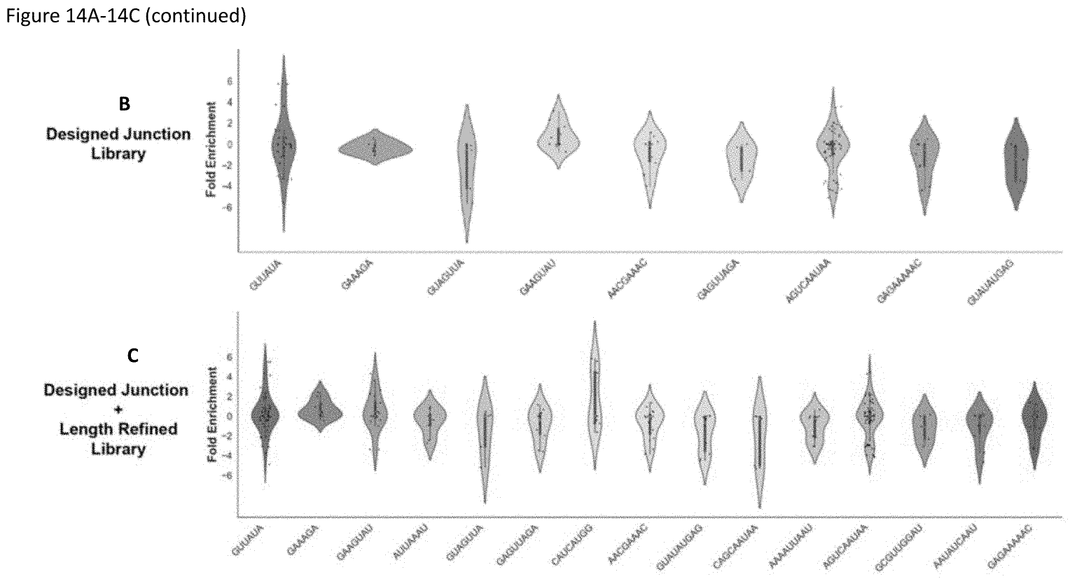

In the first library, we elected to broadly sample possible lengths and sequences of T1 and T2, with a degenerate library ranging from 5-15 nucleotides FIG. 5A. Following construction, the library of tether designs was cloned and transformed into an E. coli strain lacking rrn operons on the genome³³ and viable cells, which were growing exclusively off the tethered ribosomes, were identified by growth on agar plates^{9,18}. Resulting colonies were collected and selection was carried out in liquid culture FIG. 5B. By passaging cells in liquid culture for multiple generations (˜40 generations in this work), we hypothesized that faster growing mutants would become more enriched in the culture. Cells were subject to Evolink and analysis of subsequent NGS reads were carried out daily for four days. In the NGS reads, T1 and T2 sequences, which represent the two strands of RNA that make up the tether, were directly linked in a single amplicon, taking advantage of overlapping reads with high sequencing fidelity to improve our confidence in identifying pairwise interactions between the two regions. NGS analysis revealed a range of enrichments for many genotypes observed over the passaging time course FIG. 5B. Specifically, we observed enrichment (log 2-fold change) values between −5 to 6, and ˜1800 unique genotypes after the LB agar-based selection converging to ˜450 unique genotypes over the time course FIG. 5B, FIG. 12A-12C. Two key features emerged from these data. First, the same T1 sequences paired with multiple T2 sequences FIG. 13A-13C. For example, T1: 5′-CAGGGUACACC-3′ paired with T2: 5′-CCCAUUCA-3′, 5′-AUUCACUUGG-3′, and 5′-CGACGAGCG-3′ to yield enrichment values of 5.69, 2.17, and −1.5, respectively. These data suggest that contributions of the two tether sequences to overall ribosome assembly and function depend on each other and are not simply additive. Second, we observed a trend in the sequencing data towards specific optimal tether lengths, converging upon a length of 9 nucleotides for T1 and 12 nucleotides for T2 FIG. 5C.

Structural Fragility of the Tether-H101 Junction

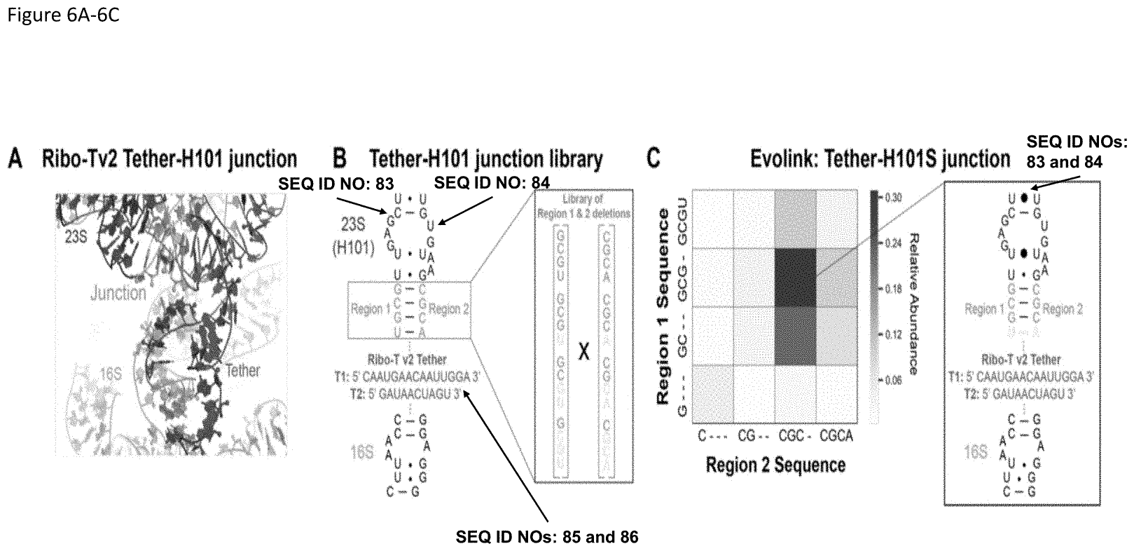

Based on previous literature that showed stapled ribosome function is sensitive to the connection between the tether and 23S rRNA residues¹⁴ (henceforth referred to as the Tether-H101 junction), we wondered if the Tether-H101 junction would also be significant in the Ribo-T design context^{9,18} FIG. 6A. To explore this hypothesis, we next fixed the tether identity according to the Ribo-T v2 sequence⁹ and constructed a library that consisted of every possible combination of base deletions in the Tether-H101 junction region FIG. 6B. This allowed us to approach the problem from an unbiased perspective, without preexisting assumptions on whether these residues indeed exist in a base-paired helical form or in another rearranged architecture. Following library construction, we again tested for the ability of these library members to support growth in the SQ171 strain. Evolink results on this library converged to 5′-GCG-3′ and 5′-CGC-3′ in Region 1 and Region 2, respectively, revealing that base changes in the Tether-H101 junction indeed affect ribosome function FIG. 6C. These results suggested that the folding behavior of this junction may have a significant influence on both tethered ribosome structure and function.

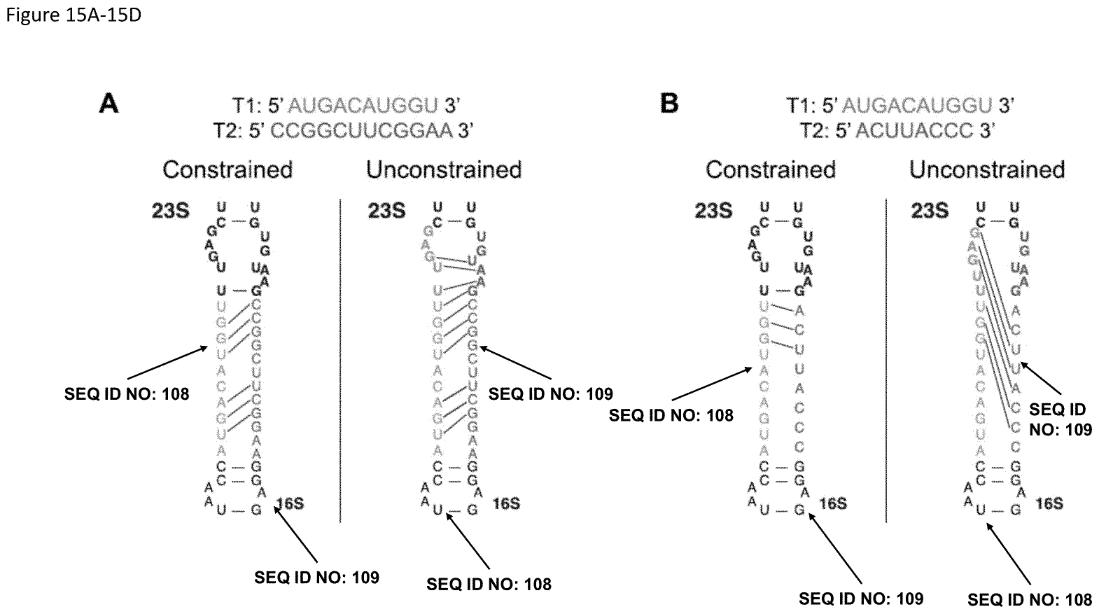

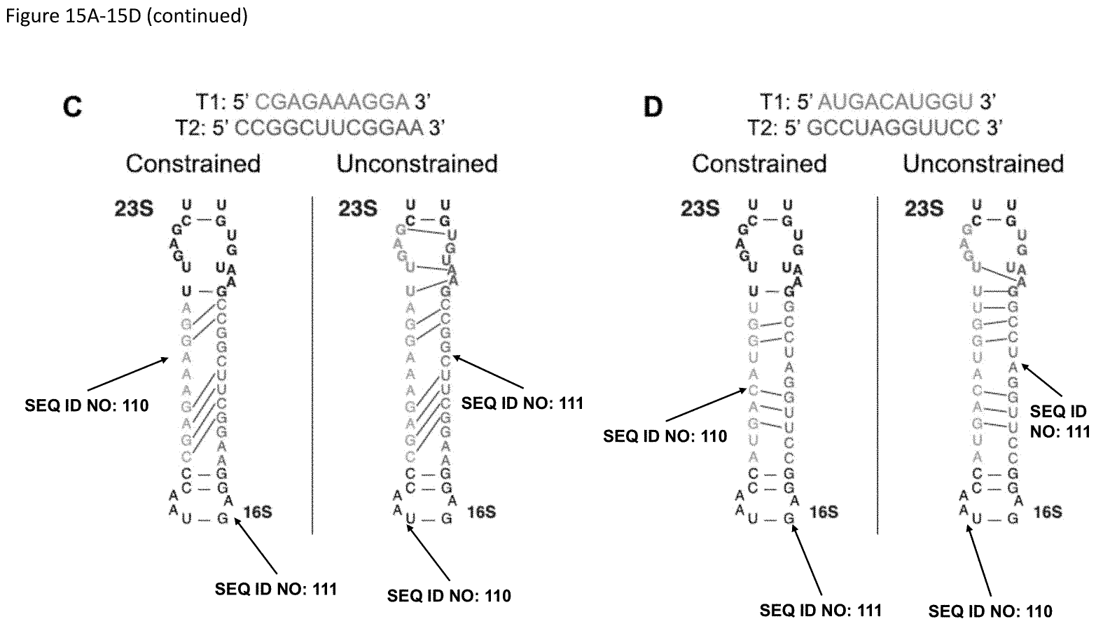

To further test and understand this hypothesis, we turned to computational modeling to gauge structural stability of the Tether-H101 junction FIG. 15A-15D. The key idea was to use modeling (secondary structure modeling with ViennaRNA34 and tertiary structure modeling with Rosetta FARFAR235) to understand possible structural features that may contribute to improved tether RNAs and overall ribosome function, and use those insights to inform subsequent library design. First, we used RNAcofold to conduct secondary structure predictions on the four most prevalent tether sequences that emerged from the Broad Sampling Library (e.g., a 10 nucleotide (nt)/12 nt tether, T1: 5′-AUGACAUGGU-3′ and T2: 5′-CCGGCUUCGGAA-3′) to assess the degree to which each tether's structure was dependent on its structural context FIG. 15A-15D. If the tether's structure is perfectly independent of the surrounding residues, the same base-pairing would be observed regardless of surrounding residues including in the RNAcofold analysis. To test this, we computed the minimum free energy secondary structure of the tether under two different conditions. The first, ‘unconstrained’ calculation, allowed the adjacent 23S rRNA junction (Helix 101 in the wild-type ribosomal 23S rRNA) to ‘re-fold’ rather than constraining it to assume the base pairing observed in experimental structures of the E. coli ribosome36. In the second, ‘constrained’ calculation, the 23S rRNA junction residues are instead required to assume that experimental base pairing. For three of four tethers, we observed the same tether base pairs in the constrained and unconstrained structures, but the adjacent 23S junction only maintained its wildtype structure in one case FIG. 15A, C, D. For the remaining tether, significantly different RNA secondary structures were observed between the ‘constrained’ and ‘unconstrained’ models FIG. 15B.

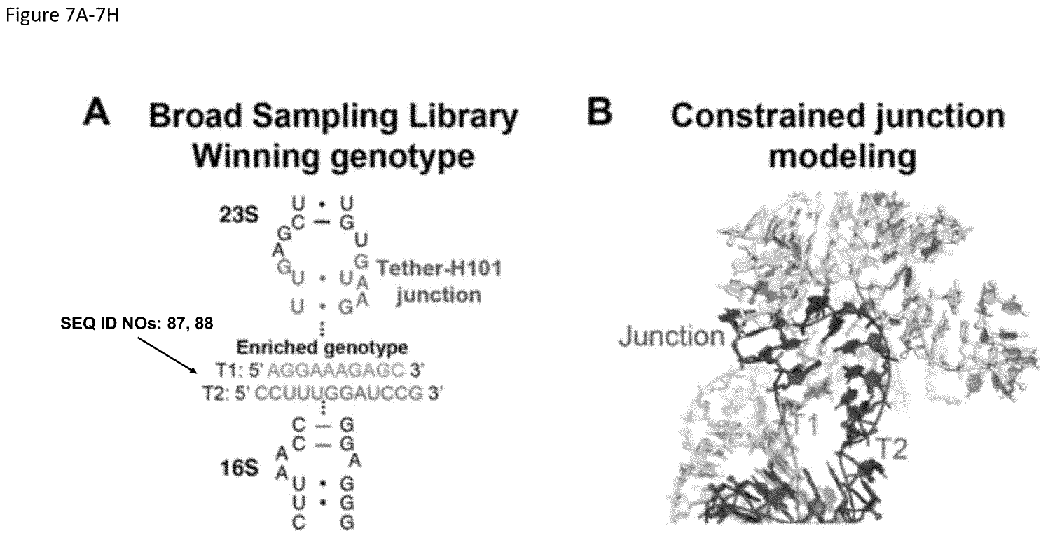

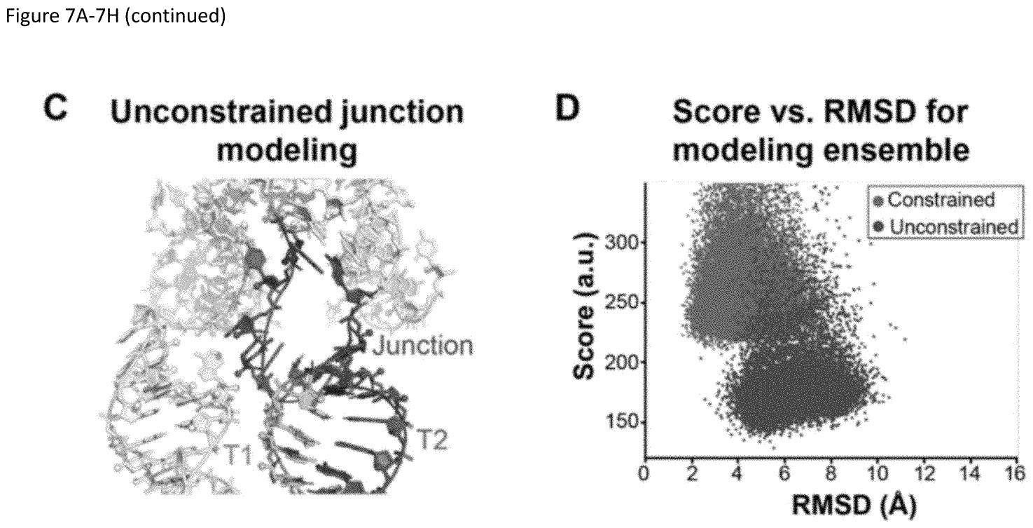

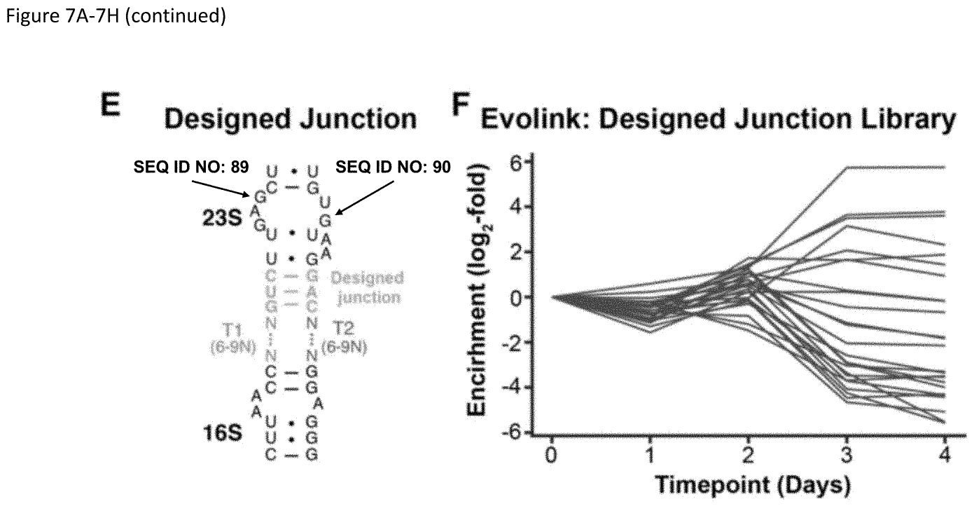

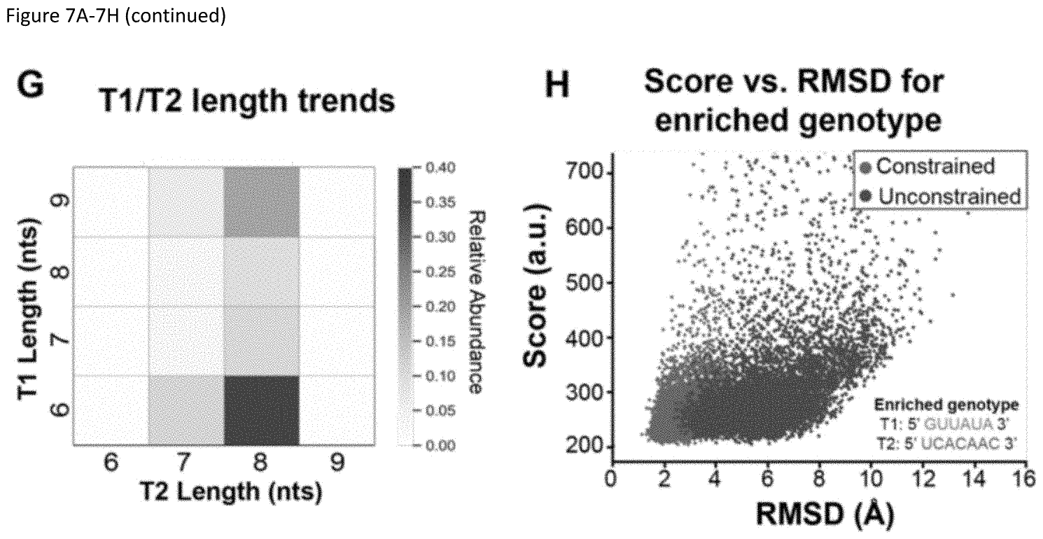

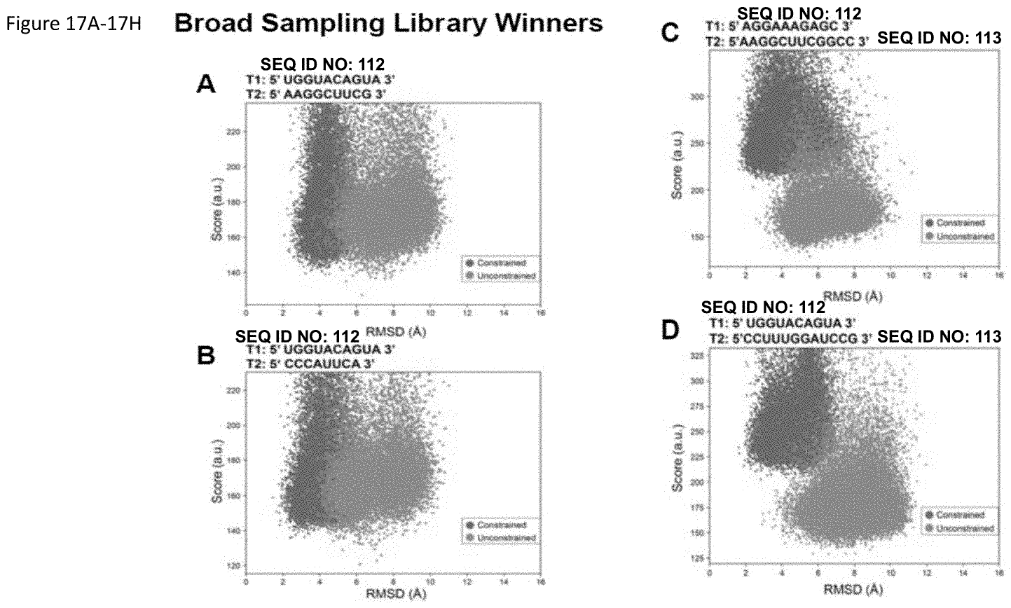

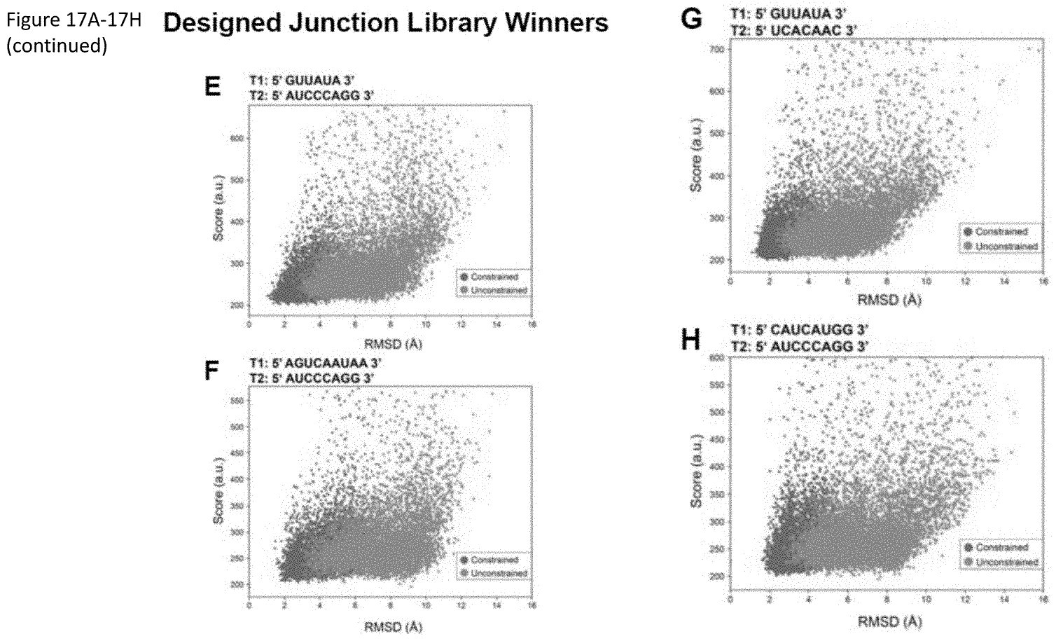

We conducted 3D modeling of these tethers to augment our understanding FIGS. 7A-D and FIG. 17A-D. Specifically, we used Rosetta's RNA fragment assembly code35 to model analogous constrained and unconstrained states of the tether with FARFAR2 FIGS. 7B and 7C, respectively, and FIG. 17A-D. For each tether, the constrained and unconstrained simulations resulted in significantly different structures and energy distributions compare FIGS. 7B, 7C; see also FIG. 7D, FIG. 17A-D, suggesting that the Tether-H101 junction may not be particularly stable. Our results from investigating the Tether-H101 junction, both experimentally and computationally, led us to reinforce the structure of the Tether-H101 junction, as well as to optimize tether length and sequence together in subsequent rounds of directed evolution.

Evolink and Computational Validation of a Designed Tether Library

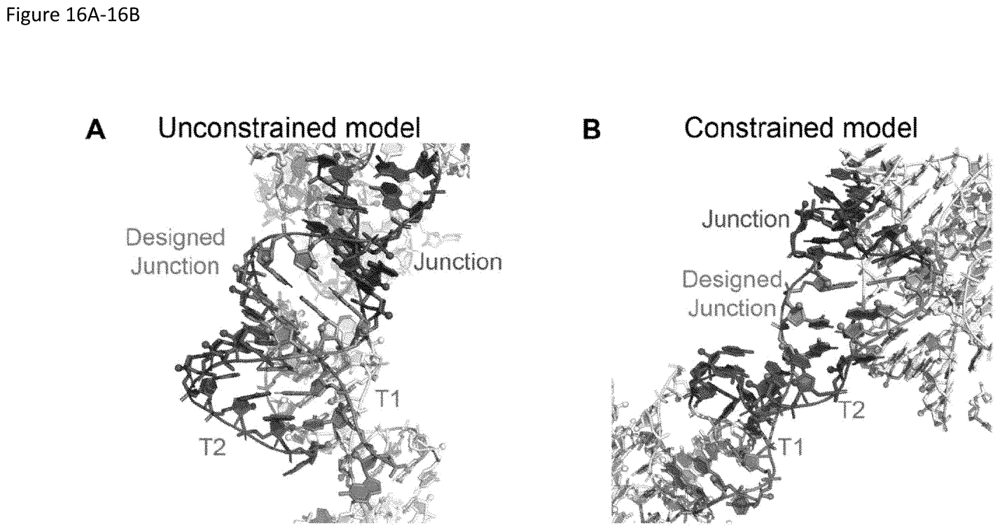

With the range of tether lengths informed by the Broad Sampling Library and the designed base pairs at the Tether-H101 junction, we next performed Evolink on a tether library followed by 3Dstructure analysis. The library featured 6 to 9 random nucleotides for both T1 and T2 regions, with the addition of three synthetic base pairs at the Tether-H101 junction to encourage its formation and increase the independence of tether folding from junction folding FIG. 7E. Selection and analysis were carried out as described above (over four time points/days)[FIG. 7F. Tether lengths converged to a length of 6 and 8 nucleotides for T1 and T2, respectively, with the winning sequence being T1: 5′-GUUAUA-3′ and T2: 5′-AUCCCAGG-3′ FIG. 7G. Post-facto modeling of select highly enriched genotypes as described previously (see Structural fragility of the Tether-H101 junction) revealed improved agreement between constrained and unconstrained conditions compared to the Broad Sampling Library FIG. 7H, FIG. 17E-H. Most notably, models revealed predicted base pairing in the designed junction residues in both the constrained and unconstrained models, as well as predicted base pairing in the Tether-H101 junction compared to the Broad Sampling Library winner compare FIG. 16A-B with FIG. 7B-C.

Clonal Isolation of Enriched Genotypes and Test of Orthogonal Protein Synthesis

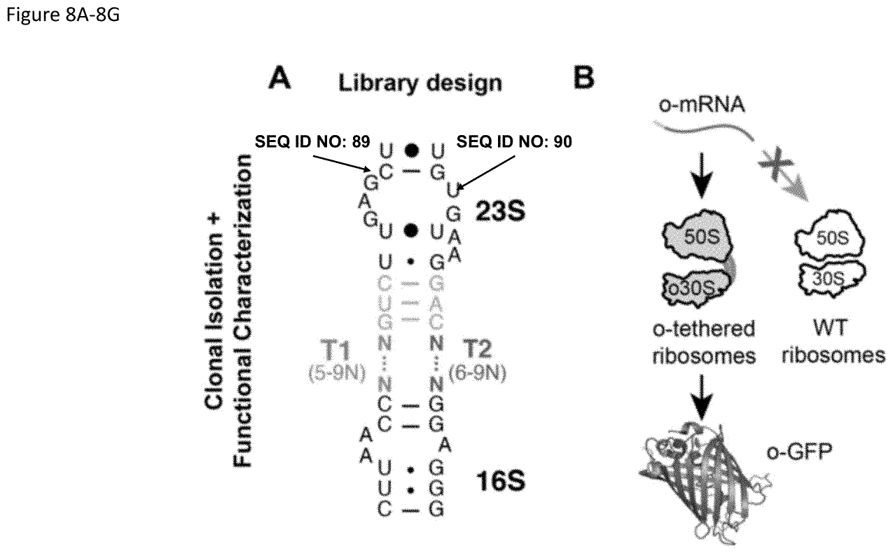

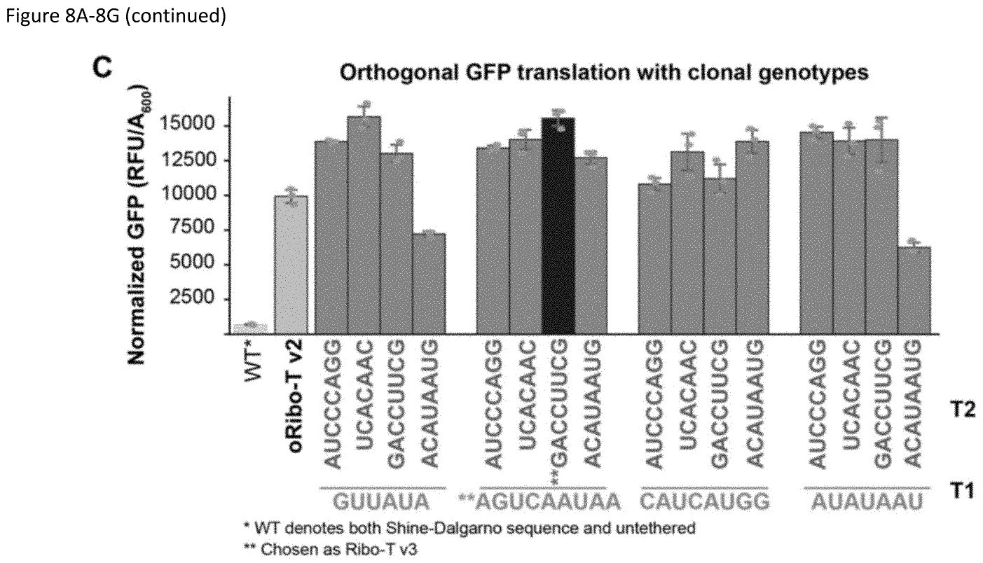

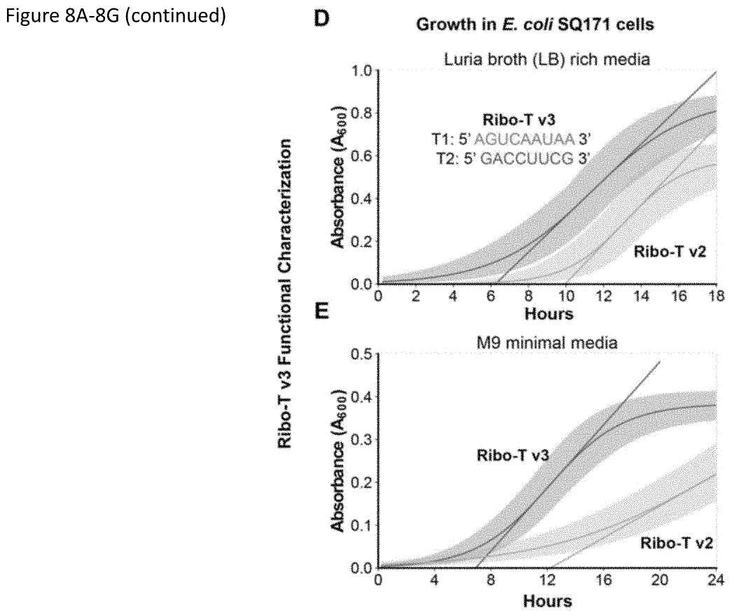

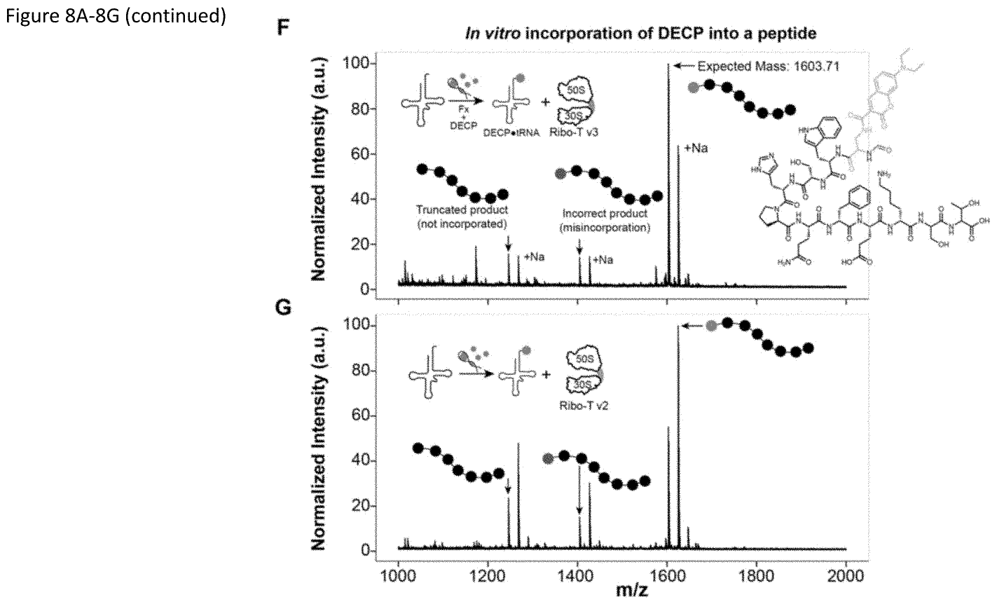

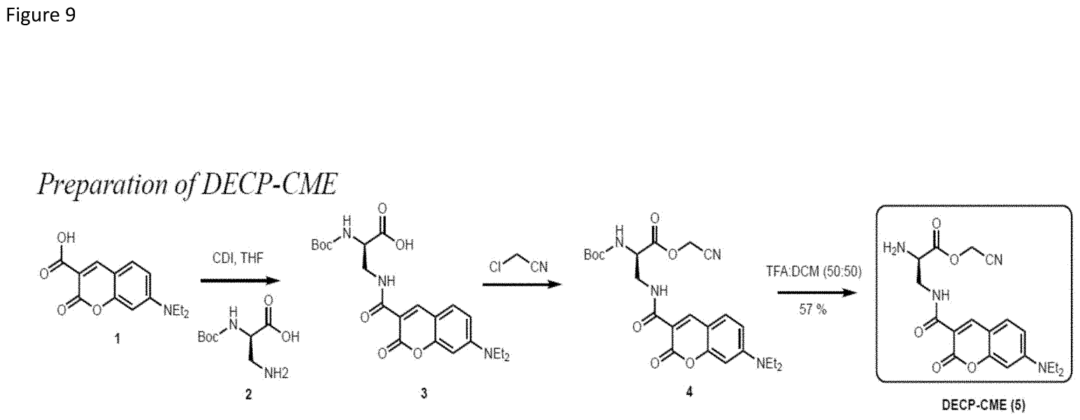

We then carried out a final round of randomized library building and selection. The goal of this selection was to identify candidates for clonal isolation and characterization of improved tethered ribosome genotypes. The library combined the lessons learned from our three previous libraries. First, we tested tether lengths ranging from 5 to 9 nucleotides for T1 and 6 to 9 nucleotides for T2 based on the previous round of Evolink converging to 6 and 8 nucleotides for T1 and T2, respectively FIG. 8A. Second, the library featured a designed Tether-H101 junction, which was reinforced by base pairs that we hoped would contribute to improved structural stability in the tethers. Evolink was carried out to identify enriched pairs of sequences encoding T1 and T2 FIG. 12A-C. Of the highly enriched genotypes, we sought to more explicitly test if T1 and T2 sequences displayed cooperativity as we had previously observed enrichment of specific combinations between T1 and T2 sequences during evolution FIG. 13A-C.

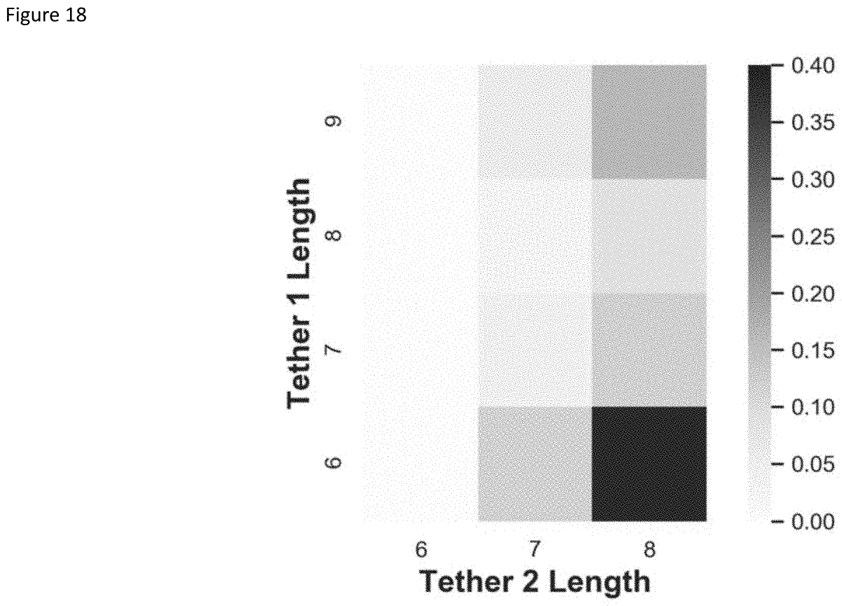

To test this cooperativity hypothesis and isolate a final winning genotype, we built 16 individual genotypes from the final library by combining the top 4 enriched sequences for the T1 and T2 regions from this round of Evolink and tested the combinations individually for their ability to carry out orthogonal superfolder GFP (sfGFP) synthesis compared to a previously improved orthogonal tethered ribosome, oRibo-T v2 FIG. 8B, 8C. The measurement of custom (orthogonal) translation output is a unique application for tethered ribosomes and an important measure of their function. In this experiment, the anti-Shine-Dalgarno of the tethered ribosome's small subunits are mutated to selectively translate mRNAs (encoding sfGFP) with correspondingly mutated Shine-Dalgarno sequences. Of the 16 tested genotypes, 14 T1/T2 pairs outperformed oRibo-T v2 in orthogonal GFP synthesis FIG. 8C, highlighting that our evolutionary strategy had worked to improve tethered ribosome function. Further, we observed combinatorial behavior amongst the 16 individual genotypes tested: as an extreme example, depending on the paired T1, the sequence T2: 5′-ACAUAAUG-3′ could perform 30% better than oRibo-Tv2 or 30% worse FIG. 8C, supporting the hypothesis that the tethers interact with functional consequences. The two highest performing tether genotypes were 1) T1: 5′-GUUAUA-3′ and T2: 5′-UCACAAG-3′; and 2) 5′-AGUCAAUAA-3′ and T2: 5′-GACCUUCG-3′, which each showed increased orthogonal protein synthesis over Ribo-T v2.0 by 56% and 58%, respectively FIG. 8C. Of these, we chose further characterization for T1: 5′-AGUCAAUAA-3′ and T2: 5′-GACCUUCG-3′, which we termed Ribo-T v3. The choice of this genotype was further supported by enrichment trends observed during selection which suggested a length of 8 nucleotides for T2 was more broadly enriched compared to a T1 length of 6 FIG. 18.

Functional Characterization of Ribo-T v3

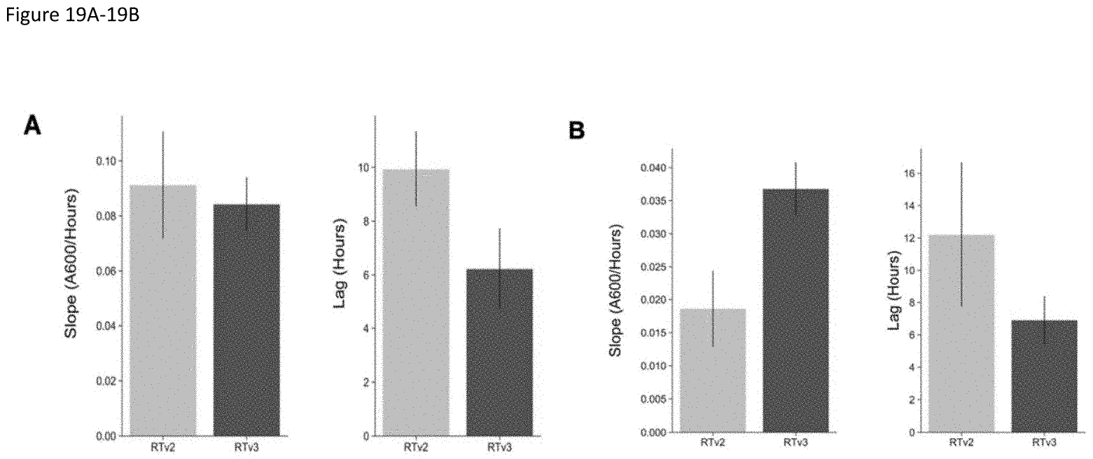

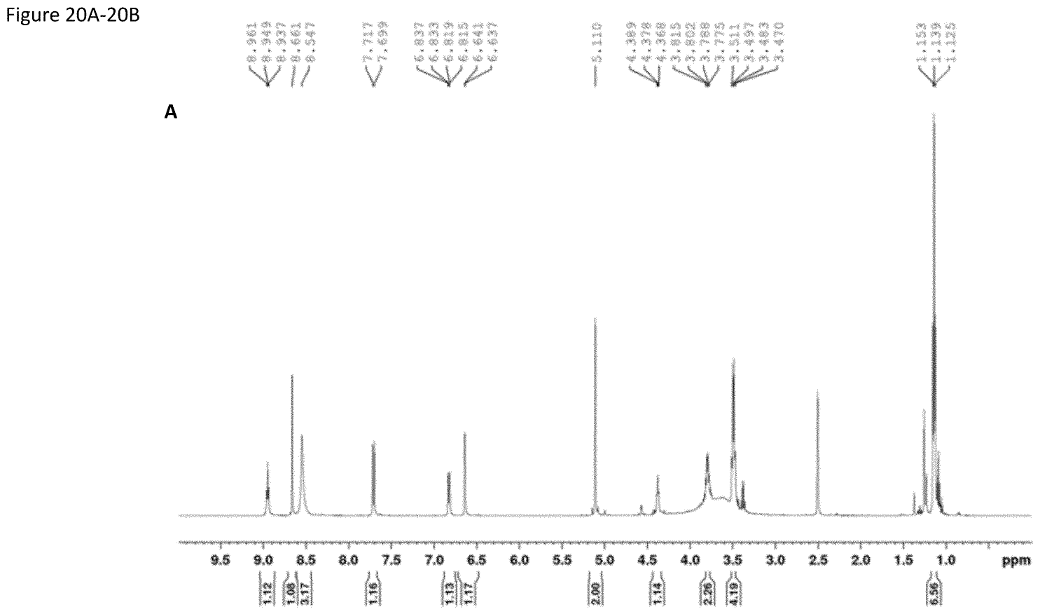

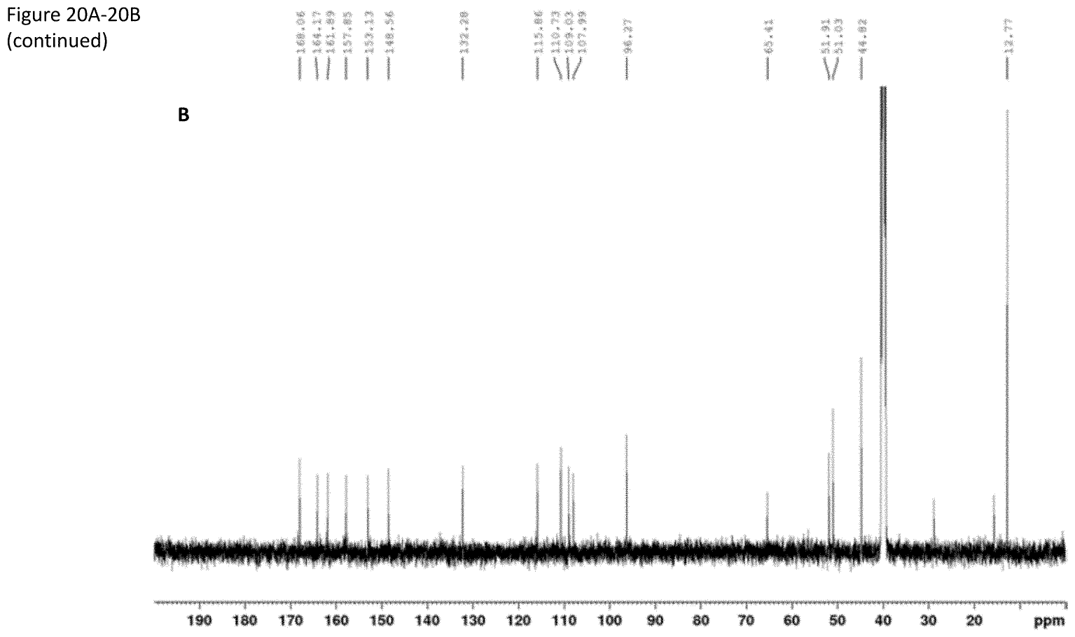

We next tested the ability of Ribo-T v3 to support cellular life in the SQ171 strain as a general measure of ribosome function9,18. We compared growth rates of cells supported by Ribo-T v3 and Ribo-T v2 on both minimal M9 media as well as rich LB-Miller media FIG. 8D. This revealed improved growth characteristics for cells growing on Ribo-T v3 especially in minimal M9 media as well as rich Luria Broth (LB) media FIGS. 58 & 8E, FIG. S19A-B. Notably, although doubling times in LB media were equal within error, cells growing on Ribo-T v3 exhibited a 97% improvement in doubling time in M9 media. Additionally, SQ171 cells living on Ribo-T v3 exhibited 59% and 77% improvements in lag time for LB and M9 media, respectively FIG. 19A-B. Interestingly, this suggests that differences between Ribo-T v2 and Ribo-T v3 extend beyond ribosome function at the molecular scale, but also has implications at the phenotypic level when considering coordination with other cellular machinery during the process of cellular growth. Considering that evolution for Ribo-T v3 was carried out in LB media, improvements of cellular growth on Ribo-T v3 over Ribo-T v2 in minimal M9 media as well as improvements in orthogonal protein yield suggest that evolutionary advantages in fitness can extend to multiple contexts.

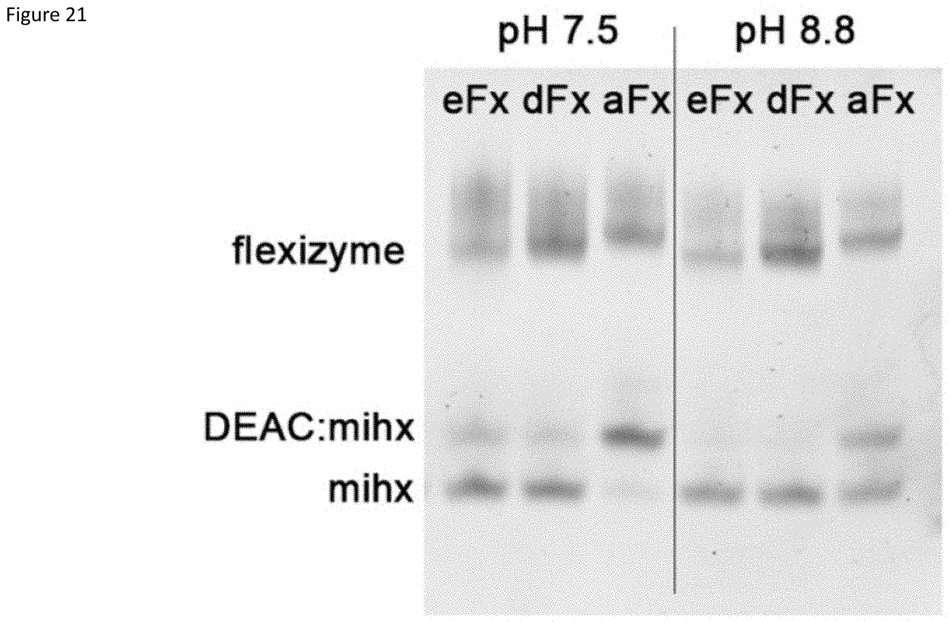

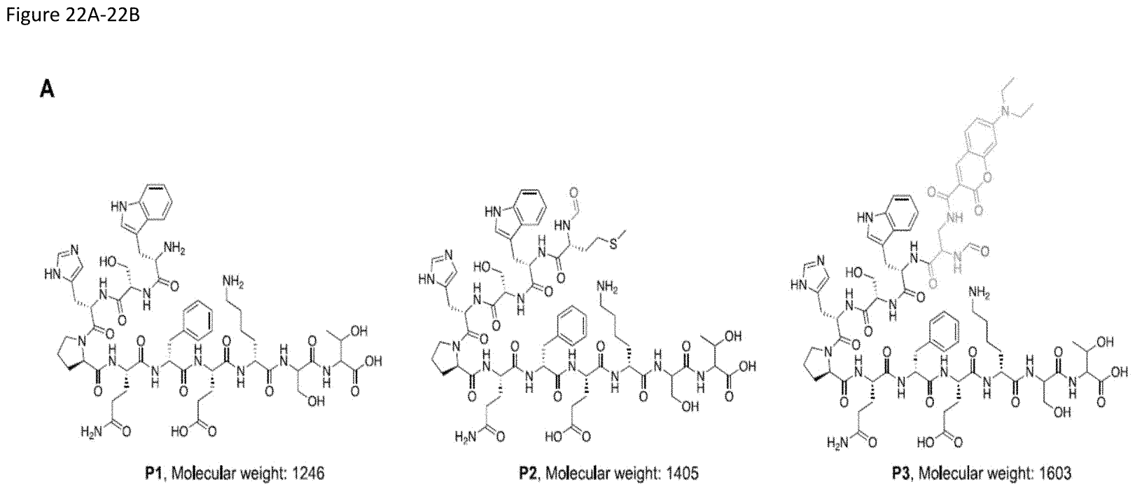

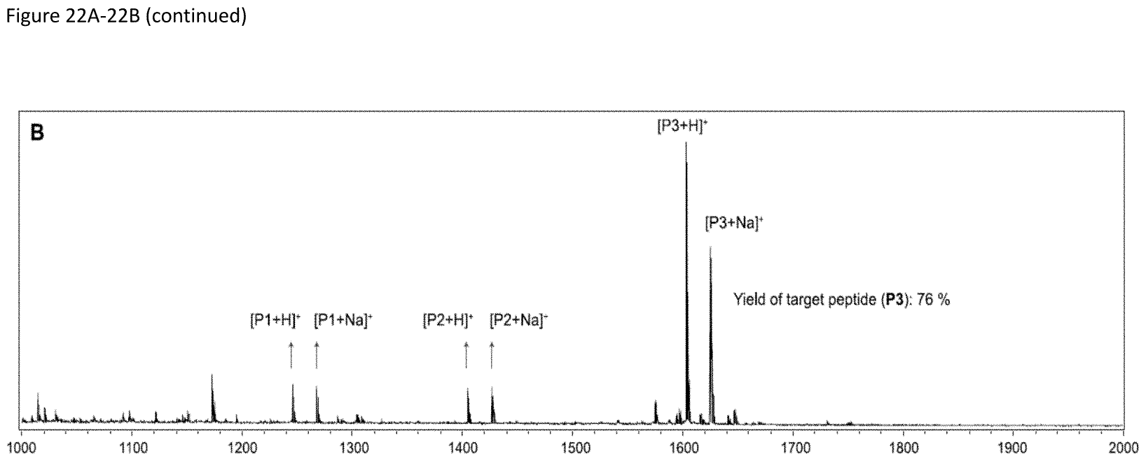

Towards this vision of genetic code expansion with tethered ribosomes, we tested the ability of Ribo-T v3 to incorporate a non-canonical amino acid into a peptide. The idea was not to engineer Ribo-T v3 further to be better than a natural ribosome at incorporating non-canonical amino acids, but rather to show that oRibo-T was compatible with applications geared towards expanding the chemistry of life^{1-4,14,23}. We chose a non-canonical L-α-amino acid ((R)-2-amino-3-(7-(diethylamino)-2-oxo-2H-chromene-3-carboxamido)propanoic acid, DECP) featuring a diethylamino coumarin group on its sidechain. The monomer, which features a bulky side chain, has not yet been shown to be incorporated into a peptide ribosomally, and thus presented a new and attractive target to showcase Ribo-T v3's ability to expand the chemical biology toolbox of engineered translation machinery. For demonstration purposes, and since evolved aminoacyltRNA synthetases do not exist for this monomer, we used a cell-free transcription and translation platform based on the PURExpress system³⁷⁻³⁹. In this platform, the monomer DECP was charged onto tRNAfMet(CAU) using a flexizyme³⁸ FIG. 21, and added to the PURExpress reaction with a sample of Ribo-T v3 or Ribo-T v2 purified from cells FIG. 8E. Mass spectrometry analysis revealed that DECP was successfully incorporated into the N-terminus of a peptide by both Ribo-T v3 and Ribo-T v2 FIGS. 8F & 8G, FIG. 21. We observed improved incorporation of DECP by Ribo-T v3 compared to Ribo-T v2 based on less prominent peaks of mis-incorporated or truncated products observed in MALDI-MS. These results suggest that Ribo-T v3's improved ribosome function may be applied to genetic code expansion.

### Discussion

In this work, we present an improved tethered ribosome platform, termed Ribo-T v3, evolved from the previous state-of-the-art (Ribo-T v2). Key to our effort was the development of Evolink, a technique for evolving regions in macromolecular machines far apart in primary sequence but proximal (and potentially functionally linked) in three-dimensional space. Evolink uses widely available molecular biology protocols (PCR and ligation) to link together distant sites of a plasmid in a single next-generation sequencing (NGS) read, alleviating previous limitations to ribosome evolution enforced by short NGS read lengths (˜300 nucleotides). We carried out four iterations of our design-build-test-analyze directed evolution experiment, featuring library designs informed by NGS results as well as structural modeling. Libraries explored simultaneous variation of tether sequence and length, as well as interaction between the tether and its junction with H101, culminating in design of a library that yielded Ribo-T v3.

Ribo-T v3 features new tether RNA sequences (T1: 5′-AGUCAAUAA-3′ and T2: 5′-GACCUUCG-3′) as well as designed base pairs at the Tether-H101 junction. Ribo-T v3 exhibits up to a 58% improvement in orthogonal sfGFP translation and a 97% improvement in growth rate as well as a 77% improvement in lag time in SQ171 cells growing in M9 minimal media. Interestingly, cells supported by Ribo-T v3 in rich LB-Miller media exhibit comparable growth rates to those living on Ribo-T v2, but a 59% improvement in lag time. This is consistent with our evolution experiments which favor cells that emerge from stationary phase most quickly. Additionally, we showcase Ribo-T v3's potential for expanding the chemical toolbox of orthogonal translation systems through the incorporation of a new non-canonical amino acid featuring a bulky side chain (DECP) into a peptide using an in vitro transcription and translation reaction supplemented with synthetic tRNAs charged with flexizyme. Looking forward, we predict that Ribo-T v3 will accelerate new advances in orthogonal translation systems to expand the palette of genetically encoded chemistries^{9,14,16,40}. Moreover, we expect Evolink will advance directed evolution efforts, especially those for large macromolecular machines, for synthetic biology.

Materials and Methods

Library Construction

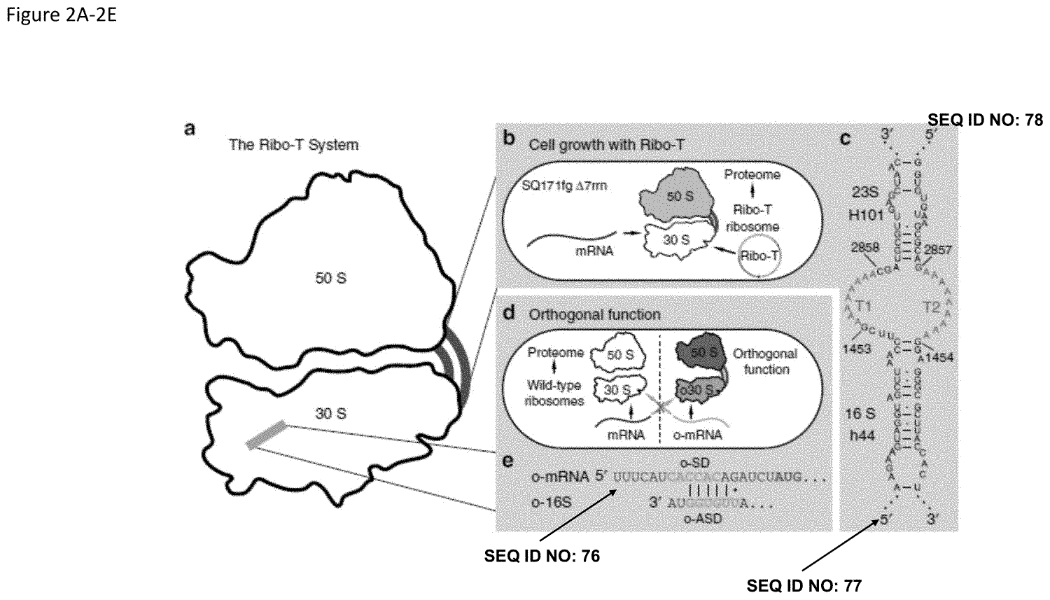

Plasmid libraries of Ribo-T tethers were generated using polymerase chain reaction (PCR) with the plasmid encoding Ribo-T v2.09, as the template. Oligonucleotides (IDT, USA) encoding degenerate bases (Ns) in place of the tethers were used to amplify the insert which includes both tethers and the 23S rRNA (referred to as the insert) [FIG. 1C]. For the Tether-23S junction, oligos encoded deletions in the specified region, not degenerate bases [FIG. 2E]. Another pair of oligos amplified the remainder of the plasmid (referred to as the backbone) [Table S1].

Resulting amplicons were purified using the Omega Cycle-Pure kit (Omega Bio-Tek), then digested with DpnI (NEB) to remove the template. The insert and backbone were ligated using isothermal DNA assembly31, and transformed into POP2136 cells via electroporation. Post-transformation, the cells were recovered in 800 μL of SOC at 30° C. for 90-120 minutes, then plated on LB-agar plates containing 100 μg/mL carbenicillin. The plates were incubated at 30° C. for 16-18 h until colonies appeared. All colonies were scraped from the agar plates and plasmid extraction was performed using a Zymo-PURE Midiprep II kit (Zymo Research).

Selection of Tethered Ribosomes

The libraries of Ribo-T tethers were transformed into SQ171 cells lacking chromosomal ribosomes32. 100 ng of the plasmid library was transformed into 50 μL of SQ171 cells via electroporation, then recovered with 500 μL SOC at 37° C. with shaking at 250 rpm for 2 h. After, another 1.5 mL of SOC was added to the cells and the final 2 ml culture was brought to 100 μg/Ml carbenicillin and 0.25% sucrose. These cells were then incubated at 37° C. with shaking at 250 rpm for 16-18 h. After incubation, cells were plated onto LB-agar plates containing: carbenicillin (100 μg/mL), sucrose (5% w/v), and erythromycin (250 μg/mL) and incubated at 37° C. for 20-24 h until colonies appeared. Colonies were then washed from the agar plates with LB containing 100 μg/mL carbenicillin (˜5 mL of LB-carbenicillin per 100 mm petri dish) and grown to saturation at 37° C. with 250 rpm shaking. 1 mL of the solution was reserved and plasmids were extracted using the Zymo-PURE Miniprep kit (Zymo Research). The saturated culture was then subject to passaging over 4 days in LB containing 100 μg/mL carbenicillin, and plasmids were extracted each day for sequencing.

Preparation of Amplicons for Next-Generation Sequencing

Plasmids extracted from selection cultures were linearized using PCR and purified using the Omega Cycle-Pure kit. 20 ng of the purified product was then used in a 20 μL ligation reaction containing T4 ligase (NEB) and the appropriate accompanying buffer. After incubation at 37° C. for 2 h, 2 μL of the ligation reaction was used directly in a 20 μL PCR with 15 cycles of amplification, which generated the amplicon for next-generation sequencing. The resulting product was then purified and prepared for next-generation sequencing using the NEBNext Ultra II DNA Library Prep kit (NEB). The resulting library was run on a MiSeq (Illumina) using a 150-cycle MiSeq Reagent Kit v3 (Illumina).

Analysis of Next-Generation Sequencing Results

Paired end reads from Illumina sequencing were assembled using PANDASeq39. Reads that had coverage (number of redundant reads) of less than ten were filtered and excluded from analysis. Pairs of sequences were then identified, and the following parameters were calculated.

Abundance was calculated using the following formula:

$${Abundance}_{i , n} = \frac{{reads}_{i , n}}{{\sum }_{i}^{S} {reads}_{i , n}}$$

for a specific genotype i at timepoint n, and S represents the total number of unique genotypes at timepoint n after filtering as described above.

Fold-enrichment was calculated using the following formula:

$${Enrichment}_{i , n} = {log}_{2} \frac{{abundance}_{i , n}}{abundancei {,}_{0}}$$

for a specific genotype i at timepoint n, and abundance₀ represents the abundance after selection on agar plates as previously described before any liquid culture.

Post Facto Computational Modeling of Tether

For 3D modeling studies, we set up FARFAR2 simulations³⁴ using a crystal structure of the E. coli ribosome⁴⁰ (PDB code: 4YBB). Starting from that structure, we truncated the stemloops 23S rRNA Helix 101 (H101) and 16S rRNA helix 44 (h44), removing the residues that are deleted in all tethered ribosome constructs, and renumbered those residues to facilitate building a continuous RNA chain.

Using that initial structure as a template, we built the remaining residues of the tether using the FARFAR2 algorithm, conducted on 200 CPUs for 24 h, generating several thousand structures. We conducted simulations under two conditions: in one, only tether residues were resampled; in another, a junction on the 23S side of the tether was resampled as well.

All inputs and command files used in setting up computational modeling are available at github.com/everyday847/ribotv3_simulations.

Measurement of Orthogonal GFP Production

Combinations of potentially high-performant tether designs were identified from next generation sequencing results and built into a plasmid containing both an orthogonal tethered ribosome gene (oRibo-T) and an orthogonal superfolder GFP (o-sfGFP) coding sequence (mutated Shine-Dalgarno sequence)⁹. 10 ng of sequence-confirmed plasmids were then transformed into 25 μL of BL21(DE3) cells via electroporation, recovered in 1 mL of SOC, and plated on agar plates containing 100 μg/mL of carbenicillin. Individual colonies were picked (n=3) for inoculation of 100 μL of LB media containing 100 μg/mL carbenicillin. Cultures were incubated at 37° C. for 14-16 h with 2 mm continuous linear shaking in a plate reader (Agilent BioTek Synergy H1) and absorbance at 600 nm (OD600) was monitored to ensure saturation. After cultures reached saturation, each culture was diluted to an of ˜0.01 OD600 in fresh LB media containing 100 μg/mL of carbenicillin and 1 mM isopropyl β-D-1-thiogalactopyranoside (IPTG) to induce transcription of the orthogonal GFP gene. Cultured were incubated at 37° C. for 14-16 h with 2 mm continuous linear shaking in a plate reader and OD600 was monitored along with fluorescence (485/528 nm excitation/emission). Orthogonal GFP production (fluorescence) was normalized by OD600.

Growth Rate Characterization of Ribo-Tv3

A plasmid encoding tether sequences corresponding to Ribo-Tv3 (named pRTv3), was constructed using Gibson assembly31. 10 ng of pRTv3 was transformed into 50 μL of SQ171 cells 18 via electroporation and recovered in 500 μL of SOC at 37° C. for 2 h with shaking at 250 rpm.

After recovery, 1.5 mL of SOC was added and supplemented with 100 μg/mL carbenicillin and 0.25% (w/v) sucrose (final concentrations). After overnight (16-18 h) recovery at 37° C. with 250 rpm shaking, the cells were spun down (4000×g, 10 minutes) and plated on LB-agar plates containing 100 mg/m: carbenicillin, 5% sucrose, and 250 μg/mL erythromycin. Individual colonies were picked, and resistance to 100 μg/mL carbenicillin and sensitivity to 50 μg/mL kanamycin was checked on LB-agar plates to confirm successful swapping of ribosome plasmids in the SQ171 cells. Colonies that successfully replaced pCSacB32 with pRTv3 were carried through for analysis.

In a 96 well plate, 100 μL of LB media containing 100 μg/mL carbenicillin, 5% sucrose, and 250 μg/mL erythromycin was inoculated with a colony from an LB agar plate containing 100 μg/mL carbenicillin, 5% sucrose, and 250 μg/mL erythromycin and incubated for 14-16 h at 37° C. with 2 mm lateral shaking in a plate reader (Agilent BioTek Synergy H1). Absorbance at 600 nm was monitored to ensure cultures reached saturation. After incubation, cultures were diluted to A600 ˜0.05 (˜20-fold) in 100 μL of LB media containing 100 μg/mL carbenicillin, 5% sucrose, and 250 μg/mL erythromycin and incubated for 18 h at 37° C. with 2 mm lateral shaking, and absorbance at 600 nm (A600) was monitored.

Preparation of DECP-CME

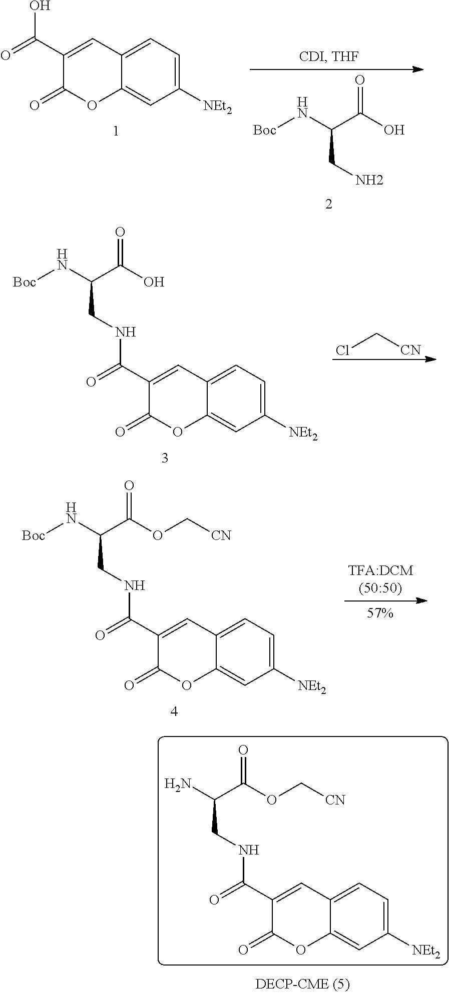

Cyanomethyl-2-amino-3-(7-(diethylamino)-2-oxo-2H-chromene-3-carboxamido) propanoate (DECP-CME, 5) was prepared with three steps using the synthetic methods previously described 36,41. First, 268 mg (1 mmol) of 7-(diethylamino)-2-oxo-2H-chromene-3-carboxylic acid (1) and 162 mg (1 mmol) of carbonyldiimidazole (CDI) were added to a flask and sealed with a septum. 5 mL of anhydrous DMF was added into the flask using an oven-dried syringe and stirred at room temperature for 2 h. 204 mg (1 mmol) of (R)-3-amino-2-((tert-butoxycarbonyl)amino)propanoic acid (2) was added and stirred overnight. The product was extracted with ethyl acetate after washing the crude reaction mixture with 1 M HCl, water, and brine. Second, 38 mL (0.6 mmol) of chloroacetonitrile and 104 mL (0.75 mmol) of triethylamine were added to 223 mg (0.5 mmol) of the purified 2-((tert-butoxycarbonyl)amino)-3-(7-(diethylamino)-2-oxo-2H-chromene-3-carboxamido) propanoic acid (3) in 1 mL of DCM and stirred overnight. The organic layer was washed with 1 M HCl, water, and brine and dried over MgSO4. 3) 1 mL of 50% of TFA solution in DCM was added to the purified cyanomethyl 2-((tert-butoxycarbonyl)amino)-3-(7-(diethylamino)-2-oxo-2H-chromene-3-carboxamido)propanoate (4) to deprotect the Boc group. The final product was dried under high vacuum and obtained as pale yellow powder (yield: 57%).

### Purification of RTv3 for In Vitro Translation Reactions

In brief, SQ171 cells harboring pRTv3 as the sole source of ribosomes were grown to mid-exponential phase (0.3-0.8 A600) in 500 mL of LB media containing 100 □g/mL carbenicillin and 250 □g/mL erythromycin. Cells were spun down, lysed using homogenization, and ribosomes were harvested using a sucrose cushion as described previously 25. Ribosome pellets were resuspended in Buffer C (10 mM pH 7.5 Tris Acetate, 60 mM ammonium chloride, 7.5 mM magnesium acetate, 0.5 mM ethylenediaminetetraacetic acid, and 2 mM dithiothreitol) and brought to a concentration of 15 mM (A260=625). Resuspended ribosomes were used directly in in vitro translation reactions.

### In Vitro Translation Reactions for Incorporation of DECP by RTv3

Preparation of DNA templates for RNAs. The DNA templates for flexizmyes and tRNAs preparation were synthesized as previously described 22,36. Sequences of the final DNA templated used for in vitro transcription by the T7 polymerase are:

| | |
|---|---|
| fMet (CAU) | 5′- |
| | GTAATACGACTCACTATAGGCGGGGTGGAGCAGCCTGGTAGCTCGTCGGGCTCATAA |
| | CCCGAAGATCGTCGGTTCAAATCCGGCCCCCGCAACCA-3′ (SEQ ID NO: 17) |
| eFx | 5′- |
| | GTAATACGACTCACTATAGGATCGAAAGATTTCCGCGGCCCCGAAAGGGGATTAGCG |
| | TTAGGT-3′ (SEQ ID NO: 18) |
| dFx | 5′- |
| | GTAATACGACTCACTATAGGATCGAAAGATTTCCGCATCCCCGAAAGGGTACATGGC |
| | GTTAGGT-3′ (SEQ ID NO: 19) |
| aFx | 5′- |
| | GTAATACGACTCACTATAGGATCGAAAGATTTCCGCACCCCCGAAAGGGGTAAGTGG |
| | CGTTAGGT-3′ (SEQ ID NO: 20) |

Preparation of Fxs and tRNAs.

Flexizymes (Fxs) and tRNAs were prepared using an in vitro transcription kit (HiScribe™ T7 High yield RNA synthesis kit, NEB E2040S) and purified by the previously reported methods 22.

Charging DECP into tRNA by Fx.

The acylation experiment was performed first using flexizyme with three flexizymes (e, d, and aFx). The Fx reaction was carried out as follows: 1 μL of 0.5 M HEPES (pH 7.5) or bicine (pH 8.8), 1 μL of 10 μM microhelix (mihx, tRNA mimic), and 3 μL of nuclease-free water were mixed in a PCR tube with 1 μL of 10 μM eFx, dFx, and aFx, respectively. The mixture was heated for 2 min at 95° C. and cooled down to room temperature over 5 min. 2 μL of 0.3 M MgCl2 in water was added to the mixture and incubated for 5 min at room temperature. Followed by the incubation of the reaction mixture on ice for 2 min, 2 μL of 25 mM DECP-CME in DMSO was then added to the reaction mixture. The reaction mixture was incubated for 16 h on ice in cold room. The optimal acylation reaction was determined by measuring the acylation yield using an acidic polyacrylamide gel (pH 5.2). tRNAfMet(AUG) was charged with DECP under the condition obtained from the mihx acylation experiment. The charged tRNA was precipitated using ethanol and used for in vitro translation without further purification.

In Vitro Protein Translation Reaction.

The non-canonical substrate incorporation experiment was performed using the PURExpress™ (Δribosome, Δaa, ΔtRNA, E3315Z) system. DECP-charged tRNAfMet(CAU) was dissolved in 1 μL of 1 mM NaOAc (pH 5.2) and added into 9 μL solution mixture containing 2 μL of Solution A, 1.2 μL of Factor mix, 1.8 μL of Ribo-T v3 (2.4 μM in final reaction), 1 μL of

endogenous tRNA mixture, 1 μL of DNA plasmid (130 ng μL-1), 1 μL of nuclease-free water, and

1 μL of 5 mM amino acid mixtures (Trp, Ser, His, Pro, Gln, Phe, Glu, Lys, and Thr). The reaction mixture was incubated in 37° C. for 2 h.

The target peptide produced in the PURE reaction was purified by using MagStrep (type 3) XT beads 5% suspension (IBA Lifesciences) which selectively pull down the target peptide bearing the Strep tag (WSHPQFEK) at the C-terminal region. After pulling down the target peptide, the magnetic beads were washed with the Strep-Tactin XT wash buffer (IBA Lifesciences) and treated with 0.1% SDS solution in water. The beads were heated at 95° C. in a PCR machine to denature the target peptide bound to the beads. The magnetic beads were removed on a magnet rack and the obtained peptide was analyzed by mass spectrometry.

DNA Primers Used in this Study.

Sequences are listed 5′ to 3′. For primers indicated with ‘\Phos\’, Phosphorylation performed on oligos with polynucleotide kinase (PNK) prior to PCR for use in blunt end ligation. ‘N’ indicates degenerate oligonucleotides. All oligonucleotides purchased from Integrated DNA Technologies (IDT).

| Use | Primer Name | Sequence (5′→3′) | Description |
|---|---|---|---|
| Broad | RTv3_BroadSample_ | AAGAAGTAGGTAGCTTAACCnnnnn | Forward primer used to install 5 |
| Sampling | 5N-f | TTGAGCTAACCGGTACTAATGAAC | degenerate nucleotides in Tl region, |
| Library | | C (SEQ ID NO: 21) | Broad Sampling Library |
| construction, | RTv3_BroadSample_ | AATCACAAAGTGGTAAGCGCCCTC | Reverse primer used to install 5 |
| insert | 5N-r | CnnnnnCTTACACACCCGGCCTATCA | degenerate nucleotides in T2 region, |
| | | A (SEQ ID NO: 22) | Broad Sampling Library |
| | RTv3_BroadSample_ | AAGAAGTAGGTAGCTTAACCnnnnn | Forward primer used to install 6 |
| | 6N-f | nTTGAGCTAACCGGTACTAATGAA | degenerate nucleotides inTl region, |
| | | CC (SEQ ID NO: 23) | Broad Sampling Library |
| | RTv3_BroadSample_ | AATCACAAAGTGGTAAGCGCCCTC | Reverse primer used to install 6 |
| | 6N-r | CnnnnnnCTTACACACCCGGCCTATC | degenerate nucleotides in T2 region, |
| | | AA (SEQ ID NO: 24) | Broad Sampling Library |
| | RTv3_BroadSample_ | AAGAAGTAGGTAGCTTAACCnnnnn | Forward primer used to install 7 |
| | 7N-f | nTTGAGCTAACCGGTACTAATGAA | degenerate nucleotides inTl region, |
| | | CC (SEQ ID NO: 25) | Broad Sampling Library |
| | RTv3_BroadSample_ | AATCACAAAGTGGTAAGCGCCCTC | Reverse primer used to install 7 |
| | 7N-r | CnnnnnnnCTTACACACCCGGCCTAT | degenerate nucleotides in T2 region, |
| | | CAA (SEQ ID NO: 26) | Broad Sampling Library |
| | RTv3_BroadSample_ | AAGAAGTAGGTAGCTTAACCnnnnn | Forward primer used to install 8 |
| | 8N-f | nnnTTGAGCTAACCGGTACTAATGA | degenerate nucleotides inTl region, |
| | | ACC (SEQ ID NO: 27) | Broad Sampling Library |
| | RTv3_BroadSample_ | AATCACAAAGTGGTAAGCGCCCTC | Reverse primer used to install 8 |
| | 8N-r | CnnnnnnnCTTACACACCCGGCCTAT | degenerate nucleotides in T2 region, |
| | | CAA (SEQ ID NO: 28) | Broad Sampling Library |
| | RTv3_BroadSample_ | AAGAAGTAGGTAGCTTAACCnnnnn | Forward primer used to install 9 |
| | 9N-f | nnnnTTGAGCTAACCGGTACTAATG | degenerate nucleotides inTl region, |
| | | AACC (SEQ ID NO: 29) | Broad Sampling Library |
| | RTv3_BroadSample_ | AATCACAAAGTGGTAAGCGCCCTC | Reverse primer used to install 9 |
| | 9N-r | CnnnnnnnnnCTTACACACCCGGCCTA | degenerate nucleotides in T2 region, |
| | | TCAA (SEQ ID NO: 30) | Broad Sampling Library |
| | RTv3_BroadSample_ | AAGAAGTAGGTAGCTTAACCnnnnn | Forward primer used to install 10 |
| | 10N-f | nnnnTTGAGCTAACCGGTACTAATG | degenerate nucleotides inTl region, |
| | | AACC (SEQIDNO: 31) | Broad Sampling Library |
| | RTv3_BroadSample_ | AATCACAAAGTGGTAAGCGCCCTC | Reverse primer used to install 10 |
| | 10N-r | CnnnnnnnnnnCTTACACACCCGGCCT | degenerate nucleotides in T2 region, |
| | | ATCAA (SEQ ID NO: 32) | Broad Sampling Library |
| | RTv3_BroadSample_ | AAGAAGTAGGTAGCTTAACCnnnnn | Forward primer used to install 11 |
| | 11N-f | nnnnnnTTGAGCTAACCGGTACTAAT | degenerate nucleotides inTl region, |
| | | GAACC (SEQIDNO: 33) | Broad Sampling Library |
| | RTv3_BroadSample_ | AATCACAAAGTGGTAAGCGCCCTC | Reverse primer used to install 11 |
| | 11N-r | CnnnnnnnnnnnCTTACACACCCGGCC | degenerate nucleotides in T2 region, |
| | | TATCAA (SEQ ID NO: 34) | Broad Sampling Library |
| | RTv3_BroadSample_ | AAGAAGTAGGTAGCTTAACCnnnnn | Forward primer used to install 12 |
| | 12N-f | nnnnnnnTTGAGCTAACCGGTACTAA | degenerate nucleotides inTl region, |
| | | TGAACC (SEQ ID NO: 35) | Broad Sampling Library |
| | RTv3_BroadSample_ | AATCACAAAGTGGTAAGCGCCCTC | Reverse primer used to install 12 |
| | 12N-r | CnnnnnnnnnnnnCTTACACACCCGGC | degenerate nucleotides in T2 region, |
| | | CTATCAA (SEQ ID NO: 36) | Broad Sampling Library |
| | RTv3_BroadSample_ | AAGAAGTAGGTAGCTTAACCnnnnn | Forward primer used to install 13 |
| | 13N-f | nnnnnnnnTTGAGCTAACCGGTACTA | degenerate nucleotides inTl region, |
| | | ATGAACC (SEQ ID NO: 37) | Broad Sampling Library |
| | RTv3_BroadSample_ | AATCACAAAGTGGTAAGCGCCCTC | Reverse primer used to install 13 |
| | 13N-r | CnnnnnnnnnnnnnCTTACACACCCGG | degenerate nucleotides in T2 region, |
| | | CCTATCAA (SEQ ID NO: 38) | Broad Sampling Library |
| | RTv3_BroadSample_ | AAGAAGTAGGTAGCTTAACCnnnnn | Forward primer used to install 14 |
| | 14N-f | nnnnnnnnTTGAGCTAACCGGTACTA | degenerate nucleotides inTl region, |
| | | ATGAACC (SEQ ID NO: 39) | Broad Sampling Library |
| | RTv3_BroadSample_ | AATCACAAAGTGGTAAGCGCCCTC | Reverse primer used to install 14 |
| | 14N-r | CnnnnnnnnnnnnnCTTACACACCCGG | degenerate nucleotides in T2 region, |
| | | CCTATCAA (SEQ ID NO: 40) | Broad Sampling Library |
| | RTv3_BroadSample_ | AAGAAGTAGGTAGCTTAACCnnnnn | Forward primer used to install 15 |
| | 15N-f | nnnnnnnnnnTTGAGCTAACCGGTACT | degenerate nucleotides inTl region, |
| | | AATGAACC (SEQ ID NO: 41) | Broad Sampling Library |
| | RTv3_BroadSample_ | AATCACAAAGTGGTAAGCGCCCTC | Reverse primer used to install 15 |
| | 15N-r | CnnnnnnnnnnnnnCTTACACACCCGG | degenerate nucleotides in T2 region, |
| | | CCTATCAA (SEQ ID NO: 42) | Broad Sampling Library |
| Tether-H101 | d1_RTv2-f | GAAGTAGGTAGCTTAACCcaatgaacaa | Forward primer for 1 residue |
| Junction | | ttggaGCGTTGAGCTAACCGGTACTA | deletion in Tether-H101 junction |
| Library | | ATGAAC (SEQ ID NO: 43) | |
| construction, | d1_RTv2-r | AATCACAAAGTGGTAAGCGCCCTC | Reverse primer for 1 residue |
| insert | | CactagttatcGCGCTTACACACCCGGC | deletion in Tether-H10 junction |
| | | CTATCAA (SEQ ID NO: 44) | |
| | d2_RTv2-f | GAAGTAGGTAGCTTAACCcaatgaacaa | Forward primer for 2 residue |
| | | ttggaCGTTGAGCTAACCGGTACTAA | deletion in Tether-H101 junction |
| | | TGAACC (SEQ ID NO: 45) | |
| | d2_RTv2-r | AATCACAAAGTGGTAAGCGCCCTC | Reverse primer for 2 residue |
| | | CactagttatcCGCTTACACACCCGGCC | deletion in Tether-H101 junction |
| | | TATCAA (SEQ ID NO: 46) | |
| | d3_RTv2-f | GAAGTAGGTAGCTTAACCcaatgaacaa | Forward primer for 3 residue |
| | | ttggaGTTGAGCTAACCGGTACTAAT | deletion in Tether-H101 junction |
| | | GAACC (SEQ ID NO: 47) | |
| | d3_RTv2-f | AATCACAAAGTGGTAAGCGCCCTC | Reverse primer for 3 residue |
| | | CactagttatcGCTTACACACCCGGCCT | deletion in Tether-H101 junction |
| | | ATCAA (SEQ ID NO: 48) | |
| | d4_RTv2-f | AAGAAGTAGGTAGCTTAACCcaatga | Forward primer for 4 residue |
| | | acaattggaTTGAGCTAACCGGTACTAA | deletion in Tether-H101 junction |
| | | TGAACC (SEQ ID NO: 49) | |
| | d4_RTv2-r | AATCACAAAGTGGTAAGCGCCCTC | Reverse primer for 4 residue |
| | | CactagttatcCTTACACACCCGGCCTA | deletion in Tether-H101 junction |
| | | TCA (SEQ ID NO: 50) | |
| | d5_RTv2-f | AAGAAGTAGGTAGCTTAACCcaatga | Forward primer for 5 residue |
| | | acaattggaTGAGCTAACCGGTACTAAT | deletion in Tether-H101 junction |
| | | GAACC (SEQ ID NO: 51) | |
| | d5_RTv2-r | AATCACAAAGTGGTAAGCGCCCTC | Reverse primer for 5 residue |
| | | CactagttatcTTACACACCCGGCCTAT | deletion in Tether-H101 junction |
| | | CAA (SEQ ID NO: 52) | |
| Designed | RTv3_ | AATCACAAAGTGGTAAGCGCCCTC | Forward primer for 5 degenerate |
| Junction | DesignedJunc_ | CnnnnnGCCTTACACACCCGGCCTAT | residues for Designed Junction |
| Library | 5N-r | CAA (SEQ ID NO: 53) | Library |
| construction, | RTv3_ | AATCACAAAGTGGTAAGCGCCCTC | Reverse primer for 5 degenerate |
| insert | DesignedJunc_ | CnnnnnGTCCTTACACACCCGGCCTA | residues for Designed Junction |
| | 5N-r | TCAA (SEQ ID NO: 54) | Library |
| | RTv3_ | AAGAAGTAGGTAGCTTAACCnnnnn | Forward primer for 5 degenerate |
| | DesignedJunc_ | nGCTTGAGCTAACCGGTACTAATG | residues for Designed Junction |
| | 6N-f | AACC (SEQ ID NO: 55) | Library |
| | RTv3_ | AATCACAAAGTGGTAAGCGCCCTC | Reverse primer for 5 degenerate |
| | DesignedJunc_ | CnnnnnnGCCTTACACACCCGGCCTA | residues for Designed Junction |
| | 6N-r | TCAA (SEQ ID NO: 56) | Library |
| | RTv3_ | AAGAAGTAGGTAGCTTAACCnnnnn | Forward primer for 5 degenerate |
| | DesignedJunc_ | nnGCTTGAGCTAACCGGTACTAATG | residues for Designed Junction |
| | 7N-f | AACC (SEQ ID NO: 57) | Library |
| | RTv3_ | AATCACAAAGTGGTAAGCGCCCTC | Reverse primer for 5 degenerate |
| | DesignedJunc_ | CnnnnnnnGCCTTACACACCCGGCCT | residues for Designed Junction |
| | 7N-r | ATCAA (SEQ ID NO: 58) | Library |
| | RTv3_ | AAGAAGTAGGTAGCTTAACCnnnnn | Forward primer for 5 degenerate |
| | DesignedJunc_ | nnnGCTTGAGCTAACCGGTACTAAT | residues for Designed Junction |
| | 8N-f | GAACC (SEQ ID NO: 59) | Library |
| | RTv3_ | AATCACAAAGTGGTAAGCGCCCTC | Reverse primer for 5 degenerate |
| | DesignedJunc_ | CnnnnnnnnGCCTTACACACCCGGCC | residues for Designed Junction |
| | 8N-r | TATCAA (SEQ ID NO: 60) | Library |
| | RTv3_ | AAGAAGTAGGTAGCTTAACCnnnnn | Forward primer for 5 degenerate |
| | DesignedJunc_ | nnnnGCTTGAGCTAACCGGTACTAA | residues for Designed Junction |
| | 9N-f | TGAACC (SEQ ID NO: 61) | Library |
| | RTv3_ | AATCACAAAGTGGTAAGCGCCCTC | Reverse primer for 5 degenerate |
| | DesignedJunc_ | CnnnnnnnnGCCTTACACACCCGGCC | residues for Designed Junction |
| | N-r | TATCAA (SEQ ID NO: 62) | Library |
| Backbone | Ribo-T_lib_bb-f | GGAGGGCGCTTACCACTTTGTGAT | Forward primer for amplification of |
| for Ribo-T v3 | | T (SEQ ID NO: 63) | backbone with library inserts, |
| library | | | assemble by isothermal assembly |
| construction | Ribo-T_lib_bb-r | GGTTAAGCTACCTACTTCTTTTGCA | Reverse primer for amplification of |
| amplification | | (SEQ ID NO: 64) | backbone with library inserts, |
| | | | assemble by isothermal assembly |
| Testing PCR1 | T1-T2_PCR1_GA_-f | GGAACGTTGAAGACGACGACGTTG | Forward primer for PCR1, compatible |
| compati- | | ATAGG (SEQ ID NO: 65) | for isothermal assembly |
| bility with | T1-T2_PCR1_GA-r | CCTATCAACGTCGTCGTCTTCAACG | Reverse primer for PCR1, compatible |
| different | | TTCCACGGTTCATTAGTACCGGTTA | for isothermal assembly |
| ligation | | GC (SEQ ID NO: 66) | |
| methods | \Phos\T1- | GGAACGTTGAAGACGACGACGTTG | Forward primer for PCR1, compatible |
| | T2_PCR1_blunt-f | ATAGG (SEQ ID NO: 67) | for blunt end ligation |
| | | | (phosphorylated prior to PCR) |
| | \Phos\T1- | CACGGTTCATTAGTACCGGTTAGC | Reverse primer for PCR1, compatible |
| | T2_PCR1_blunt-r | (SEQ ID NO: 68) | for blunt end ligation |
| | | | (phosphorylated prior to PCR) |
| | T1- | agatggatccGGAACGTTGAAGACGAC | Forward primer for PCR1, compatible |
| | T2 PCR1_BamHI- | GACGTTGATAGG (SEQ ID NO: 69) | for digestion with BamHI prior to |
| | for | | ligation |
| | T1- | ggatccatctCACGGTTCATTAGTACCG | Reverse primer for PCR1, compatible |
| | T2_PCR1_BamHI- | GTTAGC (SEQ ID NO: 70) | for digestion with BamHI prior to |
| | rev | | ligation |
| | T1-T2_PCR1_SapI- | gctcttcagcgGGAACGTTGAAGACGAC | Forward primer for PCR1, compatible |
| | for | GACGTTGATAGG (SEQ ID NO: 71) | for digestion with SapI prior to |
| | | | ligation |
| | T1-T2_PCR1_SapI- | ggctcttcacgcCACGGTTCATTAGTACC | Reverse primer for PCR1, compatible |
| | rev | GGTTAGC (SEQ ID NO: 72) | for digestion with SapI prior to |
| | | | ligation |
| PCR2 | T1-T2-PCR2-for | AGTGGGTTGCAAAAGAAGTAGGTA | Forward primer for PCR2 |
| | | GC (SEQ ID NO: 73) | |
| | T1-T2-PCR2-rev | CCAGTCATGAATCACAAAGTGGTA | Reverse primer for PCR2 |
| | | AGC (SEQ ID NO: 74) | |

Comparisons of orthogonal sfGFP production by multiple Ribo-T v3 candidates (FIG. 5C) compared to Ribo-T v2. One-sided Welch's t-test performed to compare Ribo-T v3 candidates to Ribo-T v2. T1 and T2 sequences are shown 5′ to 3′. Data shown representative of three independent experiments, and within each experiment, data from three replicates per T1 and T2 sequence genotype used to calculate standard deviation and perform t-test. Experiment and analysis were performed to analyze which Ribo-T v3 candidates had greater orthogonal sfGFP synthesis ability. * marks sequence chosen as Ribo-T v3.

| | | Normalized sfGFP | | |
|---|---|---|---|---|
| | | expression | Standard | P- |
| T1 sequence | T2 sequence | (fluorescence/A₆₀₀) | Deviation | value |
| GUUAUA | AUCCCAGG | 13457 | 103 | 0.000145 |
| GUUAUA | UCACAAC | 15196 | 871 | 0.000362 |
| GUUAUA | GACCUUCG | 12628 | 733 | 0.002386 |
| GUUAUA | ACAUAAUG | 6998 | 233 | 0.000793 |
| AGUCAAUAA | AUCCCAGG | 12997 | 222 | 0.000300 |
| AGUCAAUAA | UCACAAC | 13597 | 834 | 0.001172 |
| AGUCAAUAA* | GACCUUCG* | 15097 | 682 | 0.000207 |
| AGUCAAUAA | ACAUAAUG | 12327 | 543 | 0.001885 |
| CAUCAUGG | AUCCCAGG | 10482 | 525 | 0.061960 |
| CAUCAUGG | UCACAAC | 12729 | 1559 | 0.015705 |
| CAUCAUGG | GACCUUCG | 10866 | 1221 | 0.092446 |
| CAUCAUGG | ACAUAAUG | 13455 | 979 | 0.002063 |
| AUAUAAU | AUCCCAGG | 14094 | 483 | 0.000227 |
| AUAUAAU | UCACAAC | 13501 | 1135 | 0.003005 |
| AUAUAAU | GACCUUCG | 13572 | 1896 | 0.012928 |
| AUAUAAU | ACAUAAUG | 6057 | 428 | 0.000444 |
| RTv2 | RTv2 | 9629 | 550 | 1 |
| (CAAUGAACAAUUGGA) | (GAUAACUAGU) | | | |
| (SEQ ID NO: 75) | | | | |
| WT (no tether) | WT (no tether) | 673 | 44 | — |

Code Availability

All inputs and command files used in setting up computational modeling are available at github.com/everyday847/ribotv3_simulations.

### REFERENCES

1 Dedkova, L. M., Fahmi, N. E., Golovine, S. Y. & Hecht, S. M. Enhanced D-amino acid incorporation into protein by modified ribosomes. Journal of the American Chemical Society 125, 6616-6617 (2003).
2 Dedkova, L. M., Fahmi, N. E., Golovine, S. Y. & Hecht, S. M. Construction of modified ribosomes for incorporation of D-amino acids into proteins. Biochemistry 45, 15541-15551 (2006).
3 Dedkova, L. M. et al. β-Puromycin selection of modified ribosomes for in vitro incorporation of β-amino acids. Biochemistry 51, 401-415 (2012).
4 Dedkova, L. M. & Hecht, S. M. Expanding the scope of protein synthesis using modified ribosomes. Journal of the American Chemical Society 141, 6430-6447 (2019).
5 Des Soye, B. J., Patel, J. R., Isaacs, F. J. & Jewett, M. C. Repurposing the translation apparatus for synthetic biology. Current opinion in chemical biology 28, 83-90 (2015).
6 Ellefson, J. W. et al. Synthetic evolutionary origin of a proofreading reverse transcriptase. Science 352, 1590-1593 (2016).
7 Hammerling, M. J., Fritz, B. R., Yoesep, D. J., Carlson, E. D. & Jewett, M. C. In vitro ribosome synthesis and evolution through ribosome display. Nature communications 11, 1-10 (2020).
8 Maini, R. et al. Protein synthesis with ribosomes selected for the incorporation of j-amino acids. Biochemistry 54, 3694-3706 (2015).
9 Carlson, E. D. et al. Engineered ribosomes with tethered subunits for expanding biological function. Nature communications 10, 1-13 (2019).
10 Sailer, Z. R. & Harms, M. J. Molecular ensembles make evolution unpredictable. Proceedings of the National Academy of Sciences 114, 11938-11943 (2017).
11 Romero, P. A. & Arnold, F. H. Exploring protein fitness landscapes by directed evolution. Nature reviews Molecular cell biology 10, 866-876 (2009).
12 Sarkisyan, K. S. et al. Local fitness landscape of the green fluorescent protein. Nature 533, 397-401 (2016).
13 Ramakrishnan, V. Ribosome structure and the mechanism of translation. Cell 108, 557-572 (2002).
14 Schmied, W. H. et al. Controlling orthogonal ribosome subunit interactions enables evolution of new function. Nature 564, 444-448 (2018).
15 Fried, S. D., Schmied, W. H., Uttamapinant, C. & Chin, J. W. Ribosome subunit stapling for orthogonal translation in E. coli. Angewandte Chemie 127, 12982-12985 (2015).
16 Liu, C. C., Jewett, M. C., Chin, J. W. & Voigt, C. A. Toward an orthogonal central dogma. Nature chemical biology 14, 103-106 (2018).
17 Liu, Y., Kim, D. S. & Jewett, M. C. Repurposing ribosomes for synthetic biology. Current opinion in chemical biology 40, 87-94 (2017).
18 Orelle, C. et al. Protein synthesis by ribosomes with tethered subunits. Nature 524, 119-124 (2015).
19 Rackham, O. & Chin, J. W. A network of orthogonal ribosome mRNA pairs. Nature chemical biology 1, 159-166 (2005).
20 Neumann, H., Wang, K., Davis, L., Garcia-Alai, M. & Chin, J. W. Encoding multiple unnatural amino acids via evolution of a quadruplet-decoding ribosome. Nature 464, 441-444 (2010).
21 Wang, K., Neumann, H., Peak-Chew, S. Y. & Chin, J. W. Evolved orthogonal ribosomes enhance the efficiency of synthetic genetic code expansion. Nature biotechnology 25, 770-777 (2007).
22 Goto, Y., Katoh, T. & Suga, H. Flexizymes for genetic code reprogramming. Nature protocols 6, 779-790 (2011).
23 Melo Czekster, C., Robertson, W. E., Walker, A. S., Söll, D. & Schepartz, A. In vivo biosynthesis of a β-amino acid-containing protein. Journal of the American Chemical Society 138, 5194-5197 (2016).
   24 Chin, J. W. Expanding and reprogramming the genetic code. Nature 550, 53-60 (2017).
25 Jewett, M. C., Fritz, B. R., Timmerman, L. E. & Church, G. M. In vitro integration of ribosomal RNA synthesis, ribosome assembly, and translation. Molecular systems biology 9, 678 (2013).
26 Hui, A. & de Boer, H. A. Specialized ribosome system: preferential translation of a single mRNA species by a subpopulation of mutated ribosomes in Escherichia coli. Proc Natl Acad Sci USA 84, 4762-4766, doi:10.1073/pnas.84.14.4762 (1987).
27 Rackham, O. & Chin, J. W. Cellular logic with orthogonal ribosomes. Journal of the American Chemical Society 127, 17584-17585 (2005).
28 Cho, N. et al. De novo assembly and next-generation sequencing to analyse full-length gene variants from codon-barcoded libraries. Nature communications 6, 1-9 (2015).
29 Yoo, J. I., Daugherty, P. S. & O'Malley, M. A. Bridging non-overlapping reads illuminates high-order epistasis between distal protein sites in a GPCR. Nature communications 11, 1-12 (2020).
30 Borgström, E. et al. Phasing of single DNA molecules by massively parallel barcoding. Nature communications 6, 7173 (2015).
31 Gibson, D. G. et al. Enzymatic assembly of DNA molecules up to several hundred kilobases. Nature methods 6, 343-345 (2009).
32 Carlson, E. D. et al. Engineered ribosomes with tethered subunits for expanding biological function. Nature communications 10, 1-13 (2019).
33 Asai, T., Zaporojets, D., Squires, C. & Squires, C. L. An Escherichia coli strain with all chromosomal rRNA operons inactivated: complete exchange of rRNA genes between bacteria. Proceedings of the National Academy of Sciences 96, 1971-1976 (1999).
34 Lorenz, R. et al. ViennaRNA Package 2.0. Algorithms for molecular biology 6, 26 (2011).
35 Watkins, A. M., Rangan, R. & Das, R. FARFAR2: Improved de novo Rosetta prediction of complex global RNA folds. Structure (2020).
36 Noeske, J. et al. High-resolution structure of the Escherichia coli ribosome. Nature structural & molecular biology 22, 336-341 (2015).
37 Lee, J., Schwarz, K. J., Kim, D. S., Moore, J. S. & Jewett, M. C. Ribosome-mediated polymerization of long chain carbon and cyclic amino acids into peptides in vitro. Nature Communications 11, 4304, doi:10.1038/s41467-020-18001-x (2020).
38 Lee, J. et al. Expanding the limits of the second genetic code with ribozymes. Nature communications 10, 1-12 (2019).
39 Lee, J. et al. Ribosomal incorporation of cyclic β-amino acids into peptides using in vitro translation. Chemical Communications 56, 5597-5600 (2020).
40 Aleksashin, N. A. et al. A fully orthogonal system for protein synthesis in bacterial cells. Nature communications 11, 1-11 (2020).

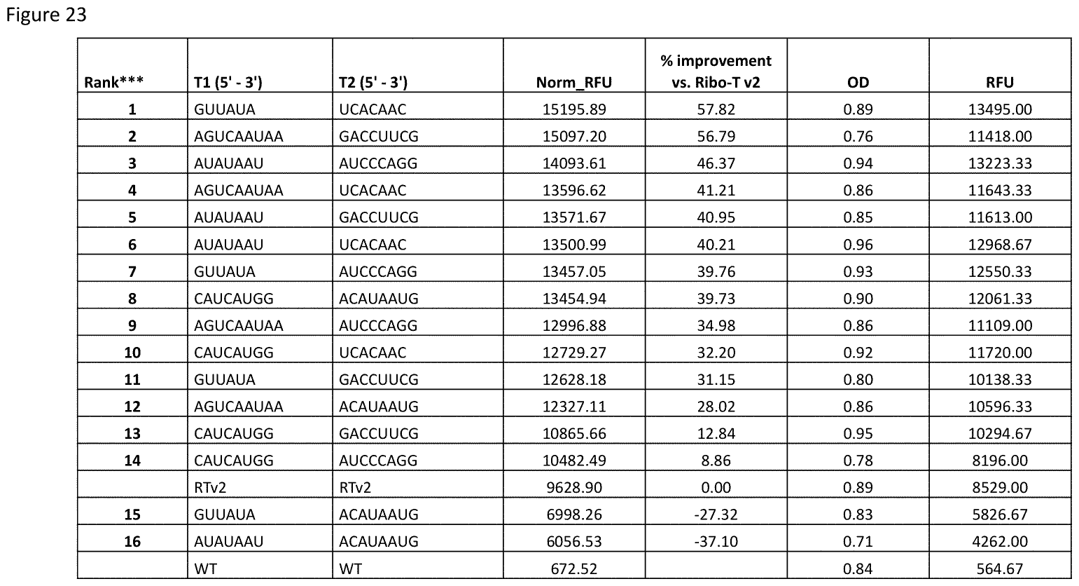

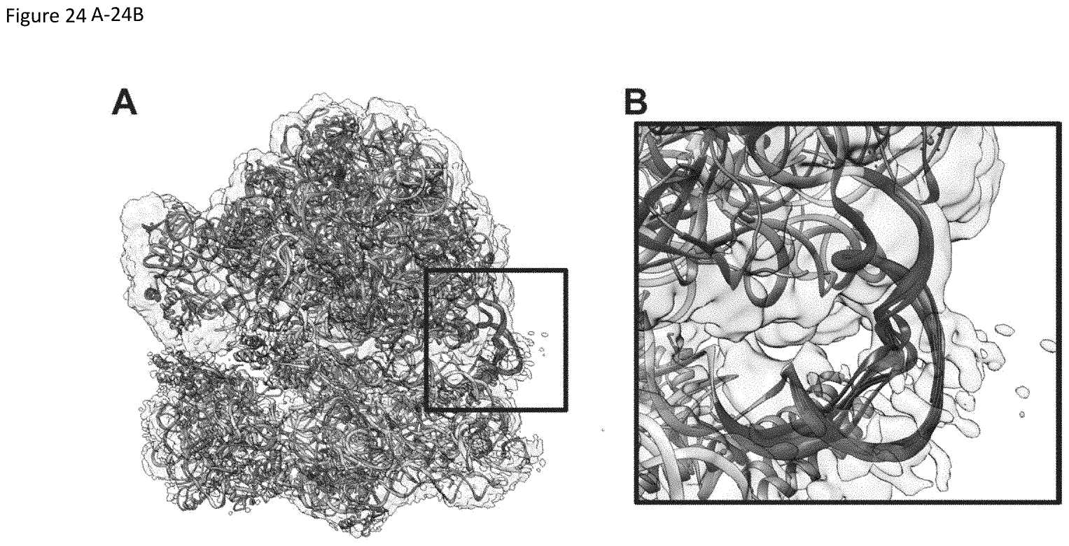

Full Sequences of modified ribosome RNA including tether pairs 1-16 of FIG. 23 are provided below. Disclosed herein are engineered ribosomes comprising the full sequences of any one of Pair 1-16 full sequence, as shown below.

Pair 1: Full Sequence [SEQ ID NO: 1]
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
GUUAUAGUCUUGAGCUAACCGGUACUAAUGAACCGUGAGGCUUAACCGAGAGGUUAAGCGACU
AAGCGUACACGGUGGAUGCCCUGGCAGUCAGAGGCGAUGAAGGACGUGCUAAUCUGCGAUAAG
CGUCGGUAAGGUGAUAUGAACCGUUAUAACCGGCGAUUUCCGAAUGGGGAAACCCAGUGUGUU
UCGACACACUAUCAUUAACUGAAUCCAUAGGUUAAUGAGGCGAACCGGGGGAACUGAAACAUC
UAAGUACCCCGAGGAAAAGAAAUCAACCGAGAUUCCCCCAGUAGCGGCGAGCGAACGGGGAGC
AGCCCAGAGCCUGAAUCAGUGUGUGUGUUAGUGGAAGCGUCUGGAAAGGCGCGCGAUACAGGG
UGACAGCCCCGUACACAAAAAUGCACAUGCUGUGAGCUCGAUGAGUAGGGCGGGACACGUGGU
AUCCUGUCUGAAUAUGGGGGGACCAUCCUCCAAGGCUAAAUACUCCUGACUGACCGAUAGUGA
ACCAGUACCGUGAGGGAAAGGCGAAAAGAACCCCGGCGAGGGGAGUGAAAAAGAACCUGAAAC
CGUGUACGUACAAGCAGUGGGAGCACGCUUAGGCGUGUGACUGCGUACCUUUUGUAUAAUGGG
UCAGCGACUUAUAUUCUGUAGCAAGGUUAACCGAAUAGGGGAGCCGAAGGGAAACCGAGUCUU
AACUGGGCGUUAAGUUGCAGGGUAUAGACCCGAAACCCGGUGAUCUAGCCAUGGGCAGGUUGA
AGGUUGGGUAACACUAACUGGAGGACCGAACCGACUAAUGUUGAAAAAUUAGCGGAUGACUUG
UGGCUGGGGGUGAAAGGCCAAUCAAACCGGGAGAUAGCUGGUUCUCCCCGAAAGCUAUUUAGG
UAGCGCCUCGUGAAUUCAUCUCCGGGGGUAGAGCACUGUUUCGGCAAGGGGGUCAUCCCGACU
UACCAACCCGAUGCAAACUGCGAAUACCGGAGAAUGUUAUCACGGGAGACACACGGCGGGUGC
UAACGUCCGUCGUGAAGAGGGAAACAACCCAGACCGCCAGCUAAGGUCCCAAAGUCAUGGUUA
AGUGGGAAACGAUGUGGGAAGGCCCAGACAGCCAGGAUGUUGGCUUAGAAGCAGCCAUCAUUU
AAAGAAAGCGUAAUAGCUCACUGGUCGAGUCGGCCUGCGCGGAAGAUGUAACGGGGCUAAACC
AUGCACCGAAGCUGCGGCAGCGACGCUUAUGCGUUGUUGGGUAGGGGAGCGUUCUGUAAGCCU
GCGAAGGUGUGCUGUGAGGCAUGCUGGAGGUAUCAGAAGUGCGAAUGCUGACAUAAGUAACGA
UAAAGCGGGUGAAAAGCCCGCUCGCCGGAAGACCAAGGGUUCCUGUCCAACGUUAAUCGGGGC
AGGGUGAGUCGACCCCUAAGGCGAGGCCGAAAGGCGUAGUCGAUGGGAAACAGGUUAAUAUUC
CUGUACUUGGUGUUACUGCGAAGGGGGGACGGAGAAGGCUAUGUUGGCCGGGCGACGGUUGUC
CCGGUUUAAGCGUGUAGGCUGGUUUUCCAGGCAAAUCCGGAAAAUCAAGGCUGAGGCGUGAUG
ACGAGGCACUACGGUGCUGAAGCAACAAAUGCCCUGCUUCCAGGAAAAGCCUCUAAGCAUCAG
GUAACAUCAAAUCGUACCCCAAACCGACACAGGUGGUCAGGUAGAGAAUACCAAGGCGCUUGA
GAGAACUCGGGUGAAGGAACUAGGCAAAAUGGUGCCGUAACUUCGGGAGAAGGCACGCUGAUA
UGUAGGUGAGGUCCCUCGCGGAUGGAGCUGAAAUCAGUCGAAGAUACCAGCUGGCUGCAACUG
UUUAUUAAAAACACAGCACUGUGCAAACACGAAAGUGGACGUAUACGGUGUGACGCCUGCCCG
GUGCCGGAAGGUUAAUUGAUGGGGUUAGCGCAAGCGAAGCUCUUGAUCGAAGCCCCGGUAAAC
GGCGGCCGUAACUAUAACGGUCCUAAGGUAGCGAAAUUCCUUGUCGGGUAAGUUCCGACCUGC
ACGAAUGGCGUAAUGAUGGCCAGGCUGUCUCCACCCGAGACUCAGUGAAAUUGAACUCGCUGU
GAAGAUGCAGUGUACCCGCGGCAAGACGGAAAGACCCCGUGAACCUUUACUAUAGCUUGACAC
UGAACAUUGAGCCUUGAUGUGUAGGAUAGGUGGGAGGCUUUGAAGUGUGGACGCCAGUCUGCA
UGGAGCCGAGCUUGAAAUACCACCCUUUAAUGUUUGAUGUUCUAACGUUGACCGGUAAUCCGG
GUUGCGGACAGUGUCUGGUGGGUAGUUUGACUGGGGCGGUCUCCUCCUAAAGAGUAACGGAGG
AGCACGAAGGUUGGCUAAUCCUGGUCGGACAUCAGGAGGUUAGUGCAAUGGCAUAAGCCAGCU
UGACUGCGAGCGUGACGGCGCGAGCAGGUGCGAAAGCAGGUCAUAGUGAUCCGGUGGUUCUGA
AUGGAAGGGCCAUCGCUCAACGGAUAAAAGGUACUCCGGGGAUAACAGGCUGAUACCGCCCAA
GAGUUCAUAUCGACGGCGGUGUUUGGCACCUCGAUGUCGGCUCAUCACAUCCUGGGGCUGAAG
UAGGUCCCAAGGGUAUGGCUGUUCGCCAUUUAAAGUGGUACGCGAGCUGGGUUUAGAACGUCG
UGAGACAGUUCGGUCCCUAUCUGCCGUGGGCGCUGGAGAACUGAGGGGGGCUGCUCCUAGUAC
GAGAGGACCGGAGUGGACGCAUCACUGGUGUUCGGGUUGUCAUGCCAAUGGCACUGCCCGGUA
GCUAAAUGCGGAAGAGAUAAGUGCUGAAAGCAUCUAAGCACGAAACUUGCCCCGAGAUGAGUU
CUCCCUGACCCUUUAAGGGUCCUGAAGGAACGUUGAAGACGACGACGUUGAUAGGCCGGGUGU
GUAAGGACUCACAACGGAGGGCGCUUACCACUUUGUGAUUCAUGACUGGGGUGAAGUCGUAAC
AAGGUAACCGUAGGGGAACCUGCGGUUGGAUCACUGUGGUA
Pair 2: Full Sequence (SEQ ID NO: 2)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
AGUCAAUAAGUCUUGAGCUAACCGGUACUAAUGAACCGUGAGGCUUAACCGAGAGGUUAAGCG
ACUAAGCGUACACGGUGGAUGCCCUGGCAGUCAGAGGCGAUGAAGGACGUGCUAAUCUGCGAU
AAGCGUCGGUAAGGUGAUAUGAACCGUUAUAACCGGCGAUUUCCGAAUGGGGAAACCCAGUGU
GUUUCGACACACUAUCAUUAACUGAAUCCAUAGGUUAAUGAGGCGAACCGGGGGAACUGAAAC
AUCUAAGUACCCCGAGGAAAAGAAAUCAACCGAGAUUCCCCCAGUAGCGGCGAGCGAACGGGG
AGCAGCCCAGAGCCUGAAUCAGUGUGUGUGUUAGUGGAAGCGUCUGGAAAGGCGCGCGAUACA
GGGUGACAGCCCCGUACACAAAAAUGCACAUGCUGUGAGCUCGAUGAGUAGGGCGGGACACGU
GGUAUCCUGUCUGAAUAUGGGGGGACCAUCCUCCAAGGCUAAAUACUCCUGACUGACCGAUAG
UGAACCAGUACCGUGAGGGAAAGGCGAAAAGAACCCCGGCGAGGGGAGUGAAAAAGAACCUGA
AACCGUGUACGUACAAGCAGUGGGAGCACGCUUAGGCGUGUGACUGCGUACCUUUUGUAUAAU
GGGUCAGCGACUUAUAUUCUGUAGCAAGGUUAACCGAAUAGGGGAGCCGAAGGGAAACCGAGU
CUUAACUGGGCGUUAAGUUGCAGGGUAUAGACCCGAAACCCGGUGAUCUAGCCAUGGGCAGGU
UGAAGGUUGGGUAACACUAACUGGAGGACCGAACCGACUAAUGUUGAAAAAUUAGCGGAUGAC
UUGUGGCUGGGGGUGAAAGGCCAAUCAAACCGGGAGAUAGCUGGUUCUCCCCGAAAGCUAUUU
AGGUAGCGCCUCGUGAAUUCAUCUCCGGGGGUAGAGCACUGUUUCGGCAAGGGGGUCAUCCCG
ACUUACCAACCCGAUGCAAACUGCGAAUACCGGAGAAUGUUAUCACGGGAGACACACGGCGGG
UGCUAACGUCCGUCGUGAAGAGGGAAACAACCCAGACCGCCAGCUAAGGUCCCAAAGUCAUGG
UUAAGUGGGAAACGAUGUGGGAAGGCCCAGACAGCCAGGAUGUUGGCUUAGAAGCAGCCAUCA
UUUAAAGAAAGCGUAAUAGCUCACUGGUCGAGUCGGCCUGCGCGGAAGAUGUAACGGGGCUAA
ACCAUGCACCGAAGCUGCGGCAGCGACGCUUAUGCGUUGUUGGGUAGGGGAGCGUUCUGUAAG
CCUGCGAAGGUGUGCUGUGAGGCAUGCUGGAGGUAUCAGAAGUGCGAAUGCUGACAUAAGUAA
CGAUAAAGCGGGUGAAAAGCCCGCUCGCCGGAAGACCAAGGGUUCCUGUCCAACGUUAAUCGG
GGCAGGGUGAGUCGACCCCUAAGGCGAGGCCGAAAGGCGUAGUCGAUGGGAAACAGGUUAAUA
UUCCUGUACUUGGUGUUACUGCGAAGGGGGGACGGAGAAGGCUAUGUUGGCCGGGCGACGGUU
GUCCCGGUUUAAGCGUGUAGGCUGGUUUUCCAGGCAAAUCCGGAAAAUCAAGGCUGAGGCGUG
AUGACGAGGCACUACGGUGCUGAAGCAACAAAUGCCCUGCUUCCAGGAAAAGCCUCUAAGCAU
CAGGUAACAUCAAAUCGUACCCCAAACCGACACAGGUGGUCAGGUAGAGAAUACCAAGGCGCU
UGAGAGAACUCGGGUGAAGGAACUAGGCAAAAUGGUGCCGUAACUUCGGGAGAAGGCACGCUG
AUAUGUAGGUGAGGUCCCUCGCGGAUGGAGCUGAAAUCAGUCGAAGAUACCAGCUGGCUGCAA
CUGUUUAUUAAAAACACAGCACUGUGCAAACACGAAAGUGGACGUAUACGGUGUGACGCCUGC
CCGGUGCCGGAAGGUUAAUUGAUGGGGUUAGCGCAAGCGAAGCUCUUGAUCGAAGCCCCGGUA
AACGGCGGCCGUAACUAUAACGGUCCUAAGGUAGCGAAAUUCCUUGUCGGGUAAGUUCCGACC
UGCACGAAUGGCGUAAUGAUGGCCAGGCUGUCUCCACCCGAGACUCAGUGAAAUUGAACUCGC
UGUGAAGAUGCAGUGUACCCGCGGCAAGACGGAAAGACCCCGUGAACCUUUACUAUAGCUUGA
CACUGAACAUUGAGCCUUGAUGUGUAGGAUAGGUGGGAGGCUUUGAAGUGUGGACGCCAGUCU
GCAUGGAGCCGAGCUUGAAAUACCACCCUUUAAUGUUUGAUGUUCUAACGUUGACCCGUAAUC
CGGGUUGCGGACAGUGUCUGGUGGGUAGUUUGACUGGGGCGGUCUCCUCCUAAAGAGUAACGG
AGGAGCACGAAGGUUGGCUAAUCCUGGUCGGACAUCAGGAGGUUAGUGCAAUGGCAUAAGCCA
GCUUGACUGCGAGCGUGACGGCGCGAGCAGGUGCGAAAGCAGGUCAUAGUGAUCCGGUGGUUC
UGAAUGGAAGGGCCAUCGCUCAACGGAUAAAAGGUACUCCGGGGAUAACAGGCUGAUACCGCC
CAAGAGUUCAUAUCGACGGCGGUGUUUGGCACCUCGAUGUCGGCUCAUCACAUCCUGGGGCUG
AAGUAGGUCCCAAGGGUAUGGCUGUUCGCCAUUUAAAGUGGUACGCGAGCUGGGUUUAGAACG
UCGUGAGACAGUUCGGUCCCUAUCUGCCGUGGGCGCUGGAGAACUGAGGGGGGCUGCUCCUAG
UACGAGAGGACCGGAGUGGACGCAUCACUGGUGUUCGGGUUGUCAUGCCAAUGGCACUGCCCG
GUAGCUAAAUGCGGAAGAGAUAAGUGCUGAAAGCAUCUAAGCACGAAACUUGCCCCGAGAUGA
GUUCUCCCUGACCCUUUAAGGGUCCUGAAGGAACGUUGAAGACGACGACGUUGAUAGGCCGGG
UGUGUAAGGACGACCUUCGGGAGGGCGCUUACCACUUUGUGAUUCAUGACUGGGGUGAAGUCG
UAACAAGGUAACCGUAGGGGAACCUGCGGUUGGAUCACUGUGGUA
Pair 3: Full Sequence (SEQ ID NO: 3)
c
Pair 4: Full Sequence (SEQ ID NO: 4)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
AGUCAAUAAGUCUUGAGCUAACCGGUACUAAUGAACCGUGAGGCUUAACCGAGAGGUUAAGCG
ACUAAGCGUACACGGUGGAUGCCCUGGCAGUCAGAGGCGAUGAAGGACGUGCUAAUCUGCGAU
AAGCGUCGGUAAGGUGAUAUGAACCGUUAUAACCGGCGAUUUCCGAAUGGGGAAACCCAGUGU
GUUUCGACACACUAUCAUUAACUGAAUCCAUAGGUUAAUGAGGCGAACCGGGGGAACUGAAAC
AUCUAAGUACCCCGAGGAAAAGAAAUCAACCGAGAUUCCCCCAGUAGCGGCGAGCGAACGGGG
AGCAGCCCAGAGCCUGAAUCAGUGUGUGUGUUAGUGGAAGCGUCUGGAAAGGCGCGCGAUACA
GGGUGACAGCCCCGUACACAAAAAUGCACAUGCUGUGAGCUCGAUGAGUAGGGCGGGACACGU
GGUAUCCUGUCUGAAUAUGGGGGGACCAUCCUCCAAGGCUAAAUACUCCUGACUGACCGAUAG
UGAACCAGUACCGUGAGGGAAAGGCGAAAAGAACCCCGGCGAGGGGAGUGAAAAAGAACCUGA
AACCGUGUACGUACAAGCAGUGGGAGCACGCUUAGGCGUGUGACUGCGUACCUUUUGUAUAAU
GGGUCAGCGACUUAUAUUCUGUAGCAAGGUUAACCGAAUAGGGGAGCCGAAGGGAAACCGAGU
CUUAACUGGGCGUUAAGUUGCAGGGUAUAGACCCGAAACCCGGUGAUCUAGCCAUGGGCAGGU
UGAAGGUUGGGUAACACUAACUGGAGGACCGAACCGACUAAUGUUGAAAAAUUAGCGGAUGAC
UUGUGGCUGGGGGUGAAAGGCCAAUCAAACCGGGAGAUAGCUGGUUCUCCCCGAAAGCUAUUU
AGGUAGCGCCUCGUGAAUUCAUCUCCGGGGGUAGAGCACUGUUUCGGCAAGGGGGUCAUCCCG
ACUUACCAACCCGAUGCAAACUGCGAAUACCGGAGAAUGUUAUCACGGGAGACACACGGCGGG
UGCUAACGUCCGUCGUGAAGAGGGAAACAACCCAGACCGCCAGCUAAGGUCCCAAAGUCAUGG
UUAAGUGGGAAACGAUGUGGGAAGGCCCAGACAGCCAGGAUGUUGGCUUAGAAGCAGCCAUCA
UUUAAAGAAAGCGUAAUAGCUCACUGGUCGAGUCGGCCUGCGCGGAAGAUGUAACGGGGCUAA
ACCAUGCACCGAAGCUGCGGCAGCGACGCUUAUGCGUUGUUGGGUAGGGGAGCGUUCUGUAAG
CCUGCGAAGGUGUGCUGUGAGGCAUGCUGGAGGUAUCAGAAGUGCGAAUGCUGACAUAAGUAA
CGAUAAAGCGGGUGAAAAGCCCGCUCGCCGGAAGACCAAGGGUUCCUGUCCAACGUUAAUCGG
GGCAGGGUGAGUCGACCCCUAAGGCGAGGCCGAAAGGCGUAGUCGAUGGGAAACAGGUUAAUA
UUCCUGUACUUGGUGUUACUGCGAAGGGGGGACGGAGAAGGCUAUGUUGGCCGGGCGACGGUU
GUCCCGGUUUAAGCGUGUAGGCUGGUUUUCCAGGCAAAUCCGGAAAAUCAAGGCUGAGGCGUG
AUGACGAGGCACUACGGUGCUGAAGCAACAAAUGCCCUGCUUCCAGGAAAAGCCUCUAAGCAU
CAGGUAACAUCAAAUCGUACCCCAAACCGACACAGGUGGUCAGGUAGAGAAUACCAAGGCGCU
UGAGAGAACUCGGGUGAAGGAACUAGGCAAAAUGGUGCCGUAACUUCGGGAGAAGGCACGCUG
AUAUGUAGGUGAGGUCCCUCGCGGAUGGAGCUGAAAUCAGUCGAAGAUACCAGCUGGCUGCAA
CUGUUUAUUAAAAACACAGCACUGUGCAAACACGAAAGUGGACGUAUACGGUGUGACGCCUGC
CCGGUGCCGGAAGGUUAAUUGAUGGGGUUAGCGCAAGCGAAGCUCUUGAUCGAAGCCCCGGUA
AACGGCGGCCGUAACUAUAACGGUCCUAAGGUAGCGAAAUUCCUUGUCGGGUAAGUUCCGACC
UGCACGAAUGGCGUAAUGAUGGCCAGGCUGUCUCCACCCGAGACUCAGUGAAAUUGAACUCGC
UGUGAAGAUGCAGUGUACCCGCGGCAAGACGGAAAGACCCCGUGAACCUUUACUAUAGCUUGA
CACUGAACAUUGAGCCUUGAUGUGUAGGAUAGGUGGGAGGCUUUGAAGUGUGGACGCCAGUCU
GCAUGGAGCCGAGCUUGAAAUACCACCCUUUAAUGUUUGAUGUUCUAACGUUGACCCGUAAUC
CGGGUUGCGGACAGUGUCUGGUGGGUAGUUUGACUGGGGCGGUCUCCUCCUAAAGAGUAACGG
AGGAGCACGAAGGUUGGCUAAUCCUGGUCGGACAUCAGGAGGUUAGUGCAAUGGCAUAAGCCA
GCUUGACUGCGAGCGUGACGGCGCGAGCAGGUGCGAAAGCAGGUCAUAGUGAUCCGGUGGUUC
UGAAUGGAAGGGCCAUCGCUCAACGGAUAAAAGGUACUCCGGGGAUAACAGGCUGAUACCGCC
CAAGAGUUCAUAUCGACGGCGGUGUUUGGCACCUCGAUGUCGGCUCAUCACAUCCUGGGGCUG
AAGUAGGUCCCAAGGGUAUGGCUGUUCGCCAUUUAAAGUGGUACGCGAGCUGGGUUUAGAACG
UCGUGAGACAGUUCGGUCCCUAUCUGCCGUGGGCGCUGGAGAACUGAGGGGGGCUGCUCCUAG
UACGAGAGGACCGGAGUGGACGCAUCACUGGUGUUCGGGUUGUCAUGCCAAUGGCACUGCCCG
GUAGCUAAAUGCGGAAGAGAUAAGUGCUGAAAGCAUCUAAGCACGAAACUUGCCCCGAGAUGA
GUUCUCCCUGACCCUUUAAGGGUCCUGAAGGAACGUUGAAGACGACGACGUUGAUAGGCCGGG
UGUGUAAGGACUCACAACGGAGGGCGCUUACCACUUUGUGAUUCAUGACUGGGGUGAAGUCGU
AACAAGGUAACCGUAGGGGAACCUGCGGUUGGAUCACUGUGGUA
Pair 5: Full Sequence (SEQ ID NO: 5)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
AUAUAAUGUCUUGAGCUAACCGGUACUAAUGAACCGUGAGGCUUAACCGAGAGGUUAAGCGAC
UAAGCGUACACGGUGGAUGCCCUGGCAGUCAGAGGCGAUGAAGGACGUGCUAAUCUGCGAUAA
GCGUCGGUAAGGUGAUAUGAACCGUUAUAACCGGCGAUUUCCGAAUGGGGAAACCCAGUGUGU
UUCGACACACUAUCAUUAACUGAAUCCAUAGGUUAAUGAGGCGAACCGGGGGAACUGAAACAU
CUAAGUACCCCGAGGAAAAGAAAUCAACCGAGAUUCCCCCAGUAGCGGCGAGCGAACGGGGAG
CAGCCCAGAGCCUGAAUCAGUGUGUGUGUUAGUGGAAGCGUCUGGAAAGGCGCGCGAUACAGG
GUGACAGCCCCGUACACAAAAAUGCACAUGCUGUGAGCUCGAUGAGUAGGGCGGGACACGUGG
UAUCCUGUCUGAAUAUGGGGGGACCAUCCUCCAAGGCUAAAUACUCCUGACUGACCGAUAGUG
AACCAGUACCGUGAGGGAAAGGCGAAAAGAACCCCGGCGAGGGGAGUGAAAAAGAACCUGAAA
CCGUGUACGUACAAGCAGUGGGAGCACGCUUAGGCGUGUGACUGCGUACCUUUUGUAUAAUGG
GUCAGCGACUUAUAUUCUGUAGCAAGGUUAACCGAAUAGGGGAGCCGAAGGGAAACCGAGUCU
UAACUGGGCGUUAAGUUGCAGGGUAUAGACCCGAAACCCGGUGAUCUAGCCAUGGGCAGGUUG
AAGGUUGGGUAACACUAACUGGAGGACCGAACCGACUAAUGUUGAAAAAUUAGCGGAUGACUU
GUGGCUGGGGGUGAAAGGCCAAUCAAACCGGGAGAUAGCUGGUUCUCCCCGAAAGCUAUUUAG
GUAGCGCCUCGUGAAUUCAUCUCCGGGGGUAGAGCACUGUUUCGGCAAGGGGGUCAUCCCGAC
UUACCAACCCGAUGCAAACUGCGAAUACCGGAGAAUGUUAUCACGGGAGACACACGGCGGGUG
CUAACGUCCGUCGUGAAGAGGGAAACAACCCAGACCGCCAGCUAAGGUCCCAAAGUCAUGGUU
AAGUGGGAAACGAUGUGGGAAGGCCCAGACAGCCAGGAUGUUGGCUUAGAAGCAGCCAUCAUU
UAAAGAAAGCGUAAUAGCUCACUGGUCGAGUCGGCCUGCGCGGAAGAUGUAACGGGGCUAAAC
CAUGCACCGAAGCUGCGGCAGCGACGCUUAUGCGUUGUUGGGUAGGGGAGCGUUCUGUAAGCC
UGCGAAGGUGUGCUGUGAGGCAUGCUGGAGGUAUCAGAAGUGCGAAUGCUGACAUAAGUAACG
AUAAAGCGGGUGAAAAGCCCGCUCGCCGGAAGACCAAGGGUUCCUGUCCAACGUUAAUCGGGG
CAGGGUGAGUCGACCCCUAAGGCGAGGCCGAAAGGCGUAGUCGAUGGGAAACAGGUUAAUAUU
CCUGUACUUGGUGUUACUGCGAAGGGGGGACGGAGAAGGCUAUGUUGGCCGGGCGACGGUUGU
CCCGGUUUAAGCGUGUAGGCUGGUUUUCCAGGCAAAUCCGGAAAAUCAAGGCUGAGGCGUGAU
GACGAGGCACUACGGUGCUGAAGCAACAAAUGCCCUGCUUCCAGGAAAAGCCUCUAAGCAUCA
GGUAACAUCAAAUCGUACCCCAAACCGACACAGGUGGUCAGGUAGAGAAUACCAAGGCGCUUG
AGAGAACUCGGGUGAAGGAACUAGGCAAAAUGGUGCCGUAACUUCGGGAGAAGGCACGCUGAU
AUGUAGGUGAGGUCCCUCGCGGAUGGAGCUGAAAUCAGUCGAAGAUACCAGCUGGCUGCAACU
GUUUAUUAAAAACACAGCACUGUGCAAACACGAAAGUGGACGUAUACGGUGUGACGCCUGCCC
GGUGCCGGAAGGUUAAUUGAUGGGGUUAGCGCAAGCGAAGCUCUUGAUCGAAGCCCCGGUAAA
CGGCGGCCGUAACUAUAACGGUCCUAAGGUAGCGAAAUUCCUUGUCGGGUAAGUUCCGACCUG
CACGAAUGGCGUAAUGAUGGCCAGGCUGUCUCCACCCGAGACUCAGUGAAAUUGAACUCGCUG
UGAAGAUGCAGUGUACCCGCGGCAAGACGGAAAGACCCCGUGAACCUUUACUAUAGCUUGACA
CUGAACAUUGAGCCUUGAUGUGUAGGAUAGGUGGGAGGCUUUGAAGUGUGGACGCCAGUCUGC
AUGGAGCCGAGCUUGAAAUACCACCCUUUAAUGUUUGAUGUUCUAACGUUGACCCGUAAUCCG
GGUUGCGGACAGUGUCUGGUGGGUAGUUUGACUGGGGCGGUCUCCUCCUAAAGAGUAACGGAG
GAGCACGAAGGUUGGCUAAUCCUGGUCGGACAUCAGGAGGUUAGUGCAAUGGCAUAAGCCAGC
UUGACUGCGAGCGUGACGGCGCGAGCAGGUGCGAAAGCAGGUCAUAGUGAUCCGGUGGUUCUG
AAUGGAAGGGCCAUCGCUCAACGGAUAAAAGGUACUCCGGGGAUAACAGGCUGAUACCGCCCA
AGAGUUCAUAUCGACGGCGGUGUUUGGCACCUCGAUGUCGGCUCAUCACAUCCUGGGGCUGAA
GUAGGUCCCAAGGGUAUGGCUGUUCGCCAUUUAAAGUGGUACGCGAGCUGGGUUUAGAACGUC
GUGAGACAGUUCGGUCCCUAUCUGCCGUGGGCGCUGGAGAACUGAGGGGGGCUGCUCCUAGUA
CGAGAGGACCGGAGUGGACGCAUCACUGGUGUUCGGGUUGUCAUGCCAAUGGCACUGCCCGGU
AGCUAAAUGCGGAAGAGAUAAGUGCUGAAAGCAUCUAAGCACGAAACUUGCCCCGAGAUGAGU
UCUCCCUGACCCUUUAAGGGUCCUGAAGGAACGUUGAAGACGACGACGUUGAUAGGCCGGGUG
UGUAAGGACGACCUUCGGGAGGGCGCUUACCACUUUGUGAUUCAUGACUGGGGUGAAGUCGUA
ACAAGGUAACCGUAGGGGAACCUGCGGUUGGAUCACUGUGGUA
Pair 6: Full Sequence (SEQ ID NO: 6)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
AUAUAAUGUCUUGAGCUAACCGGUACUAAUGAACCGUGAGGCUUAACCGAGAGGUUAAGCGAC
UAAGCGUACACGGUGGAUGCCCUGGCAGUCAGAGGCGAUGAAGGACGUGCUAAUCUGCGAUAA
GCGUCGGUAAGGUGAUAUGAACCGUUAUAACCGGCGAUUUCCGAAUGGGGAAACCCAGUGUGU
UUCGACACACUAUCAUUAACUGAAUCCAUAGGUUAAUGAGGCGAACCGGGGGAACUGAAACAU
CUAAGUACCCCGAGGAAAAGAAAUCAACCGAGAUUCCCCCAGUAGCGGCGAGCGAACGGGGAG
CAGCCCAGAGCCUGAAUCAGUGUGUGUGUUAGUGGAAGCGUCUGGAAAGGCGCGCGAUACAGG
GUGACAGCCCCGUACACAAAAAUGCACAUGCUGUGAGCUCGAUGAGUAGGGCGGGACACGUGG
UAUCCUGUCUGAAUAUGGGGGGACCAUCCUCCAAGGCUAAAUACUCCUGACUGACCGAUAGUG
AACCAGUACCGUGAGGGAAAGGCGAAAAGAACCCCGGCGAGGGGAGUGAAAAAGAACCUGAAA
CCGUGUACGUACAAGCAGUGGGAGCACGCUUAGGCGUGUGACUGCGUACCUUUUGUAUAAUGG
GUCAGCGACUUAUAUUCUGUAGCAAGGUUAACCGAAUAGGGGAGCCGAAGGGAAACCGAGUCU
UAACUGGGCGUUAAGUUGCAGGGUAUAGACCCGAAACCCGGUGAUCUAGCCAUGGGCAGGUUG
AAGGUUGGGUAACACUAACUGGAGGACCGAACCGACUAAUGUUGAAAAAUUAGCGGAUGACUU
GUGGCUGGGGGUGAAAGGCCAAUCAAACCGGGAGAUAGCUGGUUCUCCCCGAAAGCUAUUUAG
GUAGCGCCUCGUGAAUUCAUCUCCGGGGGUAGAGCACUGUUUCGGCAAGGGGGUCAUCCCGAC
UUACCAACCCGAUGCAAACUGCGAAUACCGGAGAAUGUUAUCACGGGAGACACACGGCGGGUG
CUAACGUCCGUCGUGAAGAGGGAAACAACCCAGACCGCCAGCUAAGGUCCCAAAGUCAUGGUU
AAGUGGGAAACGAUGUGGGAAGGCCCAGACAGCCAGGAUGUUGGCUUAGAAGCAGCCAUCAUU
UAAAGAAAGCGUAAUAGCUCACUGGUCGAGUCGGCCUGCGCGGAAGAUGUAACGGGGCUAAAC
CAUGCACCGAAGCUGCGGCAGCGACGCUUAUGCGUUGUUGGGUAGGGGAGCGUUCUGUAAGCC
UGCGAAGGUGUGCUGUGAGGCAUGCUGGAGGUAUCAGAAGUGCGAAUGCUGACAUAAGUAACG
AUAAAGCGGGUGAAAAGCCCGCUCGCCGGAAGACCAAGGGUUCCUGUCCAACGUUAAUCGGGG
CAGGGUGAGUCGACCCCUAAGGCGAGGCCGAAAGGCGUAGUCGAUGGGAAACAGGUUAAUAUU
CCUGUACUUGGUGUUACUGCGAAGGGGGGACGGAGAAGGCUAUGUUGGCCGGGCGACGGUUGU
CCCGGUUUAAGCGUGUAGGCUGGUUUUCCAGGCAAAUCCGGAAAAUCAAGGCUGAGGCGUGAU
GACGAGGCACUACGGUGCUGAAGCAACAAAUGCCCUGCUUCCAGGAAAAGCCUCUAAGCAUCA
GGUAACAUCAAAUCGUACCCCAAACCGACACAGGUGGUCAGGUAGAGAAUACCAAGGCGCUUG
AGAGAACUCGGGUGAAGGAACUAGGCAAAAUGGUGCCGUAACUUCGGGAGAAGGCACGCUGAU
AUGUAGGUGAGGUCCCUCGCGGAUGGAGCUGAAAUCAGUCGAAGAUACCAGCUGGCUGCAACU
GUUUAUUAAAAACACAGCACUGUGCAAACACGAAAGUGGACGUAUACGGUGUGACGCCUGCCC
GGUGCCGGAAGGUUAAUUGAUGGGGUUAGCGCAAGCGAAGCUCUUGAUCGAAGCCCCGGUAAA
CGGCGGCCGUAACUAUAACGGUCCUAAGGUAGCGAAAUUCCUUGUCGGGUAAGUUCCGACCUG
CACGAAUGGCGUAAUGAUGGCCAGGCUGUCUCCACCCGAGACUCAGUGAAAUUGAACUCGCUG
UGAAGAUGCAGUGUACCCGCGGCAAGACGGAAAGACCCCGUGAACCUUUACUAUAGCUUGACA
CUGAACAUUGAGCCUUGAUGUGUAGGAUAGGUGGGAGGCUUUGAAGUGUGGACGCCAGUCUGC
AUGGAGCCGAGCUUGAAAUACCACCCUUUAAUGUUUGAUGUUCUAACGUUGACCCGUAAUCCG
GGUUGCGGACAGUGUCUGGUGGGUAGUUUGACUGGGGCGGUCUCCUCCUAAAGAGUAACGGAG
GAGCACGAAGGUUGGCUAAUCCUGGUCGGACAUCAGGAGGUUAGUGCAAUGGCAUAAGCCAGC
UUGACUGCGAGCGUGACGGCGCGAGCAGGUGCGAAAGCAGGUCAUAGUGAUCCGGUGGUUCUG
AAUGGAAGGGCCAUCGCUCAACGGAUAAAAGGUACUCCGGGGAUAACAGGCUGAUACCGCCCA
AGAGUUCAUAUCGACGGCGGUGUUUGGCACCUCGAUGUCGGCUCAUCACAUCCUGGGGCUGAA
GUAGGUCCCAAGGGUAUGGCUGUUCGCCAUUUAAAGUGGUACGCGAGCUGGGUUUAGAACGUC
GUGAGACAGUUCGGUCCCUAUCUGCCGUGGGCGCUGGAGAACUGAGGGGGGCUGCUCCUAGUA
CGAGAGGACCGGAGUGGACGCAUCACUGGUGUUCGGGUUGUCAUGCCAAUGGCACUGCCCGGU
AGCUAAAUGCGGAAGAGAUAAGUGCUGAAAGCAUCUAAGCACGAAACUUGCCCCGAGAUGAGU
UCUCCCUGACCCUUUAAGGGUCCUGAAGGAACGUUGAAGACGACGACGUUGAUAGGCCGGGUG
UGUAAGGACUCACAACGGAGGGCGCUUACCACUUUGUGAUUCAUGACUGGGGUGAAGUCGUAA
CAAGGUAACCGUAGGGGAACCUGCGGUUGGAUCACUGUGGUA
Pair 7: Full Sequence (SEQ ID NO: 7)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
GUUAUAGUCUUGAGCUAACCGGUACUAAUGAACCGUGAGGCUUAACCGAGAGGUUAAGCGACU
AAGCGUACACGGUGGAUGCCCUGGCAGUCAGAGGCGAUGAAGGACGUGCUAAUCUGCGAUAAG
CGUCGGUAAGGUGAUAUGAACCGUUAUAACCGGCGAUUUCCGAAUGGGGAAACCCAGUGUGUU
UCGACACACUAUCAUUAACUGAAUCCAUAGGUUAAUGAGGCGAACCGGGGGAACUGAAACAUC
UAAGUACCCCGAGGAAAAGAAAUCAACCGAGAUUCCCCCAGUAGCGGCGAGCGAACGGGGAGC
AGCCCAGAGCCUGAAUCAGUGUGUGUGUUAGUGGAAGCGUCUGGAAAGGCGCGCGAUACAGGG
UGACAGCCCCGUACACAAAAAUGCACAUGCUGUGAGCUCGAUGAGUAGGGCGGGACACGUGGU
AUCCUGUCUGAAUAUGGGGGGACCAUCCUCCAAGGCUAAAUACUCCUGACUGACCGAUAGUGA
ACCAGUACCGUGAGGGAAAGGCGAAAAGAACCCCGGCGAGGGGAGUGAAAAAGAACCUGAAAC
CGUGUACGUACAAGCAGUGGGAGCACGCUUAGGCGUGUGACUGCGUACCUUUUGUAUAAUGGG
UCAGCGACUUAUAUUCUGUAGCAAGGUUAACCGAAUAGGGGAGCCGAAGGGAAACCGAGUCUU
AACUGGGCGUUAAGUUGCAGGGUAUAGACCCGAAACCCGGUGAUCUAGCCAUGGGCAGGUUGA
AGGUUGGGUAACACUAACUGGAGGACCGAACCGACUAAUGUUGAAAAAUUAGCGGAUGACUUG
UGGCUGGGGGUGAAAGGCCAAUCAAACCGGGAGAUAGCUGGUUCUCCCCGAAAGCUAUUUAGG
UAGCGCCUCGUGAAUUCAUCUCCGGGGGUAGAGCACUGUUUCGGCAAGGGGGUCAUCCCGACU
UACCAACCCGAUGCAAACUGCGAAUACCGGAGAAUGUUAUCACGGGAGACACACGGCGGGUGC
UAACGUCCGUCGUGAAGAGGGAAACAACCCAGACCGCCAGCUAAGGUCCCAAAGUCAUGGUUA
AGUGGGAAACGAUGUGGGAAGGCCCAGACAGCCAGGAUGUUGGCUUAGAAGCAGCCAUCAUUU
AAAGAAAGCGUAAUAGCUCACUGGUCGAGUCGGCCUGCGCGGAAGAUGUAACGGGGCUAAACC
AUGCACCGAAGCUGCGGCAGCGACGCUUAUGCGUUGUUGGGUAGGGGAGCGUUCUGUAAGCCU
GCGAAGGUGUGCUGUGAGGCAUGCUGGAGGUAUCAGAAGUGCGAAUGCUGACAUAAGUAACGA
UAAAGCGGGUGAAAAGCCCGCUCGCCGGAAGACCAAGGGUUCCUGUCCAACGUUAAUCGGGGC
AGGGUGAGUCGACCCCUAAGGCGAGGCCGAAAGGCGUAGUCGAUGGGAAACAGGUUAAUAUUC
CUGUACUUGGUGUUACUGCGAAGGGGGGACGGAGAAGGCUAUGUUGGCCGGGCGACGGUUGUC
CCGGUUUAAGCGUGUAGGCUGGUUUUCCAGGCAAAUCCGGAAAAUCAAGGCUGAGGCGUGAUG
ACGAGGCACUACGGUGCUGAAGCAACAAAUGCCCUGCUUCCAGGAAAAGCCUCUAAGCAUCAG
GUAACAUCAAAUCGUACCCCAAACCGACACAGGUGGUCAGGUAGAGAAUACCAAGGCGCUUGA
GAGAACUCGGGUGAAGGAACUAGGCAAAAUGGUGCCGUAACUUCGGGAGAAGGCACGCUGAUA
UGUAGGUGAGGUCCCUCGCGGAUGGAGCUGAAAUCAGUCGAAGAUACCAGCUGGCUGCAACUG
UUUAUUAAAAACACAGCACUGUGCAAACACGAAAGUGGACGUAUACGGUGUGACGCCUGCCCG
GUGCCGGAAGGUUAAUUGAUGGGGUUAGCGCAAGCGAAGCUCUUGAUCGAAGCCCCGGUAAAC
GGCGGCCGUAACUAUAACGGUCCUAAGGUAGCGAAAUUCCUUGUCGGGUAAGUUCCGACCUGC
ACGAAUGGCGUAAUGAUGGCCAGGCUGUCUCCACCCGAGACUCAGUGAAAUUGAACUCGCUGU
GAAGAUGCAGUGUACCCGCGGCAAGACGGAAAGACCCCGUGAACCUUUACUAUAGCUUGACAC
UGAACAUUGAGCCUUGAUGUGUAGGAUAGGUGGGAGGCUUUGAAGUGUGGACGCCAGUCUGCA
UGGAGCCGAGCUUGAAAUACCACCCUUUAAUGUUUGAUGUUCUAACGUUGACCGGUAAUCCGG
GUUGCGGACAGUGUCUGGUGGGUAGUUUGACUGGGGCGGUCUCCUCCUAAAGAGUAACGGAGG
AGCACGAAGGUUGGCUAAUCCUGGUCGGACAUCAGGAGGUUAGUGCAAUGGCAUAAGCCAGCU
UGACUGCGAGCGUGACGGCGCGAGCAGGUGCGAAAGCAGGUCAUAGUGAUCCGGUGGUUCUGA
AUGGAAGGGCCAUCGCUCAACGGAUAAAAGGUACUCCGGGGAUAACAGGCUGAUACCGCCCAA
GAGUUCAUAUCGACGGCGGUGUUUGGCACCUCGAUGUCGGCUCAUCACAUCCUGGGGCUGAAG
UAGGUCCCAAGGGUAUGGCUGUUCGCCAUUUAAAGUGGUACGCGAGCUGGGUUUAGAACGUCG
UGAGACAGUUCGGUCCCUAUCUGCCGUGGGCGCUGGAGAACUGAGGGGGGCUGCUCCUAGUAC
GAGAGGACCGGAGUGGACGCAUCACUGGUGUUCGGGUUGUCAUGCCAAUGGCACUGCCCGGUA
GCUAAAUGCGGAAGAGAUAAGUGCUGAAAGCAUCUAAGCACGAAACUUGCCCCGAGAUGAGUU
CUCCCUGACCCUUUAAGGGUCCUGAAGGAACGUUGAAGACGACGACGUUGAUAGGCCGGGUGU
GUAAGGACAUCCCAGGGGAGGGCGCUUACCACUUUGUGAUUCAUGACUGGGGUGAAGUCGUAA
CAAGGUAACCGUAGGGGAACCUGCGGUUGGAUCACUGUGGUA
Pair 8: Full Sequence (SEQ ID NO: 8)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
CAUCAUGGGUCUUGAGCUAACCGGUACUAAUGAACCGUGAGGCUUAACCGAGAGGUUAAGCGA
CUAAGCGUACACGGUGGAUGCCCUGGCAGUCAGAGGCGAUGAAGGACGUGCUAAUCUGCGAUA
AGCGUCGGUAAGGUGAUAUGAACCGUUAUAACCGGCGAUUUCCGAAUGGGGAAACCCAGUGUG
UUUCGACACACUAUCAUUAACUGAAUCCAUAGGUUAAUGAGGCGAACCGGGGGAACUGAAACA
UCUAAGUACCCCGAGGAAAAGAAAUCAACCGAGAUUCCCCCAGUAGCGGCGAGCGAACGGGGA
GCAGCCCAGAGCCUGAAUCAGUGUGUGUGUUAGUGGAAGCGUCUGGAAAGGCGCGCGAUACAG
GGUGACAGCCCCGUACACAAAAAUGCACAUGCUGUGAGCUCGAUGAGUAGGGCGGGACACGUG
GUAUCCUGUCUGAAUAUGGGGGGACCAUCCUCCAAGGCUAAAUACUCCUGACUGACCGAUAGU
GAACCAGUACCGUGAGGGAAAGGCGAAAAGAACCCCGGCGAGGGGAGUGAAAAAGAACCUGAA
ACCGUGUACGUACAAGCAGUGGGAGCACGCUUAGGCGUGUGACUGCGUACCUUUUGUAUAAUG
GGUCAGCGACUUAUAUUCUGUAGCAAGGUUAACCGAAUAGGGGAGCCGAAGGGAAACCGAGUC
UUAACUGGGCGUUAAGUUGCAGGGUAUAGACCCGAAACCCGGUGAUCUAGCCAUGGGCAGGUU
GAAGGUUGGGUAACACUAACUGGAGGACCGAACCGACUAAUGUUGAAAAAUUAGCGGAUGACU
UGUGGCUGGGGGUGAAAGGCCAAUCAAACCGGGAGAUAGCUGGUUCUCCCCGAAAGCUAUUUA
GGUAGCGCCUCGUGAAUUCAUCUCCGGGGGUAGAGCACUGUUUCGGCAAGGGGGUCAUCCCGA
CUUACCAACCCGAUGCAAACUGCGAAUACCGGAGAAUGUUAUCACGGGAGACACACGGCGGGU
GCUAACGUCCGUCGUGAAGAGGGAAACAACCCAGACCGCCAGCUAAGGUCCCAAAGUCAUGGU
UAAGUGGGAAACGAUGUGGGAAGGCCCAGACAGCCAGGAUGUUGGCUUAGAAGCAGCCAUCAU
UUAAAGAAAGCGUAAUAGCUCACUGGUCGAGUCGGCCUGCGCGGAAGAUGUAACGGGGCUAAA
CCAUGCACCGAAGCUGCGGCAGCGACGCUUAUGCGUUGUUGGGUAGGGGAGCGUUCUGUAAGC
CUGCGAAGGUGUGCUGUGAGGCAUGCUGGAGGUAUCAGAAGUGCGAAUGCUGACAUAAGUAAC
GAUAAAGCGGGUGAAAAGCCCGCUCGCCGGAAGACCAAGGGUUCCUGUCCAACGUUAAUCGGG
GCAGGGUGAGUCGACCCCUAAGGCGAGGCCGAAAGGCGUAGUCGAUGGGAAACAGGUUAAUAU
UCCUGUACUUGGUGUUACUGCGAAGGGGGGACGGAGAAGGCUAUGUUGGCCGGGCGACGGUUG
UCCCGGUUUAAGCGUGUAGGCUGGUUUUCCAGGCAAAUCCGGAAAAUCAAGGCUGAGGCGUGA
UGACGAGGCACUACGGUGCUGAAGCAACAAAUGCCCUGCUUCCAGGAAAAGCCUCUAAGCAUC
AGGUAACAUCAAAUCGUACCCCAAACCGACACAGGUGGUCAGGUAGAGAAUACCAAGGCGCUU
GAGAGAACUCGGGUGAAGGAACUAGGCAAAAUGGUGCCGUAACUUCGGGAGAAGGCACGCUGA
UAUGUAGGUGAGGUCCCUCGCGGAUGGAGCUGAAAUCAGUCGAAGAUACCAGCUGGCUGCAAC
UGUUUAUUAAAAACACAGCACUGUGCAAACACGAAAGUGGACGUAUACGGUGUGACGCCUGCC
CGGUGCCGGAAGGUUAAUUGAUGGGGUUAGCGCAAGCGAAGCUCUUGAUCGAAGCCCCGGUAA
ACGGCGGCCGUAACUAUAACGGUCCUAAGGUAGCGAAAUUCCUUGUCGGGUAAGUUCCGACCU
GCACGAAUGGCGUAAUGAUGGCCAGGCUGUCUCCACCCGAGACUCAGUGAAAUUGAACUCGCU
GUGAAGAUGCAGUGUACCCGCGGCAAGACGGAAAGACCCCGUGAACCUUUACUAUAGCUUGAC
ACUGAACAUUGAGCCUUGAUGUGUAGGAUAGGUGGGAGGCUUUGAAGUGUGGACGCCAGUCUG
CAUGGAGCCGAGCUUGAAAUACCACCCUUUAAUGUUUGAUGUUCUAACGUUGACCGGUAAUCC
GGGUUGCGGACAGUGUCUGGUGGGUAGUUUGACUGGGGCGGUCUCCUCCUAAAGAGUAACGGA
GGAGCACGAAGGUUGGCUAAUCCUGGUCGGACAUCAGGAGGUUAGUGCAAUGGCAUAAGCCAG
CUUGACUGCGAGCGUGACGGCGCGAGCAGGUGCGAAAGCAGGUCAUAGUGAUCCGGUGGUUCU
GAAUGGAAGGGCCAUCGCUCAACGGAUAAAAGGUACUCCGGGGAUAACAGGCUGAUACCGCCC
AAGAGUUCAUAUCGACGGCGGUGUUUGGCACCUCGAUGUCGGCUCAUCACAUCCUGGGGCUGA
AGUAGGUCCCAAGGGUAUGGCUGUUCGCCAUUUAAAGUGGUACGCGAGCUGGGUUUAGAACGU
CGUGAGACAGUUCGGUCCCUAUCUGCCGUGGGCGCUGGAGAACUGAGGGGGGCUGCUCCUAGU
ACGAGAGGACCGGAGUGGACGCAUCACUGGUGUUCGGGUUGUCAUGCCAAUGGCACUGCCCGG
UAGCUAAAUGCGGAAGAGAUAAGUGCUGAAAGCAUCUAAGCACGAAACUUGCCCCGAGAUGAG
UUCUCCCUGACCCUUUAAGGGUCCUGAAGGAACGUUGAAGACGACGACGUUGAUAGGCCGGGU
GUGUAAGGACACAUAAUGGGAGGGCGCUUACCACUUUGUGAUUCAUGACUGGGGUGAAGUCGU
AACAAGGUAACCGUAGGGGAACCUGCGGUUGGAUCACUGUGGUA
Pair 9: Full Sequence (SEQ ID NO: 9)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
AGUCAAUAAGUCUUGAGCUAACCGGUACUAAUGAACCGUGAGGCUUAACCGAGAGGUUAAGCG
ACUAAGCGUACACGGUGGAUGCCCUGGCAGUCAGAGGCGAUGAAGGACGUGCUAAUCUGCGAU
AAGCGUCGGUAAGGUGAUAUGAACCGUUAUAACCGGCGAUUUCCGAAUGGGGAAACCCAGUGU
GUUUCGACACACUAUCAUUAACUGAAUCCAUAGGUUAAUGAGGCGAACCGGGGGAACUGAAAC
AUCUAAGUACCCCGAGGAAAAGAAAUCAACCGAGAUUCCCCCAGUAGCGGCGAGCGAACGGGG
AGCAGCCCAGAGCCUGAAUCAGUGUGUGUGUUAGUGGAAGCGUCUGGAAAGGCGCGCGAUACA
GGGUGACAGCCCCGUACACAAAAAUGCACAUGCUGUGAGCUCGAUGAGUAGGGCGGGACACGU
GGUAUCCUGUCUGAAUAUGGGGGGACCAUCCUCCAAGGCUAAAUACUCCUGACUGACCGAUAG
UGAACCAGUACCGUGAGGGAAAGGCGAAAAGAACCCCGGCGAGGGGAGUGAAAAAGAACCUGA
AACCGUGUACGUACAAGCAGUGGGAGCACGCUUAGGCGUGUGACUGCGUACCUUUUGUAUAAU
GGGUCAGCGACUUAUAUUCUGUAGCAAGGUUAACCGAAUAGGGGAGCCGAAGGGAAACCGAGU
CUUAACUGGGCGUUAAGUUGCAGGGUAUAGACCCGAAACCCGGUGAUCUAGCCAUGGGCAGGU
UGAAGGUUGGGUAACACUAACUGGAGGACCGAACCGACUAAUGUUGAAAAAUUAGCGGAUGAC
UUGUGGCUGGGGGUGAAAGGCCAAUCAAACCGGGAGAUAGCUGGUUCUCCCCGAAAGCUAUUU
AGGUAGCGCCUCGUGAAUUCAUCUCCGGGGGUAGAGCACUGUUUCGGCAAGGGGGUCAUCCCG
ACUUACCAACCCGAUGCAAACUGCGAAUACCGGAGAAUGUUAUCACGGGAGACACACGGCGGG
UGCUAACGUCCGUCGUGAAGAGGGAAACAACCCAGACCGCCAGCUAAGGUCCCAAAGUCAUGG
UUAAGUGGGAAACGAUGUGGGAAGGCCCAGACAGCCAGGAUGUUGGCUUAGAAGCAGCCAUCA
UUUAAAGAAAGCGUAAUAGCUCACUGGUCGAGUCGGCCUGCGCGGAAGAUGUAACGGGGCUAA
ACCAUGCACCGAAGCUGCGGCAGCGACGCUUAUGCGUUGUUGGGUAGGGGAGCGUUCUGUAAG
CCUGCGAAGGUGUGCUGUGAGGCAUGCUGGAGGUAUCAGAAGUGCGAAUGCUGACAUAAGUAA
CGAUAAAGCGGGUGAAAAGCCCGCUCGCCGGAAGACCAAGGGUUCCUGUCCAACGUUAAUCGG
GGCAGGGUGAGUCGACCCCUAAGGCGAGGCCGAAAGGCGUAGUCGAUGGGAAACAGGUUAAUA
UUCCUGUACUUGGUGUUACUGCGAAGGGGGGACGGAGAAGGCUAUGUUGGCCGGGCGACGGUU
GUCCCGGUUUAAGCGUGUAGGCUGGUUUUCCAGGCAAAUCCGGAAAAUCAAGGCUGAGGCGUG
AUGACGAGGCACUACGGUGCUGAAGCAACAAAUGCCCUGCUUCCAGGAAAAGCCUCUAAGCAU
CAGGUAACAUCAAAUCGUACCCCAAACCGACACAGGUGGUCAGGUAGAGAAUACCAAGGCGCU
UGAGAGAACUCGGGUGAAGGAACUAGGCAAAAUGGUGCCGUAACUUCGGGAGAAGGCACGCUG
AUAUGUAGGUGAGGUCCCUCGCGGAUGGAGCUGAAAUCAGUCGAAGAUACCAGCUGGCUGCAA
CUGUUUAUUAAAAACACAGCACUGUGCAAACACGAAAGUGGACGUAUACGGUGUGACGCCUGC
CCGGUGCCGGAAGGUUAAUUGAUGGGGUUAGCGCAAGCGAAGCUCUUGAUCGAAGCCCCGGUA
AACGGCGGCCGUAACUAUAACGGUCCUAAGGUAGCGAAAUUCCUUGUCGGGUAAGUUCCGACC
UGCACGAAUGGCGUAAUGAUGGCCAGGCUGUCUCCACCCGAGACUCAGUGAAAUUGAACUCGC
UGUGAAGAUGCAGUGUACCCGCGGCAAGACGGAAAGACCCCGUGAACCUUUACUAUAGCUUGA
CACUGAACAUUGAGCCUUGAUGUGUAGGAUAGGUGGGAGGCUUUGAAGUGUGGACGCCAGUCU
GCAUGGAGCCGAGCUUGAAAUACCACCCUUUAAUGUUUGAUGUUCUAACGUUGACCCGUAAUC
CGGGUUGCGGACAGUGUCUGGUGGGUAGUUUGACUGGGGCGGUCUCCUCCUAAAGAGUAACGG
AGGAGCACGAAGGUUGGCUAAUCCUGGUCGGACAUCAGGAGGUUAGUGCAAUGGCAUAAGCCA
GCUUGACUGCGAGCGUGACGGCGCGAGCAGGUGCGAAAGCAGGUCAUAGUGAUCCGGUGGUUC
UGAAUGGAAGGGCCAUCGCUCAACGGAUAAAAGGUACUCCGGGGAUAACAGGCUGAUACCGCC
CAAGAGUUCAUAUCGACGGCGGUGUUUGGCACCUCGAUGUCGGCUCAUCACAUCCUGGGGCUG
AAGUAGGUCCCAAGGGUAUGGCUGUUCGCCAUUUAAAGUGGUACGCGAGCUGGGUUUAGAACG
UCGUGAGACAGUUCGGUCCCUAUCUGCCGUGGGCGCUGGAGAACUGAGGGGGGCUGCUCCUAG
UACGAGAGGACCGGAGUGGACGCAUCACUGGUGUUCGGGUUGUCAUGCCAAUGGCACUGCCCG
GUAGCUAAAUGCGGAAGAGAUAAGUGCUGAAAGCAUCUAAGCACGAAACUUGCCCCGAGAUGA
GUUCUCCCUGACCCUUUAAGGGUCCUGAAGGAACGUUGAAGACGACGACGUUGAUAGGCCGGG
UGUGUAAGGACAUCCCAGGGGAGGGCGCUUACCACUUUGUGAUUCAUGACUGGGGUGAAGUCG
UAACAAGGUAACCGUAGGGGAACCUGCGGUUGGAUCACUGUGGUA
Pair 10: Full Sequence (SEQ ID NO: 10)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
CAUCAUGGGUCUUGAGCUAACCGGUACUAAUGAACCGUGAGGCUUAACCGAGAGGUUAAGCGA
CUAAGCGUACACGGUGGAUGCCCUGGCAGUCAGAGGCGAUGAAGGACGUGCUAAUCUGCGAUA
AGCGUCGGUAAGGUGAUAUGAACCGUUAUAACCGGCGAUUUCCGAAUGGGGAAACCCAGUGUG
UUUCGACACACUAUCAUUAACUGAAUCCAUAGGUUAAUGAGGCGAACCGGGGGAACUGAAACA
UCUAAGUACCCCGAGGAAAAGAAAUCAACCGAGAUUCCCCCAGUAGCGGCGAGCGAACGGGGA
GCAGCCCAGAGCCUGAAUCAGUGUGUGUGUUAGUGGAAGCGUCUGGAAAGGCGCGCGAUACAG
GGUGACAGCCCCGUACACAAAAAUGCACAUGCUGUGAGCUCGAUGAGUAGGGCGGGACACGUG
GUAUCCUGUCUGAAUAUGGGGGGACCAUCCUCCAAGGCUAAAUACUCCUGACUGACCGAUAGU
GAACCAGUACCGUGAGGGAAAGGCGAAAAGAACCCCGGCGAGGGGAGUGAAAAAGAACCUGAA
ACCGUGUACGUACAAGCAGUGGGAGCACGCUUAGGCGUGUGACUGCGUACCUUUUGUAUAAUG
GGUCAGCGACUUAUAUUCUGUAGCAAGGUUAACCGAAUAGGGGAGCCGAAGGGAAACCGAGUC
UUAACUGGGCGUUAAGUUGCAGGGUAUAGACCCGAAACCCGGUGAUCUAGCCAUGGGCAGGUU
GAAGGUUGGGUAACACUAACUGGAGGACCGAACCGACUAAUGUUGAAAAAUUAGCGGAUGACU
UGUGGCUGGGGGUGAAAGGCCAAUCAAACCGGGAGAUAGCUGGUUCUCCCCGAAAGCUAUUUA
GGUAGCGCCUCGUGAAUUCAUCUCCGGGGGUAGAGCACUGUUUCGGCAAGGGGGUCAUCCCGA
CUUACCAACCCGAUGCAAACUGCGAAUACCGGAGAAUGUUAUCACGGGAGACACACGGCGGGU
GCUAACGUCCGUCGUGAAGAGGGAAACAACCCAGACCGCCAGCUAAGGUCCCAAAGUCAUGGU
UAAGUGGGAAACGAUGUGGGAAGGCCCAGACAGCCAGGAUGUUGGCUUAGAAGCAGCCAUCAU
UUAAAGAAAGCGUAAUAGCUCACUGGUCGAGUCGGCCUGCGCGGAAGAUGUAACGGGGCUAAA
CCAUGCACCGAAGCUGCGGCAGCGACGCUUAUGCGUUGUUGGGUAGGGGAGCGUUCUGUAAGC
CUGCGAAGGUGUGCUGUGAGGCAUGCUGGAGGUAUCAGAAGUGCGAAUGCUGACAUAAGUAAC
GAUAAAGCGGGUGAAAAGCCCGCUCGCCGGAAGACCAAGGGUUCCUGUCCAACGUUAAUCGGG
GCAGGGUGAGUCGACCCCUAAGGCGAGGCCGAAAGGCGUAGUCGAUGGGAAACAGGUUAAUAU
UCCUGUACUUGGUGUUACUGCGAAGGGGGGACGGAGAAGGCUAUGUUGGCCGGGCGACGGUUG
UCCCGGUUUAAGCGUGUAGGCUGGUUUUCCAGGCAAAUCCGGAAAAUCAAGGCUGAGGCGUGA
UGACGAGGCACUACGGUGCUGAAGCAACAAAUGCCCUGCUUCCAGGAAAAGCCUCUAAGCAUC
AGGUAACAUCAAAUCGUACCCCAAACCGACACAGGUGGUCAGGUAGAGAAUACCAAGGCGCUU
GAGAGAACUCGGGUGAAGGAACUAGGCAAAAUGGUGCCGUAACUUCGGGAGAAGGCACGCUGA
UAUGUAGGUGAGGUCCCUCGCGGAUGGAGCUGAAAUCAGUCGAAGAUACCAGCUGGCUGCAAC
UGUUUAUUAAAAACACAGCACUGUGCAAACACGAAAGUGGACGUAUACGGUGUGACGCCUGCC
CGGUGCCGGAAGGUUAAUUGAUGGGGUUAGCGCAAGCGAAGCUCUUGAUCGAAGCCCCGGUAA
ACGGCGGCCGUAACUAUAACGGUCCUAAGGUAGCGAAAUUCCUUGUCGGGUAAGUUCCGACCU
GCACGAAUGGCGUAAUGAUGGCCAGGCUGUCUCCACCCGAGACUCAGUGAAAUUGAACUCGCU
GUGAAGAUGCAGUGUACCCGCGGCAAGACGGAAAGACCCCGUGAACCUUUACUAUAGCUUGAC
ACUGAACAUUGAGCCUUGAUGUGUAGGAUAGGUGGGAGGCUUUGAAGUGUGGACGCCAGUCUG
CAUGGAGCCGAGCUUGAAAUACCACCCUUUAAUGUUUGAUGUUCUAACGUUGACCGGUAAUCC
GGGUUGCGGACAGUGUCUGGUGGGUAGUUUGACUGGGGCGGUCUCCUCCUAAAGAGUAACGGA
GGAGCACGAAGGUUGGCUAAUCCUGGUCGGACAUCAGGAGGUUAGUGCAAUGGCAUAAGCCAG
CUUGACUGCGAGCGUGACGGCGCGAGCAGGUGCGAAAGCAGGUCAUAGUGAUCCGGUGGUUCU
GAAUGGAAGGGCCAUCGCUCAACGGAUAAAAGGUACUCCGGGGAUAACAGGCUGAUACCGCCC
AAGAGUUCAUAUCGACGGCGGUGUUUGGCACCUCGAUGUCGGCUCAUCACAUCCUGGGGCUGA
AGUAGGUCCCAAGGGUAUGGCUGUUCGCCAUUUAAAGUGGUACGCGAGCUGGGUUUAGAACGU
CGUGAGACAGUUCGGUCCCUAUCUGCCGUGGGCGCUGGAGAACUGAGGGGGGCUGCUCCUAGU
ACGAGAGGACCGGAGUGGACGCAUCACUGGUGUUCGGGUUGUCAUGCCAAUGGCACUGCCCGG
UAGCUAAAUGCGGAAGAGAUAAGUGCUGAAAGCAUCUAAGCACGAAACUUGCCCCGAGAUGAG
UUCUCCCUGACCCUUUAAGGGUCCUGAAGGAACGUUGAAGACGACGACGUUGAUAGGCCGGGU
GUGUAAGGACUCACAACGGAGGGCGCUUACCACUUUGUGAUUCAUGACUGGGGUGAAGUCGUA
ACAAGGUAACCGUAGGGGAACCUGCGGUUGGAUCACUGUGGUA
Pair 11: Full Sequence (SEQ ID NO: 11)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
GUUAUAGUCUUGAGCUAACCGGUACUAAUGAACCGUGAGGCUUAACCGAGAGGUUAAGCGACU
AAGCGUACACGGUGGAUGCCCUGGCAGUCAGAGGCGAUGAAGGACGUGCUAAUCUGCGAUAAG
CGUCGGUAAGGUGAUAUGAACCGUUAUAACCGGCGAUUUCCGAAUGGGGAAACCCAGUGUGUU
UCGACACACUAUCAUUAACUGAAUCCAUAGGUUAAUGAGGCGAACCGGGGGAACUGAAACAUC
UAAGUACCCCGAGGAAAAGAAAUCAACCGAGAUUCCCCCAGUAGCGGCGAGCGAACGGGGAGC
AGCCCAGAGCCUGAAUCAGUGUGUGUGUUAGUGGAAGCGUCUGGAAAGGCGCGCGAUACAGGG
UGACAGCCCCGUACACAAAAAUGCACAUGCUGUGAGCUCGAUGAGUAGGGCGGGACACGUGGU
AUCCUGUCUGAAUAUGGGGGGACCAUCCUCCAAGGCUAAAUACUCCUGACUGACCGAUAGUGA
ACCAGUACCGUGAGGGAAAGGCGAAAAGAACCCCGGCGAGGGGAGUGAAAAAGAACCUGAAAC
CGUGUACGUACAAGCAGUGGGAGCACGCUUAGGCGUGUGACUGCGUACCUUUUGUAUAAUGGG
UCAGCGACUUAUAUUCUGUAGCAAGGUUAACCGAAUAGGGGAGCCGAAGGGAAACCGAGUCUU
AACUGGGCGUUAAGUUGCAGGGUAUAGACCCGAAACCCGGUGAUCUAGCCAUGGGCAGGUUGA
AGGUUGGGUAACACUAACUGGAGGACCGAACCGACUAAUGUUGAAAAAUUAGCGGAUGACUUG
UGGCUGGGGGUGAAAGGCCAAUCAAACCGGGAGAUAGCUGGUUCUCCCCGAAAGCUAUUUAGG
UAGCGCCUCGUGAAUUCAUCUCCGGGGGUAGAGCACUGUUUCGGCAAGGGGGUCAUCCCGACU
UACCAACCCGAUGCAAACUGCGAAUACCGGAGAAUGUUAUCACGGGAGACACACGGCGGGUGC
UAACGUCCGUCGUGAAGAGGGAAACAACCCAGACCGCCAGCUAAGGUCCCAAAGUCAUGGUUA
AGUGGGAAACGAUGUGGGAAGGCCCAGACAGCCAGGAUGUUGGCUUAGAAGCAGCCAUCAUUU
AAAGAAAGCGUAAUAGCUCACUGGUCGAGUCGGCCUGCGCGGAAGAUGUAACGGGGCUAAACC
AUGCACCGAAGCUGCGGCAGCGACGCUUAUGCGUUGUUGGGUAGGGGAGCGUUCUGUAAGCCU
GCGAAGGUGUGCUGUGAGGCAUGCUGGAGGUAUCAGAAGUGCGAAUGCUGACAUAAGUAACGA
UAAAGCGGGUGAAAAGCCCGCUCGCCGGAAGACCAAGGGUUCCUGUCCAACGUUAAUCGGGGC
AGGGUGAGUCGACCCCUAAGGCGAGGCCGAAAGGCGUAGUCGAUGGGAAACAGGUUAAUAUUC
CUGUACUUGGUGUUACUGCGAAGGGGGGACGGAGAAGGCUAUGUUGGCCGGGCGACGGUUGUC
CCGGUUUAAGCGUGUAGGCUGGUUUUCCAGGCAAAUCCGGAAAAUCAAGGCUGAGGCGUGAUG
ACGAGGCACUACGGUGCUGAAGCAACAAAUGCCCUGCUUCCAGGAAAAGCCUCUAAGCAUCAG
GUAACAUCAAAUCGUACCCCAAACCGACACAGGUGGUCAGGUAGAGAAUACCAAGGCGCUUGA
GAGAACUCGGGUGAAGGAACUAGGCAAAAUGGUGCCGUAACUUCGGGAGAAGGCACGCUGAUA
UGUAGGUGAGGUCCCUCGCGGAUGGAGCUGAAAUCAGUCGAAGAUACCAGCUGGCUGCAACUG
UUUAUUAAAAACACAGCACUGUGCAAACACGAAAGUGGACGUAUACGGUGUGACGCCUGCCCG
GUGCCGGAAGGUUAAUUGAUGGGGUUAGCGCAAGCGAAGCUCUUGAUCGAAGCCCCGGUAAAC
GGCGGCCGUAACUAUAACGGUCCUAAGGUAGCGAAAUUCCUUGUCGGGUAAGUUCCGACCUGC
ACGAAUGGCGUAAUGAUGGCCAGGCUGUCUCCACCCGAGACUCAGUGAAAUUGAACUCGCUGU
GAAGAUGCAGUGUACCCGCGGCAAGACGGAAAGACCCCGUGAACCUUUACUAUAGCUUGACAC
UGAACAUUGAGCCUUGAUGUGUAGGAUAGGUGGGAGGCUUUGAAGUGUGGACGCCAGUCUGCA
UGGAGCCGAGCUUGAAAUACCACCCUUUAAUGUUUGAUGUUCUAACGUUGACCGGUAAUCCGG
GUUGCGGACAGUGUCUGGUGGGUAGUUUGACUGGGGCGGUCUCCUCCUAAAGAGUAACGGAGG
AGCACGAAGGUUGGCUAAUCCUGGUCGGACAUCAGGAGGUUAGUGCAAUGGCAUAAGCCAGCU
UGACUGCGAGCGUGACGGCGCGAGCAGGUGCGAAAGCAGGUCAUAGUGAUCCGGUGGUUCUGA
AUGGAAGGGCCAUCGCUCAACGGAUAAAAGGUACUCCGGGGAUAACAGGCUGAUACCGCCCAA
GAGUUCAUAUCGACGGCGGUGUUUGGCACCUCGAUGUCGGCUCAUCACAUCCUGGGGCUGAAG
UAGGUCCCAAGGGUAUGGCUGUUCGCCAUUUAAAGUGGUACGCGAGCUGGGUUUAGAACGUCG
UGAGACAGUUCGGUCCCUAUCUGCCGUGGGCGCUGGAGAACUGAGGGGGGCUGCUCCUAGUAC
GAGAGGACCGGAGUGGACGCAUCACUGGUGUUCGGGUUGUCAUGCCAAUGGCACUGCCCGGUA
GCUAAAUGCGGAAGAGAUAAGUGCUGAAAGCAUCUAAGCACGAAACUUGCCCCGAGAUGAGUU
CUCCCUGACCCUUUAAGGGUCCUGAAGGAACGUUGAAGACGACGACGUUGAUAGGCCGGGUGU
GUAAGGACGACCUUCGGGAGGGCGCUUACCACUUUGUGAUUCAUGACUGGGGUGAAGUCGUAA
CAAGGUAACCGUAGGGGAACCUGCGGUUGGAUCACUGUGGUA
Pair 12: Full Sequence (SEQ ID NO: 12)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
AGUCAAUAAGUCUUGAGCUAACCGGUACUAAUGAACCGUGAGGCUUAACCGAGAGGUUAAGCG
ACUAAGCGUACACGGUGGAUGCCCUGGCAGUCAGAGGCGAUGAAGGACGUGCUAAUCUGCGAU
AAGCGUCGGUAAGGUGAUAUGAACCGUUAUAACCGGCGAUUUCCGAAUGGGGAAACCCAGUGU
GUUUCGACACACUAUCAUUAACUGAAUCCAUAGGUUAAUGAGGCGAACCGGGGGAACUGAAAC
AUCUAAGUACCCCGAGGAAAAGAAAUCAACCGAGAUUCCCCCAGUAGCGGCGAGCGAACGGGG
AGCAGCCCAGAGCCUGAAUCAGUGUGUGUGUUAGUGGAAGCGUCUGGAAAGGCGCGCGAUACA
GGGUGACAGCCCCGUACACAAAAAUGCACAUGCUGUGAGCUCGAUGAGUAGGGCGGGACACGU
GGUAUCCUGUCUGAAUAUGGGGGGACCAUCCUCCAAGGCUAAAUACUCCUGACUGACCGAUAG
UGAACCAGUACCGUGAGGGAAAGGCGAAAAGAACCCCGGCGAGGGGAGUGAAAAAGAACCUGA
AACCGUGUACGUACAAGCAGUGGGAGCACGCUUAGGCGUGUGACUGCGUACCUUUUGUAUAAU
GGGUCAGCGACUUAUAUUCUGUAGCAAGGUUAACCGAAUAGGGGAGCCGAAGGGAAACCGAGU
CUUAACUGGGCGUUAAGUUGCAGGGUAUAGACCCGAAACCCGGUGAUCUAGCCAUGGGCAGGU
UGAAGGUUGGGUAACACUAACUGGAGGACCGAACCGACUAAUGUUGAAAAAUUAGCGGAUGAC
UUGUGGCUGGGGGUGAAAGGCCAAUCAAACCGGGAGAUAGCUGGUUCUCCCCGAAAGCUAUUU
AGGUAGCGCCUCGUGAAUUCAUCUCCGGGGGUAGAGCACUGUUUCGGCAAGGGGGUCAUCCCG
ACUUACCAACCCGAUGCAAACUGCGAAUACCGGAGAAUGUUAUCACGGGAGACACACGGCGGG
UGCUAACGUCCGUCGUGAAGAGGGAAACAACCCAGACCGCCAGCUAAGGUCCCAAAGUCAUGG
UUAAGUGGGAAACGAUGUGGGAAGGCCCAGACAGCCAGGAUGUUGGCUUAGAAGCAGCCAUCA
UUUAAAGAAAGCGUAAUAGCUCACUGGUCGAGUCGGCCUGCGCGGAAGAUGUAACGGGGCUAA
ACCAUGCACCGAAGCUGCGGCAGCGACGCUUAUGCGUUGUUGGGUAGGGGAGCGUUCUGUAAG
CCUGCGAAGGUGUGCUGUGAGGCAUGCUGGAGGUAUCAGAAGUGCGAAUGCUGACAUAAGUAA
CGAUAAAGCGGGUGAAAAGCCCGCUCGCCGGAAGACCAAGGGUUCCUGUCCAACGUUAAUCGG
GGCAGGGUGAGUCGACCCCUAAGGCGAGGCCGAAAGGCGUAGUCGAUGGGAAACAGGUUAAUA
UUCCUGUACUUGGUGUUACUGCGAAGGGGGGACGGAGAAGGCUAUGUUGGCCGGGCGACGGUU
GUCCCGGUUUAAGCGUGUAGGCUGGUUUUCCAGGCAAAUCCGGAAAAUCAAGGCUGAGGCGUG
AUGACGAGGCACUACGGUGCUGAAGCAACAAAUGCCCUGCUUCCAGGAAAAGCCUCUAAGCAU
CAGGUAACAUCAAAUCGUACCCCAAACCGACACAGGUGGUCAGGUAGAGAAUACCAAGGCGCU
UGAGAGAACUCGGGUGAAGGAACUAGGCAAAAUGGUGCCGUAACUUCGGGAGAAGGCACGCUG
AUAUGUAGGUGAGGUCCCUCGCGGAUGGAGCUGAAAUCAGUCGAAGAUACCAGCUGGCUGCAA
CUGUUUAUUAAAAACACAGCACUGUGCAAACACGAAAGUGGACGUAUACGGUGUGACGCCUGC
CCGGUGCCGGAAGGUUAAUUGAUGGGGUUAGCGCAAGCGAAGCUCUUGAUCGAAGCCCCGGUA
AACGGCGGCCGUAACUAUAACGGUCCUAAGGUAGCGAAAUUCCUUGUCGGGUAAGUUCCGACC
UGCACGAAUGGCGUAAUGAUGGCCAGGCUGUCUCCACCCGAGACUCAGUGAAAUUGAACUCGC
UGUGAAGAUGCAGUGUACCCGCGGCAAGACGGAAAGACCCCGUGAACCUUUACUAUAGCUUGA
CACUGAACAUUGAGCCUUGAUGUGUAGGAUAGGUGGGAGGCUUUGAAGUGUGGACGCCAGUCU
GCAUGGAGCCGAGCUUGAAAUACCACCCUUUAAUGUUUGAUGUUCUAACGUUGACCCGUAAUC
CGGGUUGCGGACAGUGUCUGGUGGGUAGUUUGACUGGGGCGGUCUCCUCCUAAAGAGUAACGG
AGGAGCACGAAGGUUGGCUAAUCCUGGUCGGACAUCAGGAGGUUAGUGCAAUGGCAUAAGCCA
GCUUGACUGCGAGCGUGACGGCGCGAGCAGGUGCGAAAGCAGGUCAUAGUGAUCCGGUGGUUC
UGAAUGGAAGGGCCAUCGCUCAACGGAUAAAAGGUACUCCGGGGAUAACAGGCUGAUACCGCC
CAAGAGUUCAUAUCGACGGCGGUGUUUGGCACCUCGAUGUCGGCUCAUCACAUCCUGGGGCUG
AAGUAGGUCCCAAGGGUAUGGCUGUUCGCCAUUUAAAGUGGUACGCGAGCUGGGUUUAGAACG
UCGUGAGACAGUUCGGUCCCUAUCUGCCGUGGGCGCUGGAGAACUGAGGGGGGCUGCUCCUAG
UACGAGAGGACCGGAGUGGACGCAUCACUGGUGUUCGGGUUGUCAUGCCAAUGGCACUGCCCG
GUAGCUAAAUGCGGAAGAGAUAAGUGCUGAAAGCAUCUAAGCACGAAACUUGCCCCGAGAUGA
GUUCUCCCUGACCCUUUAAGGGUCCUGAAGGAACGUUGAAGACGACGACGUUGAUAGGCCGGG
UGUGUAAGGACACAUAAUGGGAGGGCGCUUACCACUUUGUGAUUCAUGACUGGGGUGAAGUCG
UAACAAGGUAACCGUAGGGGAACCUGCGGUUGGAUCACUGUGGUA
Pair 13: Full Sequence (SEQ ID NO: 13)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
CAUCAUGGGUCUUGAGCUAACCGGUACUAAUGAACCGUGAGGCUUAACCGAGAGGUUAAGCGA
CUAAGCGUACACGGUGGAUGCCCUGGCAGUCAGAGGCGAUGAAGGACGUGCUAAUCUGCGAUA
AGCGUCGGUAAGGUGAUAUGAACCGUUAUAACCGGCGAUUUCCGAAUGGGGAAACCCAGUGUG
UUUCGACACACUAUCAUUAACUGAAUCCAUAGGUUAAUGAGGCGAACCGGGGGAACUGAAACA
UCUAAGUACCCCGAGGAAAAGAAAUCAACCGAGAUUCCCCCAGUAGCGGCGAGCGAACGGGGA
GCAGCCCAGAGCCUGAAUCAGUGUGUGUGUUAGUGGAAGCGUCUGGAAAGGCGCGCGAUACAG
GGUGACAGCCCCGUACACAAAAAUGCACAUGCUGUGAGCUCGAUGAGUAGGGCGGGACACGUG
GUAUCCUGUCUGAAUAUGGGGGGACCAUCCUCCAAGGCUAAAUACUCCUGACUGACCGAUAGU
GAACCAGUACCGUGAGGGAAAGGCGAAAAGAACCCCGGCGAGGGGAGUGAAAAAGAACCUGAA
ACCGUGUACGUACAAGCAGUGGGAGCACGCUUAGGCGUGUGACUGCGUACCUUUUGUAUAAUG
GGUCAGCGACUUAUAUUCUGUAGCAAGGUUAACCGAAUAGGGGAGCCGAAGGGAAACCGAGUC
UUAACUGGGCGUUAAGUUGCAGGGUAUAGACCCGAAACCCGGUGAUCUAGCCAUGGGCAGGUU
GAAGGUUGGGUAACACUAACUGGAGGACCGAACCGACUAAUGUUGAAAAAUUAGCGGAUGACU
UGUGGCUGGGGGUGAAAGGCCAAUCAAACCGGGAGAUAGCUGGUUCUCCCCGAAAGCUAUUUA
GGUAGCGCCUCGUGAAUUCAUCUCCGGGGGUAGAGCACUGUUUCGGCAAGGGGGUCAUCCCGA
CUUACCAACCCGAUGCAAACUGCGAAUACCGGAGAAUGUUAUCACGGGAGACACACGGCGGGU
GCUAACGUCCGUCGUGAAGAGGGAAACAACCCAGACCGCCAGCUAAGGUCCCAAAGUCAUGGU
UAAGUGGGAAACGAUGUGGGAAGGCCCAGACAGCCAGGAUGUUGGCUUAGAAGCAGCCAUCAU
UUAAAGAAAGCGUAAUAGCUCACUGGUCGAGUCGGCCUGCGCGGAAGAUGUAACGGGGCUAAA
CCAUGCACCGAAGCUGCGGCAGCGACGCUUAUGCGUUGUUGGGUAGGGGAGCGUUCUGUAAGC
CUGCGAAGGUGUGCUGUGAGGCAUGCUGGAGGUAUCAGAAGUGCGAAUGCUGACAUAAGUAAC
GAUAAAGCGGGUGAAAAGCCCGCUCGCCGGAAGACCAAGGGUUCCUGUCCAACGUUAAUCGGG
GCAGGGUGAGUCGACCCCUAAGGCGAGGCCGAAAGGCGUAGUCGAUGGGAAACAGGUUAAUAU
UCCUGUACUUGGUGUUACUGCGAAGGGGGGACGGAGAAGGCUAUGUUGGCCGGGCGACGGUUG
UCCCGGUUUAAGCGUGUAGGCUGGUUUUCCAGGCAAAUCCGGAAAAUCAAGGCUGAGGCGUGA
UGACGAGGCACUACGGUGCUGAAGCAACAAAUGCCCUGCUUCCAGGAAAAGCCUCUAAGCAUC
AGGUAACAUCAAAUCGUACCCCAAACCGACACAGGUGGUCAGGUAGAGAAUACCAAGGCGCUU
GAGAGAACUCGGGUGAAGGAACUAGGCAAAAUGGUGCCGUAACUUCGGGAGAAGGCACGCUGA
UAUGUAGGUGAGGUCCCUCGCGGAUGGAGCUGAAAUCAGUCGAAGAUACCAGCUGGCUGCAAC
UGUUUAUUAAAAACACAGCACUGUGCAAACACGAAAGUGGACGUAUACGGUGUGACGCCUGCC
CGGUGCCGGAAGGUUAAUUGAUGGGGUUAGCGCAAGCGAAGCUCUUGAUCGAAGCCCCGGUAA
ACGGCGGCCGUAACUAUAACGGUCCUAAGGUAGCGAAAUUCCUUGUCGGGUAAGUUCCGACCU
GCACGAAUGGCGUAAUGAUGGCCAGGCUGUCUCCACCCGAGACUCAGUGAAAUUGAACUCGCU
GUGAAGAUGCAGUGUACCCGCGGCAAGACGGAAAGACCCCGUGAACCUUUACUAUAGCUUGAC
ACUGAACAUUGAGCCUUGAUGUGUAGGAUAGGUGGGAGGCUUUGAAGUGUGGACGCCAGUCUG
CAUGGAGCCGAGCUUGAAAUACCACCCUUUAAUGUUUGAUGUUCUAACGUUGACCGGUAAUCC
GGGUUGCGGACAGUGUCUGGUGGGUAGUUUGACUGGGGCGGUCUCCUCCUAAAGAGUAACGGA
GGAGCACGAAGGUUGGCUAAUCCUGGUCGGACAUCAGGAGGUUAGUGCAAUGGCAUAAGCCAG
CUUGACUGCGAGCGUGACGGCGCGAGCAGGUGCGAAAGCAGGUCAUAGUGAUCCGGUGGUUCU
GAAUGGAAGGGCCAUCGCUCAACGGAUAAAAGGUACUCCGGGGAUAACAGGCUGAUACCGCCC
AAGAGUUCAUAUCGACGGCGGUGUUUGGCACCUCGAUGUCGGCUCAUCACAUCCUGGGGCUGA
AGUAGGUCCCAAGGGUAUGGCUGUUCGCCAUUUAAAGUGGUACGCGAGCUGGGUUUAGAACGU
CGUGAGACAGUUCGGUCCCUAUCUGCCGUGGGCGCUGGAGAACUGAGGGGGGCUGCUCCUAGU
ACGAGAGGACCGGAGUGGACGCAUCACUGGUGUUCGGGUUGUCAUGCCAAUGGCACUGCCCGG
UAGCUAAAUGCGGAAGAGAUAAGUGCUGAAAGCAUCUAAGCACGAAACUUGCCCCGAGAUGAG
UUCUCCCUGACCCUUUAAGGGUCCUGAAGGAACGUUGAAGACGACGACGUUGAUAGGCCGGGU
GUGUAAGGACGACCUUCGGGAGGGCGCUUACCACUUUGUGAUUCAUGACUGGGGUGAAGUCGU
AACAAGGUAACCGUAGGGGAACCUGCGGUUGGAUCACUGUGGUA
Pair 14: Full Sequence (SEQ ID NO: 14)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
CAUCAUGGGUCUUGAGCUAACCGGUACUAAUGAACCGUGAGGCUUAACCGAGAGGUUAAGCGA
CUAAGCGUACACGGUGGAUGCCCUGGCAGUCAGAGGCGAUGAAGGACGUGCUAAUCUGCGAUA
AGCGUCGGUAAGGUGAUAUGAACCGUUAUAACCGGCGAUUUCCGAAUGGGGAAACCCAGUGUG
UUUCGACACACUAUCAUUAACUGAAUCCAUAGGUUAAUGAGGCGAACCGGGGGAACUGAAACA
UCUAAGUACCCCGAGGAAAAGAAAUCAACCGAGAUUCCCCCAGUAGCGGCGAGCGAACGGGGA
GCAGCCCAGAGCCUGAAUCAGUGUGUGUGUUAGUGGAAGCGUCUGGAAAGGCGCGCGAUACAG
GGUGACAGCCCCGUACACAAAAAUGCACAUGCUGUGAGCUCGAUGAGUAGGGCGGGACACGUG
GUAUCCUGUCUGAAUAUGGGGGGACCAUCCUCCAAGGCUAAAUACUCCUGACUGACCGAUAGU
GAACCAGUACCGUGAGGGAAAGGCGAAAAGAACCCCGGCGAGGGGAGUGAAAAAGAACCUGAA
ACCGUGUACGUACAAGCAGUGGGAGCACGCUUAGGCGUGUGACUGCGUACCUUUUGUAUAAUG
GGUCAGCGACUUAUAUUCUGUAGCAAGGUUAACCGAAUAGGGGAGCCGAAGGGAAACCGAGUC
UUAACUGGGCGUUAAGUUGCAGGGUAUAGACCCGAAACCCGGUGAUCUAGCCAUGGGCAGGUU
GAAGGUUGGGUAACACUAACUGGAGGACCGAACCGACUAAUGUUGAAAAAUUAGCGGAUGACU
UGUGGCUGGGGGUGAAAGGCCAAUCAAACCGGGAGAUAGCUGGUUCUCCCCGAAAGCUAUUUA
GGUAGCGCCUCGUGAAUUCAUCUCCGGGGGUAGAGCACUGUUUCGGCAAGGGGGUCAUCCCGA
CUUACCAACCCGAUGCAAACUGCGAAUACCGGAGAAUGUUAUCACGGGAGACACACGGCGGGU
GCUAACGUCCGUCGUGAAGAGGGAAACAACCCAGACCGCCAGCUAAGGUCCCAAAGUCAUGGU
UAAGUGGGAAACGAUGUGGGAAGGCCCAGACAGCCAGGAUGUUGGCUUAGAAGCAGCCAUCAU
UUAAAGAAAGCGUAAUAGCUCACUGGUCGAGUCGGCCUGCGCGGAAGAUGUAACGGGGCUAAA
CCAUGCACCGAAGCUGCGGCAGCGACGCUUAUGCGUUGUUGGGUAGGGGAGCGUUCUGUAAGC
CUGCGAAGGUGUGCUGUGAGGCAUGCUGGAGGUAUCAGAAGUGCGAAUGCUGACAUAAGUAAC
GAUAAAGCGGGUGAAAAGCCCGCUCGCCGGAAGACCAAGGGUUCCUGUCCAACGUUAAUCGGG
GCAGGGUGAGUCGACCCCUAAGGCGAGGCCGAAAGGCGUAGUCGAUGGGAAACAGGUUAAUAU
UCCUGUACUUGGUGUUACUGCGAAGGGGGGACGGAGAAGGCUAUGUUGGCCGGGCGACGGUUG
UCCCGGUUUAAGCGUGUAGGCUGGUUUUCCAGGCAAAUCCGGAAAAUCAAGGCUGAGGCGUGA
UGACGAGGCACUACGGUGCUGAAGCAACAAAUGCCCUGCUUCCAGGAAAAGCCUCUAAGCAUC
AGGUAACAUCAAAUCGUACCCCAAACCGACACAGGUGGUCAGGUAGAGAAUACCAAGGCGCUU
GAGAGAACUCGGGUGAAGGAACUAGGCAAAAUGGUGCCGUAACUUCGGGAGAAGGCACGCUGA
UAUGUAGGUGAGGUCCCUCGCGGAUGGAGCUGAAAUCAGUCGAAGAUACCAGCUGGCUGCAAC
UGUUUAUUAAAAACACAGCACUGUGCAAACACGAAAGUGGACGUAUACGGUGUGACGCCUGCC
CGGUGCCGGAAGGUUAAUUGAUGGGGUUAGCGCAAGCGAAGCUCUUGAUCGAAGCCCCGGUAA
ACGGCGGCCGUAACUAUAACGGUCCUAAGGUAGCGAAAUUCCUUGUCGGGUAAGUUCCGACCU
GCACGAAUGGCGUAAUGAUGGCCAGGCUGUCUCCACCCGAGACUCAGUGAAAUUGAACUCGCU
GUGAAGAUGCAGUGUACCCGCGGCAAGACGGAAAGACCCCGUGAACCUUUACUAUAGCUUGAC
ACUGAACAUUGAGCCUUGAUGUGUAGGAUAGGUGGGAGGCUUUGAAGUGUGGACGCCAGUCUG
CAUGGAGCCGAGCUUGAAAUACCACCCUUUAAUGUUUGAUGUUCUAACGUUGACCGGUAAUCC
GGGUUGCGGACAGUGUCUGGUGGGUAGUUUGACUGGGGCGGUCUCCUCCUAAAGAGUAACGGA
GGAGCACGAAGGUUGGCUAAUCCUGGUCGGACAUCAGGAGGUUAGUGCAAUGGCAUAAGCCAG
CUUGACUGCGAGCGUGACGGCGCGAGCAGGUGCGAAAGCAGGUCAUAGUGAUCCGGUGGUUCU
GAAUGGAAGGGCCAUCGCUCAACGGAUAAAAGGUACUCCGGGGAUAACAGGCUGAUACCGCCC
AAGAGUUCAUAUCGACGGCGGUGUUUGGCACCUCGAUGUCGGCUCAUCACAUCCUGGGGCUGA
AGUAGGUCCCAAGGGUAUGGCUGUUCGCCAUUUAAAGUGGUACGCGAGCUGGGUUUAGAACGU
CGUGAGACAGUUCGGUCCCUAUCUGCCGUGGGCGCUGGAGAACUGAGGGGGGCUGCUCCUAGU
ACGAGAGGACCGGAGUGGACGCAUCACUGGUGUUCGGGUUGUCAUGCCAAUGGCACUGCCCGG
UAGCUAAAUGCGGAAGAGAUAAGUGCUGAAAGCAUCUAAGCACGAAACUUGCCCCGAGAUGAG
UUCUCCCUGACCCUUUAAGGGUCCUGAAGGAACGUUGAAGACGACGACGUUGAUAGGCCGGGU
GUGUAAGGACAUCCCAGGGGAGGGCGCUUACCACUUUGUGAUUCAUGACUGGGGUGAAGUCGU
AACAAGGUAACCGUAGGGGAACCUGCGGUUGGAUCACUGUGGUA
Pair 15: Full Sequence (SEQ ID NO: 15)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
GUUAUAGUCUUGAGCUAACCGGUACUAAUGAACCGUGAGGCUUAACCGAGAGGUUAAGCGACU
AAGCGUACACGGUGGAUGCCCUGGCAGUCAGAGGCGAUGAAGGACGUGCUAAUCUGCGAUAAG
CGUCGGUAAGGUGAUAUGAACCGUUAUAACCGGCGAUUUCCGAAUGGGGAAACCCAGUGUGUU
UCGACACACUAUCAUUAACUGAAUCCAUAGGUUAAUGAGGCGAACCGGGGGAACUGAAACAUC
UAAGUACCCCGAGGAAAAGAAAUCAACCGAGAUUCCCCCAGUAGCGGCGAGCGAACGGGGAGC
AGCCCAGAGCCUGAAUCAGUGUGUGUGUUAGUGGAAGCGUCUGGAAAGGCGCGCGAUACAGGG
UGACAGCCCCGUACACAAAAAUGCACAUGCUGUGAGCUCGAUGAGUAGGGCGGGACACGUGGU
AUCCUGUCUGAAUAUGGGGGGACCAUCCUCCAAGGCUAAAUACUCCUGACUGACCGAUAGUGA
ACCAGUACCGUGAGGGAAAGGCGAAAAGAACCCCGGCGAGGGGAGUGAAAAAGAACCUGAAAC
CGUGUACGUACAAGCAGUGGGAGCACGCUUAGGCGUGUGACUGCGUACCUUUUGUAUAAUGGG
UCAGCGACUUAUAUUCUGUAGCAAGGUUAACCGAAUAGGGGAGCCGAAGGGAAACCGAGUCUU
AACUGGGCGUUAAGUUGCAGGGUAUAGACCCGAAACCCGGUGAUCUAGCCAUGGGCAGGUUGA
AGGUUGGGUAACACUAACUGGAGGACCGAACCGACUAAUGUUGAAAAAUUAGCGGAUGACUUG
UGGCUGGGGGUGAAAGGCCAAUCAAACCGGGAGAUAGCUGGUUCUCCCCGAAAGCUAUUUAGG
UAGCGCCUCGUGAAUUCAUCUCCGGGGGUAGAGCACUGUUUCGGCAAGGGGGUCAUCCCGACU
UACCAACCCGAUGCAAACUGCGAAUACCGGAGAAUGUUAUCACGGGAGACACACGGCGGGUGC
UAACGUCCGUCGUGAAGAGGGAAACAACCCAGACCGCCAGCUAAGGUCCCAAAGUCAUGGUUA
AGUGGGAAACGAUGUGGGAAGGCCCAGACAGCCAGGAUGUUGGCUUAGAAGCAGCCAUCAUUU
AAAGAAAGCGUAAUAGCUCACUGGUCGAGUCGGCCUGCGCGGAAGAUGUAACGGGGCUAAACC
AUGCACCGAAGCUGCGGCAGCGACGCUUAUGCGUUGUUGGGUAGGGGAGCGUUCUGUAAGCCU
GCGAAGGUGUGCUGUGAGGCAUGCUGGAGGUAUCAGAAGUGCGAAUGCUGACAUAAGUAACGA
UAAAGCGGGUGAAAAGCCCGCUCGCCGGAAGACCAAGGGUUCCUGUCCAACGUUAAUCGGGGC
AGGGUGAGUCGACCCCUAAGGCGAGGCCGAAAGGCGUAGUCGAUGGGAAACAGGUUAAUAUUC
CUGUACUUGGUGUUACUGCGAAGGGGGGACGGAGAAGGCUAUGUUGGCCGGGCGACGGUUGUC
CCGGUUUAAGCGUGUAGGCUGGUUUUCCAGGCAAAUCCGGAAAAUCAAGGCUGAGGCGUGAUG
ACGAGGCACUACGGUGCUGAAGCAACAAAUGCCCUGCUUCCAGGAAAAGCCUCUAAGCAUCAG
GUAACAUCAAAUCGUACCCCAAACCGACACAGGUGGUCAGGUAGAGAAUACCAAGGCGCUUGA
GAGAACUCGGGUGAAGGAACUAGGCAAAAUGGUGCCGUAACUUCGGGAGAAGGCACGCUGAUA
UGUAGGUGAGGUCCCUCGCGGAUGGAGCUGAAAUCAGUCGAAGAUACCAGCUGGCUGCAACUG
UUUAUUAAAAACACAGCACUGUGCAAACACGAAAGUGGACGUAUACGGUGUGACGCCUGCCCG
GUGCCGGAAGGUUAAUUGAUGGGGUUAGCGCAAGCGAAGCUCUUGAUCGAAGCCCCGGUAAAC
GGCGGCCGUAACUAUAACGGUCCUAAGGUAGCGAAAUUCCUUGUCGGGUAAGUUCCGACCUGC
ACGAAUGGCGUAAUGAUGGCCAGGCUGUCUCCACCCGAGACUCAGUGAAAUUGAACUCGCUGU
GAAGAUGCAGUGUACCCGCGGCAAGACGGAAAGACCCCGUGAACCUUUACUAUAGCUUGACAC
UGAACAUUGAGCCUUGAUGUGUAGGAUAGGUGGGAGGCUUUGAAGUGUGGACGCCAGUCUGCA
UGGAGCCGAGCUUGAAAUACCACCCUUUAAUGUUUGAUGUUCUAACGUUGACCGGUAAUCCGG
GUUGCGGACAGUGUCUGGUGGGUAGUUUGACUGGGGCGGUCUCCUCCUAAAGAGUAACGGAGG
AGCACGAAGGUUGGCUAAUCCUGGUCGGACAUCAGGAGGUUAGUGCAAUGGCAUAAGCCAGCU
UGACUGCGAGCGUGACGGCGCGAGCAGGUGCGAAAGCAGGUCAUAGUGAUCCGGUGGUUCUGA
AUGGAAGGGCCAUCGCUCAACGGAUAAAAGGUACUCCGGGGAUAACAGGCUGAUACCGCCCAA
GAGUUCAUAUCGACGGCGGUGUUUGGCACCUCGAUGUCGGCUCAUCACAUCCUGGGGCUGAAG
UAGGUCCCAAGGGUAUGGCUGUUCGCCAUUUAAAGUGGUACGCGAGCUGGGUUUAGAACGUCG
UGAGACAGUUCGGUCCCUAUCUGCCGUGGGCGCUGGAGAACUGAGGGGGGCUGCUCCUAGUAC
GAGAGGACCGGAGUGGACGCAUCACUGGUGUUCGGGUUGUCAUGCCAAUGGCACUGCCCGGUA
GCUAAAUGCGGAAGAGAUAAGUGCUGAAAGCAUCUAAGCACGAAACUUGCCCCGAGAUGAGUU
CUCCCUGACCCUUUAAGGGUCCUGAAGGAACGUUGAAGACGACGACGUUGAUAGGCCGGGUGU
GUAAGGACACAUAAUGGGAGGGCGCUUACCACUUUGUGAUUCAUGACUGGGGUGAAGUCGUAA
CAAGGUAACCGUAGGGGAACCUGCGGUUGGAUCACUGUGGUA
Pair 16: Full Sequence (SEQ ID NO: 16)
AAAUUGAAGAGUUUGAUCAUGGCUCAGAUUGAACGCUGGCGGCAGGCCUAACACAUGCAAGUC
GAACGGUAACAGGAAGAAGCUUGCUUCUUUGCUGACGAGUGGCGGACGGGUGAGUAAUGUCUG
GGAAACUGCCUGAUGGAGGGGGAUAACUACUGGAAACGGUAGCUAAUACCGCAUAACGUCGCA
AGACCAAAGAGGGGGACCUUCGGGCCUCUUGCCAUCGGAUGUGCCCAGAUGGGAUUAGCUAGU
AGGUGGGGUAACGGCUCACCUAGGCGACGAUCCCUAGCUGGUCUGAGAGGAUGACCAGCCACA
CUGGAACUGAGACACGGUCCAGACUCCUACGGGAGGCAGCAGUGGGGAAUAUUGCACAAUGGG
CGCAAGCCUGAUGCAGCCAUGCCGCGUGUAUGAAGAAGGCCUUCGGGUUGUAAAGUACUUUCA
GCGGGGAGGAAGGGAGUAAAGUUAAUACCUUUGCUCAUUGACGUUACCCGCAGAAGAAGCACC
GGCUAACUCCGUGCCAGCAGCCGCGGUAAUACGGAGGGUGCAAGCGUUAAUCGGAAUUACUGG
GCGUAAAGCGCACGCAGGCGGUUUGUUAAGUCAGAUGUGAAAUCCCCGGGCUCAACCUGGGAA
CUGCAUCUGAUACUGGCAAGCUUGAGUCUCGUAGAGGGGGGUAGAAUUCCAGGUGUAGCGGUG
AAAUGCGUAGAGAUCUGGAGGAAUACCGGUGGCGAAGGCGGCCCCCUGGACGAAGACUGACGC
UCAGGUGCGAAAGCGUGGGGAGCAAACAGGAUUAGAUACCCUGGUAGUCCACGCCGUAAACGA
UGUCGACUUGGAGGUUGUGCCCUUGAGGCGUGGCUUCCGGAGCUAACGCGUUAAGUCGACCGC
CUGGGGAGUACGGCCGCAAGGUUAAAACUCAAAUGAAUUGACGGGGGCCCGCACAAGCGGUGG
AGCAUGUGGUUUAAUUCGAUGCAACGCGAAGAACCUUACCUGGUCUUGACAUCCACGGAAGUU
UUCAGAGAUGAGAAUGUGCCUUCGGGAACCGUGAGACAGGUGCUGCAUGGCUGUCGUCAGCUC
GUGUUGUGAAAUGUUGGGUUAAGUCCCGCAACGAGCGCAACCCUUAUCCUUUGUUGCCAGCGG
UCCGGCCGGGAACUCAAAGGAGACUGCCAGUGAUAAACUGGAGGAAGGUGGGGAUGACGUCAA
GUCAUCAUGGCCCUUACGACCAGGGCUACACACGUGCUACAAUGGCGCAUACAAAGAGAAGCG
ACCUCGCGAGAGCAAGCGGACCUCAUAAAGUGCGUCGUAGUCCGGAUUGGAGUCUGCAACUCG
ACUCCAUGAAGUCGGAAUCGCUAGUAAUCGUGGAUCAGAAUGCCACGGUGAAUACGUUCCCGG
GCCUUGUACACACCGCCCGUCACACCAUGGGAGUGGGUUGCAAAAGAAGUAGGUAGCUUAACC
AUAUAAUGUCUUGAGCUAACCGGUACUAAUGAACCGUGAGGCUUAACCGAGAGGUUAAGCGAC
UAAGCGUACACGGUGGAUGCCCUGGCAGUCAGAGGCGAUGAAGGACGUGCUAAUCUGCGAUAA
GCGUCGGUAAGGUGAUAUGAACCGUUAUAACCGGCGAUUUCCGAAUGGGGAAACCCAGUGUGU
UUCGACACACUAUCAUUAACUGAAUCCAUAGGUUAAUGAGGCGAACCGGGGGAACUGAAACAU
CUAAGUACCCCGAGGAAAAGAAAUCAACCGAGAUUCCCCCAGUAGCGGCGAGCGAACGGGGAG
CAGCCCAGAGCCUGAAUCAGUGUGUGUGUUAGUGGAAGCGUCUGGAAAGGCGCGCGAUACAGG
GUGACAGCCCCGUACACAAAAAUGCACAUGCUGUGAGCUCGAUGAGUAGGGCGGGACACGUGG
UAUCCUGUCUGAAUAUGGGGGGACCAUCCUCCAAGGCUAAAUACUCCUGACUGACCGAUAGUG
AACCAGUACCGUGAGGGAAAGGCGAAAAGAACCCCGGCGAGGGGAGUGAAAAAGAACCUGAAA
CCGUGUACGUACAAGCAGUGGGAGCACGCUUAGGCGUGUGACUGCGUACCUUUUGUAUAAUGG
GUCAGCGACUUAUAUUCUGUAGCAAGGUUAACCGAAUAGGGGAGCCGAAGGGAAACCGAGUCU
UAACUGGGCGUUAAGUUGCAGGGUAUAGACCCGAAACCCGGUGAUCUAGCCAUGGGCAGGUUG
AAGGUUGGGUAACACUAACUGGAGGACCGAACCGACUAAUGUUGAAAAAUUAGCGGAUGACUU
GUGGCUGGGGGUGAAAGGCCAAUCAAACCGGGAGAUAGCUGGUUCUCCCCGAAAGCUAUUUAG
GUAGCGCCUCGUGAAUUCAUCUCCGGGGGUAGAGCACUGUUUCGGCAAGGGGGUCAUCCCGAC
UUACCAACCCGAUGCAAACUGCGAAUACCGGAGAAUGUUAUCACGGGAGACACACGGCGGGUG
CUAACGUCCGUCGUGAAGAGGGAAACAACCCAGACCGCCAGCUAAGGUCCCAAAGUCAUGGUU
AAGUGGGAAACGAUGUGGGAAGGCCCAGACAGCCAGGAUGUUGGCUUAGAAGCAGCCAUCAUU
UAAAGAAAGCGUAAUAGCUCACUGGUCGAGUCGGCCUGCGCGGAAGAUGUAACGGGGCUAAAC
CAUGCACCGAAGCUGCGGCAGCGACGCUUAUGCGUUGUUGGGUAGGGGAGCGUUCUGUAAGCC
UGCGAAGGUGUGCUGUGAGGCAUGCUGGAGGUAUCAGAAGUGCGAAUGCUGACAUAAGUAACG
AUAAAGCGGGUGAAAAGCCCGCUCGCCGGAAGACCAAGGGUUCCUGUCCAACGUUAAUCGGGG
CAGGGUGAGUCGACCCCUAAGGCGAGGCCGAAAGGCGUAGUCGAUGGGAAACAGGUUAAUAUU
CCUGUACUUGGUGUUACUGCGAAGGGGGGACGGAGAAGGCUAUGUUGGCCGGGCGACGGUUGU
CCCGGUUUAAGCGUGUAGGCUGGUUUUCCAGGCAAAUCCGGAAAAUCAAGGCUGAGGCGUGAU
GACGAGGCACUACGGUGCUGAAGCAACAAAUGCCCUGCUUCCAGGAAAAGCCUCUAAGCAUCA
GGUAACAUCAAAUCGUACCCCAAACCGACACAGGUGGUCAGGUAGAGAAUACCAAGGCGCUUG
AGAGAACUCGGGUGAAGGAACUAGGCAAAAUGGUGCCGUAACUUCGGGAGAAGGCACGCUGAU
AUGUAGGUGAGGUCCCUCGCGGAUGGAGCUGAAAUCAGUCGAAGAUACCAGCUGGCUGCAACU
GUUUAUUAAAAACACAGCACUGUGCAAACACGAAAGUGGACGUAUACGGUGUGACGCCUGCCC
GGUGCCGGAAGGUUAAUUGAUGGGGUUAGCGCAAGCGAAGCUCUUGAUCGAAGCCCCGGUAAA
CGGCGGCCGUAACUAUAACGGUCCUAAGGUAGCGAAAUUCCUUGUCGGGUAAGUUCCGACCUG
CACGAAUGGCGUAAUGAUGGCCAGGCUGUCUCCACCCGAGACUCAGUGAAAUUGAACUCGCUG
UGAAGAUGCAGUGUACCCGCGGCAAGACGGAAAGACCCCGUGAACCUUUACUAUAGCUUGACA
CUGAACAUUGAGCCUUGAUGUGUAGGAUAGGUGGGAGGCUUUGAAGUGUGGACGCCAGUCUGC
AUGGAGCCGAGCUUGAAAUACCACCCUUUAAUGUUUGAUGUUCUAACGUUGACCCGUAAUCCG
GGUUGCGGACAGUGUCUGGUGGGUAGUUUGACUGGGGCGGUCUCCUCCUAAAGAGUAACGGAG
GAGCACGAAGGUUGGCUAAUCCUGGUCGGACAUCAGGAGGUUAGUGCAAUGGCAUAAGCCAGC
UUGACUGCGAGCGUGACGGCGCGAGCAGGUGCGAAAGCAGGUCAUAGUGAUCCGGUGGUUCUG
AAUGGAAGGGCCAUCGCUCAACGGAUAAAAGGUACUCCGGGGAUAACAGGCUGAUACCGCCCA
AGAGUUCAUAUCGACGGCGGUGUUUGGCACCUCGAUGUCGGCUCAUCACAUCCUGGGGCUGAA
GUAGGUCCCAAGGGUAUGGCUGUUCGCCAUUUAAAGUGGUACGCGAGCUGGGUUUAGAACGUC
GUGAGACAGUUCGGUCCCUAUCUGCCGUGGGCGCUGGAGAACUGAGGGGGGCUGCUCCUAGUA
CGAGAGGACCGGAGUGGACGCAUCACUGGUGUUCGGGUUGUCAUGCCAAUGGCACUGCCCGGU
AGCUAAAUGCGGAAGAGAUAAGUGCUGAAAGCAUCUAAGCACGAAACUUGCCCCGAGAUGAGU
UCUCCCUGACCCUUUAAGGGUCCUGAAGGAACGUUGAAGACGACGACGUUGAUAGGCCGGGUG
UGUAAGGACACAUAAUGGGAGGGCGCUUACCACUUUGUGAUUCAUGACUGGGGUGAAGUCGUA
ACAAGGUAACCGUAGGGGAACCUGCGGUUGGAUCACUGUGGUA

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

2. The engineered ribosome of claim 1, wherein the T1 polynucleotide domain comprises 5′-GUUAUA-3′ and the T2 polynucleotide domain comprises 5′-UCACAAG-3′ (Pair 1).
3. The engineered ribosome of claim 1, wherein the T1 polynucleotide domain comprises 5′-AGUCAAUAA-3′ and T2 polynucleotide comprises 5′-GACCUUCG-3′ (Pair 2).
4. The engineered ribosome of claim 1 comprising SEQ ID NO: 1, or any one SEQ ID NOs 1-16.
5. A polynucleotide, the polynucleotide encoding the rRNA of the engineered ribosome of claim 1.
6. The polynucleotide of claim 5, wherein the polynucleotide is in a vector.
7. The polynucleotide of claim 6, wherein the polynucleotide further comprises a gene to be expressed by the engineered ribosome.
8. The polynucleotide of claim 7, wherein the engineered ribosome comprises a modified anti-Shine-Dalgarno sequence and the gene comprises a complementary Shine-Dalgarno sequence to the engineered ribosome.
9. The polynucleotide of claim 8 wherein the gene comprises one or more codons, wherein at least one of the one or more codons comprises a non-canonical codon or an unnatural codon.
10. The polynucleotide of claim 9, wherein the non-canonical codon or the unnatural codon codes for a non-canonical amino acid, or a non-amino acid monomer.
11. A method for preparing an engineered ribosome, the method comprising expressing the polynucleotide of claim 5.
12. A cell, the cell comprising (i) the polynucleotide of claim 5, (ii) the engineered ribosome of claim 1, or both (i) and (ii).
13. A cell, the cell comprising a first protein translation mechanism and a second protein translation mechanism;

What is claimed:

1. An engineered ribosome, the engineered ribosome comprising:
a) a small subunit comprising a 16S rRNA polynucleotide sequence or variant thereof,
b) a large subunit comprising a 23S rRNA polynucleotide sequence or variant thereof, and
c) a linking moiety comprising
a T1 polynucleotide domain and a T2 polynucleotide domain, wherein the linking moiety links the 16S RNA and the 23S rRNA, thereby linking the large and small ribosomal subunits;
wherein the linking moiety covalently bonds helix 101 of the 23S rRNA large subunit to helix 44 of the 16s rRNA of the small subunit; and
wherein the T1 domain and the T2 domain are paired, and the T1 and T2 domains comprise one of Pairs 1-16 as shown in the table below:

| PAIR | T1 (5′-3′) | T2 (5′-3′) |
|---|---|---|
| 1 | GUUAUA | UCACAAC |
| 2 | AGUCAAUAA | GACCUUCG |
| 3 | AUAUAAU | AUCCCAGG |
| 4 | AGUCAAUAA | UCACAAC |
| 5 | AUAUAAU | GACCUUCG |
| 6 | AUAUAAU | UCACAAC |
| 7 | GUUAUA | AUCCCAGG |
| 8 | CAUCAUGG | ACAUAAUG |
| 9 | AGUCAAUAA | AUCCCAGG |
| 10 | CAUCAUGG | UCACAAC |
| 11 | GUUAUA | GACCUUCG |
| 12 | AGUCAAUAA | ACAUAAUG |
| 13 | CAUCAUGG | GACCUUCG |
| 14 | CAUCAUGG | AUCCCAGG |
| 15 | GUUAUA | ACAUAAUG |
| 16 | AUAUAAU | ACAUAAUG |

wherein the first protein translation mechanism comprises a ribosome, wherein the ribosome lacks a linking moiety between the large subunit and the small subunit; and
wherein the second protein translation mechanism comprises the engineered ribosome of claim 1.

14. The cell of claim 13, wherein the cell comprises a bacterial cell.

15. The cell of claim 14, wherein the bacterial cell comprises an Escherichia coli cell.

16. A method for preparing a sequence-defined polymer, the method comprising:
(a) providing the engineered ribosome of claim 1; and
(b) providing an mRNA or DNA template encoding the sequence-defined polymer.

17. The method of claim 16, wherein the sequence-defined polymer is prepared in vitro.

18. The method of claim 17, the method further comprising providing a ribosome-depleted cellular extract or purified translation system.

19. The method of claim 16, wherein the sequence defined polymer is prepared in vivo.

20. The method of claim 16, wherein the sequence defined polymer is prepared in the cell of claim 13.

21. The method of claim 16, wherein the mRNA or DNA encodes a modified Shine-Dalgarno sequence and the engineered ribosome comprises an anti-Shine-Dalgarno sequence complementary to the modified Shine-Dalgarno sequence.

22. The method of claim 16, comprising flexizymes, wherein the mRNA or DNA template encoding the sequence defined polymer comprises one or more codons, wherein at least one of the one or more codons encodes a non-canonical amino acid, or a non-amino acid monomer.

23. A method for the directed evolution of a target nucleic acid sequence,
wherein the target nucleic acid sequence comprises at least two regions of interest, wherein the regions of interest are separated by an intervening sequence of at least 300 nucleotides in length, the method comprising:
(a) generating a library of test nucleic acid sequences, wherein each test nucleic acid sequence has a different nucleotide sequence for at least one of the regions of interest;
(b) screening the library for functional test nucleic acid sequences;
(c) sequencing the functional test nucleic acid sequences, wherein sequencing comprises:
(i) performing a first polymerase chain reaction (PCR), wherein the first PCR provides a first PCR product comprising the at least two regions of interest but does not include at least a portion of the intervening sequence;
(ii) performing a ligation reaction, wherein the ligation reaction provides a first ligation product comprising the two regions of interest, wherein after the ligation reaction, the two regions of interest are positioned less than 300 nucleotides apart;
(iii) performing a second PCR, wherein the second PCR provides a second PCR product comprising the two regions of interest;
(iv) sequencing the second PCR product comprising the two regions of interest.

24. The method of claim 23, wherein the sequencing comprises next generation sequencing.


SEQUENCE LISTING (113 sequences)

SEQ ID NO: 1 - RNA, 4451 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccguuauagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacucacaacggagggcgcuuaccacuuugugauucaugacuggggugaagucguaacaagguaaccguaggggaaccugcgguuggaucacuguggua

SEQ ID NO: 2 - RNA, 4455 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccagucaauaagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacgaccuucgggagggcgcuuaccacuuugugauucaugacuggggugaagucguaacaagguaaccguaggggaaccugcgguuggaucacuguggua

SEQ ID NO: 3 - RNA, 4453 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccauauaaugucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacaucccaggggagggcgcuuaccacuuugugauucaugacuggggugaagucguaacaagguaaccguaggggaaccugcgguuggaucacuguggua

SEQ ID NO: 4 - RNA, 4454 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccagucaauaagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacucacaacggagggcgcuuaccacuuugugauucaugacuggggugaagucguaacaagguaaccguaggggaaccugcgguuggaucacuguggua

SEQ ID NO: 5 - RNA, 4453 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccauauaaugucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacgaccuucgggagggcgcuuaccacuuugugauucaugacuggggugaagucguaacaagguaaccguaggggaaccugcgguuggaucacuguggua

SEQ ID NO: 6 - RNA, 4452 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccauauaaugucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacucacaacggagggcgcuuaccacuuugugauucaugacuggggugaagucguaacaagguaaccguaggggaaccugcgguuggaucacuguggua

SEQ ID NO: 7 - RNA, 4452 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccguuauagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacaucccaggggagggcgcuuaccacuuugugauucaugacuggggugaagucguaacaagguaaccguaggggaaccugcgguuggaucacuguggua

SEQ ID NO: 8 - RNA, 4454 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaacccaucaugggucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacacauaaugggagggcgcuuaccacuuugugauucaugacuggggugaagucguaacaagguaaccguaggggaaccugcgguuggaucacuguggua

SEQ ID NO: 9 - RNA, 4455 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccagucaauaagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacaucccaggggagggcgcuuaccacuuugugauucaugacuggggugaagucguaacaagguaaccguaggggaaccugcgguuggaucacuguggua

SEQ ID NO: 10 - RNA, 4455 nt, Artificial Sequence; identical to SEQ ID NO: 9
  Synthetic- modified ribosome RNA

SEQ ID NO: 11 - RNA, 4452 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccguuauagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacgaccuucgggagggcgcuuaccacuuugugauucaugacuggggugaagucguaacaagguaaccguaggggaaccugcgguuggaucacuguggua

SEQ ID NO: 12 - RNA, 4455 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccagucaauaagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacacauaaugggagggcgcuuaccacuuugugauucaugacuggggugaagucguaacaagguaaccguaggggaaccugcgguuggaucacuguggua

SEQ ID NO: 13 - RNA, 4454 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaacccaucaugggucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacgaccuucgggagggcgcuuaccacuuugugauucaugacuggggugaagucguaacaagguaaccguaggggaaccugcgguuggaucacuguggua

SEQ ID NO: 14 - RNA, 4454 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaacccaucaugggucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacaucccaggggagggcgcuuaccacuuugugauucaugacuggggugaagucguaacaagguaaccguaggggaaccugcgguuggaucacuguggua

SEQ ID NO: 15 - RNA, 4452 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccguuauagucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacacauaaugggagggcgcuuaccacuuugugauucaugacuggggugaagucguaacaagguaaccguaggggaaccugcgguuggaucacuguggua

SEQ ID NO: 16 - RNA, 4453 nt, Artificial Sequence
  Synthetic- modified ribosome RNA
aaauugaagaguuugaucauggcucagauugaacgcuggcggcaggccuaacacaugcaagucgaacgguaacaggaagaagcuugcuucuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccauauaaugucuugagcuaaccgguacuaaugaaccgugaggcuuaaccgagagguuaagcgacuaagcguacacgguggaugcccuggcagucagaggcgaugaaggacgugcuaaucugcgauaagcgucgguaaggugauaugaaccguuauaaccggcgauuuccgaauggggaaacccaguguguuucgacacacuaucauuaacugaauccauagguuaaugaggcgaaccgggggaacugaaacaucuaaguaccccgaggaaaagaaaucaaccgagauucccccaguagcggcgagcgaacggggagcagcccagagccugaaucaguguguguguuaguggaagcgucuggaaaggcgcgcgauacagggugacagccccguacacaaaaaugcacaugcugugagcucgaugaguagggcgggacacgugguauccugucugaauauggggggaccauccuccaaggcuaaauacuccugacugaccgauagugaaccaguaccgugagggaaaggcgaaaagaaccccggcgaggggagugaaaaagaaccugaaaccguguacguacaagcagugggagcacgcuuaggcgugugacugcguaccuuuuguauaaugggucagcgacuuauauucuguagcaagguuaaccgaauaggggagccgaagggaaaccgagucuuaacugggcguuaaguugcaggguauagacccgaaacccggugaucuagccaugggcagguugaagguuggguaacacuaacuggaggaccgaaccgacuaauguugaaaaauuagcggaugacuuguggcugggggugaaaggccaaucaaaccgggagauagcugguucuccccgaaagcuauuuagguagcgccucgugaauucaucuccggggguagagcacuguuucggcaagggggucaucccgacuuaccaacccgaugcaaacugcgaauaccggagaauguuaucacgggagacacacggcgggugcuaacguccgucgugaagagggaaacaacccagaccgccagcuaaggucccaaagucaugguuaagugggaaacgaugugggaaggcccagacagccaggauguuggcuuagaagcagccaucauuuaaagaaagcguaauagcucacuggucgagucggccugcgcggaagauguaacggggcuaaaccaugcaccgaagcugcggcagcgacgcuuaugcguuguuggguaggggagcguucuguaagccugcgaaggugugcugugaggcaugcuggagguaucagaagugcgaaugcugacauaaguaacgauaaagcgggugaaaagcccgcucgccggaagaccaaggguuccuguccaacguuaaucggggcagggugagucgaccccuaaggcgaggccgaaaggcguagucgaugggaaacagguuaauauuccuguacuugguguuacugcgaaggggggacggagaaggcuauguuggccgggcgacgguugucccgguuuaagcguguaggcugguuuuccaggcaaauccggaaaaucaaggcugaggcgugaugacgaggcacuacggugcugaagcaacaaaugcccugcuuccaggaaaagccucuaagcaucagguaacaucaaaucguaccccaaaccgacacagguggucagguagagaauaccaaggcgcuugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaaggacacauaaugggagggcgcuuaccacuuugugauucaugacuggggugaagucguaacaagguaaccguaggggaaccugcgguuggaucacuguggua

SEQ ID NO: 17 - DNA, 95 bp, Artificial Sequence
  Synthetic
gtaatacgactcactataggcggggtggagcagcctggtagctcgtcgggctcataacccgaagatcgtcggttcaaatccggcccccgcaacca

SEQ ID NO: 18 - DNA, 63 bp, Artificial Sequence
  Synthetic
gtaatacgactcactataggatcgaaagatttccgcggccccgaaaggggattagcgttaggt

SEQ ID NO: 19 - DNA, 64 bp, Artificial Sequence
  Synthetic
gtaatacgactcactataggatcgaaagatttccgcatccccgaaagggtacatggcgttaggt

SEQ ID NO: 20 - DNA, 65 bp, Artificial Sequence
  Synthetic
gtaatacgactcactataggatcgaaagatttccgcacccccgaaaggggtaagtggcgttaggt

SEQ ID NO: 21 - DNA, 50 bp, Artificial Sequence
  Synthetic
  misc_feature (21)..(25): n= a, t, c, or g
aagaagtaggtagcttaaccnnnnnttgagctaaccggtactaatgaacc

SEQ ID NO: 22 - DNA, 51 bp, Artificial Sequence
  Synthetic
  misc_feature (26)..(26): n is a, c, g, or t
  misc_feature (27)..(32): n=a, t, c, or g
aatcacaaagtggtaagcgccctccnnnnncttacacacccggcctatcaa

SEQ ID NO: 23 - DNA, 51 bp, Artificial Sequence
  Synthetic
  misc_feature (21)..(26): n=a, t, c, or g
aagaagtaggtagcttaaccnnnnnnttgagctaaccggtactaatgaacc

SEQ ID NO: 24 - DNA, 51 bp, Artificial Sequence; identical to SEQ ID NO: 23
  Synthetic
  misc_feature (21)..(26): n=a, t, c, or g

SEQ ID NO: 25 - DNA, 52 bp, Artificial Sequence
  Synthetic
  misc_feature (21)..(28): n= a, t, c, or g
aagaagtaggtagcttaaccnnnnnnnttgagctaaccggtactaatgaacc

SEQ ID NO: 26 - DNA, 53 bp, Artificial Sequence
  Synthetic
  misc_feature (26)..(32): n= a, t, c, or g
aatcacaaagtggtaagcgccctccnnnnnnncttacacacccggcctatcaa

SEQ ID NO: 27 - DNA, 53 bp, Artificial Sequence
  Synthetic
  misc_feature (21)..(28): n= a, t, c, or g
aagaagtaggtagcttaaccnnnnnnnnttgagctaaccggtactaatgaacc

SEQ ID NO: 28 - DNA, 54 bp, Artificial Sequence
  Synthetic
  misc_feature (26)..(33): n= a, t, c, or g
aatcacaaagtggtaagcgccctccnnnnnnnncttacacacccggcctatcaa

SEQ ID NO: 29 - DNA, 54 bp, Artificial Sequence
  Synthetic
  misc_feature (21)..(29): n= a, t, c, or g
aagaagtaggtagcttaaccnnnnnnnnnttgagctaaccggtactaatgaacc

SEQ ID NO: 30 - DNA, 55 bp, Artificial Sequence
  Synthetic
  misc_feature (26)..(34): n= a, t, c, or g
aatcacaaagtggtaagcgccctccnnnnnnnnncttacacacccggcctatcaa

SEQ ID NO: 31 - DNA, 55 bp, Artificial Sequence
  Synthetic
  misc_feature (21)..(30): n= a, t, c, or g
aagaagtaggtagcttaaccnnnnnnnnnnttgagctaaccggtactaatgaacc

SEQ ID NO: 32 - DNA, 56 bp, Artificial Sequence
  Synthetic
  misc_feature (26)..(35): n= a, t, c, or g
aatcacaaagtggtaagcgccctccnnnnnnnnnncttacacacccggcctatcaa

SEQ ID NO: 33 - DNA, 56 bp, Artificial Sequence
  Synthetic
  misc_feature (21)..(31): n= a, t, c, or g
aagaagtaggtagcttaaccnnnnnnnnnnnttgagctaaccggtactaatgaacc

SEQ ID NO: 34 - DNA, 57 bp, Artificial Sequence
  Synthetic
  misc_feature (25)..(36): n= a, t, c, or g
aatcacaaagtggtaagcgccctccnnnnnnnnnnncttacacacccggcctatcaa

SEQ ID NO: 35 - DNA, 57 bp, Artificial Sequence
  Synthetic
  misc_feature (21)..(32): n= a, t, c, or g
aagaagtaggtagcttaaccnnnnnnnnnnnnttgagctaaccggtactaatgaacc

SEQ ID NO: 36 - DNA, 58 bp, Artificial Sequence
  Synthetic
  misc_feature (26)..(37): n is a, c, g, or t
  misc_feature (26)..(37): n= a, t, c, or g
aatcacaaagtggtaagcgccctccnnnnnnnnnnnncttacacacccggcctatcaa

SEQ ID NO: 37 - DNA, 58 bp, Artificial Sequence
  Synthetic
  misc_feature (21)..(33): n= a, t, c, or g
aagaagtaggtagcttaaccnnnnnnnnnnnnnttgagctaaccggtactaatgaacc

SEQ ID NO: 38 - DNA, 59 bp, Artificial Sequence
  Synthetic
  misc_feature (26)..(38): n= a, t, c, or g
aatcacaaagtggtaagcgccctccnnnnnnnnnnnnncttacacacccggcctatcaa

SEQ ID NO: 39 - DNA, 59 bp, Artificial Sequence
  Synthetic
  misc_feature (21)..(34): n= a, t, c, or g
aagaagtaggtagcttaaccnnnnnnnnnnnnnnttgagctaaccggtactaatgaacc

SEQ ID NO: 40 - DNA, 59 bp, Artificial Sequence; identical to SEQ ID NO: 38
  Synthetic
  misc_feature (26)..(38): n= a, t, c, or g

SEQ ID NO: 41 - DNA, 60 bp, Artificial Sequence
  Synthetic
  misc_feature (21)..(35): n= a, t, c, or g
aagaagtaggtagcttaaccnnnnnnnnnnnnnnnttgagctaaccggtactaatgaacc

SEQ ID NO: 42 - DNA, 59 bp, Artificial Sequence; identical to SEQ ID NO: 38
  Synthetic
  misc_feature (26)..(38): n= a, t, c, or g

SEQ ID NO: 43 - DNA, 60 bp, Artificial Sequence
  Synthetic
gaagtaggtagcttaacccaatgaacaattggagcgttgagctaaccggtactaatgaac

SEQ ID NO: 44 - DNA, 59 bp, Artificial Sequence
  Synthetic
aatcacaaagtggtaagcgccctccactagttatcgcgcttacacacccggcctatcaa

SEQ ID NO: 45 - DNA, 60 bp, Artificial Sequence
  Synthetic
gaagtaggtagcttaacccaatgaacaattggacgttgagctaaccggtactaatgaacc

SEQ ID NO: 46 - DNA, 58 bp, Artificial Sequence
  Synthetic
aatcacaaagtggtaagcgccctccactagttatccgcttacacacccggcctatcaa

SEQ ID NO: 47 - DNA, 59 bp, Artificial Sequence
  Synthetic
gaagtaggtagcttaacccaatgaacaattggagttgagctaaccggtactaatgaacc

SEQ ID NO: 48 - DNA, 57 bp, Artificial Sequence
  Synthetic
aatcacaaagtggtaagcgccctccactagttatcgcttacacacccggcctatcaa

SEQ ID NO: 49 - DNA, 60 bp, Artificial Sequence
  Synthetic
aagaagtaggtagcttaacccaatgaacaattggattgagctaaccggtactaatgaacc

SEQ ID NO: 50 - DNA, 55 bp, Artificial Sequence
  Synthetic
aatcacaaagtggtaagcgccctccactagttatccttacacacccggcctatca

SEQ ID NO: 51 - DNA, 59 bp, Artificial Sequence
  Synthetic
aagaagtaggtagcttaacccaatgaacaattggatgagctaaccggtactaatgaacc

SEQ ID NO: 52 - DNA, 55 bp, Artificial Sequence
  Synthetic
aatcacaaagtggtaagcgccctccactagttatcttacacacccggcctatcaa

SEQ ID NO: 53 - DNA, 53 bp, Artificial Sequence
  Synthetic
  misc_feature (26)..(30): n= a, t, c, or g
aatcacaaagtggtaagcgccctccnnnnngccttacacacccggcctatcaa

SEQ ID NO: 54 - DNA, 54 bp, Artificial Sequence
  Synthetic
  misc_feature (26)..(30): n= a, t, c, or g
aatcacaaagtggtaagcgccctccnnnnngtccttacacacccggcctatcaa

SEQ ID NO: 55 - DNA, 53 bp, Artificial Sequence
  Synthetic
  misc_feature (21)..(26): n= a, t, c, or g
aagaagtaggtagcttaaccnnnnnngcttgagctaaccggtactaatgaacc

SEQ ID NO: 56 - DNA, 54 bp, Artificial Sequence
  Synthetic
  misc_feature (26)..(31): n= a, t, c, or g
aatcacaaagtggtaagcgccctccnnnnnngccttacacacccggcctatcaa

SEQ ID NO: 57 - DNA, 54 bp, Artificial Sequence
  Synthetic
  misc_feature (21)..(27): n= a, t, c, or g
aagaagtaggtagcttaaccnnnnnnngcttgagctaaccggtactaatgaacc

SEQ ID NO: 58 - DNA, 55 bp, Artificial Sequence
  Synthetic
  misc_feature (26)..(32): n= a, t, c, or g
aatcacaaagtggtaagcgccctccnnnnnnngccttacacacccggcctatcaa

SEQ ID NO: 59 - DNA, 55 bp, Artificial Sequence
  Synthetic
  misc_feature (21)..(28): n= a, t, c, or g
aagaagtaggtagcttaaccnnnnnnnngcttgagctaaccggtactaatgaacc

SEQ ID NO: 60 - DNA, 56 bp, Artificial Sequence
  Synthetic
  misc_feature (26)..(33): n= a, t, c, or g
aatcacaaagtggtaagcgccctccnnnnnnnngccttacacacccggcctatcaa

SEQ ID NO: 61 - DNA, 56 bp, Artificial Sequence
  Synthetic
  misc_feature (21)..(29): n= a, t, c, or g
aagaagtaggtagcttaaccnnnnnnnnngcttgagctaaccggtactaatgaacc

SEQ ID NO: 62 - DNA, 57 bp, Artificial Sequence
  Synthetic
  misc_feature (26)..(34): n= a, t, c, or g
aatcacaaagtggtaagcgccctccnnnnnnnnngccttacacacccggcctatcaa

SEQ ID NO: 63 - DNA, 25 bp, Artificial Sequence
  Synthetic
ggagggcgcttaccactttgtgatt

SEQ ID NO: 64 - DNA, 25 bp, Artificial Sequence
  Synthetic
ggttaagctacctacttcttttgca

SEQ ID NO: 65 - DNA, 29 bp, Artificial Sequence
  Synthetic
ggaacgttgaagacgacgacgttgatagg

SEQ ID NO: 66 - DNA, 52 bp, Artificial Sequence
  Synthetic
cctatcaacgtcgtcgtcttcaacgttccacggttcattagtaccggttagc

SEQ ID NO: 67 - DNA, 29 bp, Artificial Sequence
  Synthetic
ggaacgttgaagacgacgacgttgatagg

SEQ ID NO: 68 - DNA, 24 bp, Artificial Sequence
  Synthetic
cacggttcattagtaccggttagc

SEQ ID NO: 69 - DNA, 39 bp, Artificial Sequence
  Synthetic
agatggatccggaacgttgaagacgacgacgttgatagg

SEQ ID NO: 70 - DNA, 34 bp, Artificial Sequence
  Synthetic
ggatccatctcacggttcattagtaccggttagc

SEQ ID NO: 71 - DNA, 40 bp, Artificial Sequence
  Synthetic
gctcttcagcgggaacgttgaagacgacgacgttgatagg

SEQ ID NO: 72 - DNA, 36 bp, Artificial Sequence
  Synthetic
ggctcttcacgccacggttcattagtaccggttagc

SEQ ID NO: 73 - DNA, 26 bp, Artificial Sequence
  Synthetic
agtgggttgcaaaagaagtaggtagc

SEQ ID NO: 74 - DNA, 27 bp, Artificial Sequence
  Synthetic
ccagtcatgaatcacaaagtggtaagc

SEQ ID NO: 75 - RNA, 15 nt, Artificial Sequence
  Synthetic
caaugaacaauugga

SEQ ID NO: 76 - RNA, 21 nt, Artificial Sequence
  Synthetic
uuucaucaccacagaucuaug

SEQ ID NO: 77 - RNA, 49 nt, Artificial Sequence
  Synthetic
aagaaguagguagcuuaaccuucgaaaaaaaacgaugcguugagcuaac

SEQ ID NO: 78 - RNA, 41 nt, Artificial Sequence
  Synthetic
gguguguaagcgcagaaaaaaaaaggagggcgcuuaccacu

SEQ ID NO: 79 - RNA, 55 nt, Artificial Sequence
  Synthetic
aagaaguagguagcuuaaccuucgaaaaaaaacgaugcguugagcuaaccgguac

SEQ ID NO: 80 - RNA, 40 nt, Artificial Sequence
  Synthetic
ggccgggggaagcgcagaaaaaaaaaggagggcgcaccac

SEQ ID NO: 81 - RNA, 55 nt, Artificial Sequence
  Synthetic
aagaaguagguagcuuaacccaaugaacaauuggaugcguugagcuaaccgguac

SEQ ID NO: 82 - RNA, 46 nt, Artificial Sequence
  Synthetic
ggccggguguguaagcgcagauaacuaguggagggcgcuuaccacu

SEQ ID NO: 83 - RNA, 11 nt, Artificial Sequence
  Synthetic
ugcguugagcu

SEQ ID NO: 84 - RNA, 12 nt, Artificial Sequence
  Synthetic
uguguaagcgca

SEQ ID NO: 85 - RNA, 15 nt, Artificial Sequence
  Synthetic
caaugaacaauugga

SEQ ID NO: 86 - RNA, 10 nt, Artificial Sequence
  Synthetic
gauaacuagu

SEQ ID NO: 87 - RNA, 10 nt, Artificial Sequence
  Synthetic
aggaaagagc

SEQ ID NO: 88 - RNA, 12 nt, Artificial Sequence
  Synthetic
ccuuuggauccg

SEQ ID NO: 89 - RNA, 10 nt, Artificial Sequence
  Synthetic
gucuugagcu

SEQ ID NO: 90 - RNA, 11 nt, Artificial Sequence
  Synthetic
uguguaaggac

SEQ ID NO: 91 - RNA, 10 nt, Artificial Sequence
  Synthetic
aggaaagagc

SEQ ID NO: 92 - RNA, 10 nt, Artificial Sequence
  Synthetic
ugguacagua

SEQ ID NO: 93 - RNA, 10 nt, Artificial Sequence
  Synthetic
ugguaccgua

SEQ ID NO: 94 - RNA, 10 nt, Artificial Sequence
  Synthetic
ugguccagua

SEQ ID NO: 95 - RNA, 11 nt, Artificial Sequence
  Synthetic
caggguacacc

SEQ ID NO: 96 - RNA, 11 nt, Artificial Sequence
  Synthetic
agguaagacuu

SEQ ID NO: 97 - RNA, 11 nt, Artificial Sequence
  Synthetic
aacauugauau

SEQ ID NO: 98 - RNA, 11 nt, Artificial Sequence
  Synthetic
ugguuuucuug

SEQ ID NO: 99 - RNA, 11 nt, Artificial Sequence
  Synthetic
auagccauaac

SEQ ID NO: 100 - RNA, 12 nt, Artificial Sequence
  Synthetic
uaaagaggaaga

SEQ ID NO: 101 - RNA, 13 nt, Artificial Sequence
  Synthetic
gccacgagcccuc

SEQ ID NO: 102 - RNA, 13 nt, Artificial Sequence
  Synthetic
gucuaguagucca

SEQ ID NO: 103 - RNA, 14 nt, Artificial Sequence
  Synthetic
ggcauguaggggcc

SEQ ID NO: 104 - RNA, 14 nt, Artificial Sequence
  Synthetic
uugucgcucguuua

SEQ ID NO: 105 - RNA, 15 nt, Artificial Sequence
  Synthetic
uccuagagaguguau

SEQ ID NO: 106 - RNA, 15 nt, Artificial Sequence
  Synthetic
gccgaagccggagcg

SEQ ID NO: 107 - RNA, 15 nt, Artificial Sequence
  Synthetic
cgcugugggucugug

SEQ ID NO: 108 - RNA, 22 nt, Artificial Sequence
  Synthetic
uaaccaugacaugguuugagcu

SEQ ID NO: 109 - RNA, 24 nt, Artificial Sequence
  Synthetic
uguguaagccggcuucggaaggag

SEQ ID NO: 110 - RNA, 22 nt, Artificial Sequence
  Synthetic
uaacccgagaaaggauugagcu

SEQ ID NO: 111 - RNA, 24 nt, Artificial Sequence
  Synthetic
uguguaagccggcuucggaaggag

SEQ ID NO: 112 - RNA, 10 nt, Artificial Sequence
  Synthetic
ugguacagua

SEQ ID NO: 113 - RNA, 12 nt, Artificial Sequence
  Synthetic
ccuuuggauccg